SEQUENCE LISTING (157 sequences)

SEQ ID NO: 1 - protein, 251 aa, artificial
  phage display generated antibody
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGRGTMVTVSSGGGGSGGGGSGGGGSSELTQDPAVSVALGQTVRITCQGDSLRSYYASWYQQKPGQAPVLVIYGKNKRPSGIPDRFSGSSSGNTASLTITGAQAEDEADYYCHSRDSSGNHVLFGGGTKLTVLG

SEQ ID NO: 2 - protein, 251 aa, artificial
  phage display generated antibody
GVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSSGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLG

SEQ ID NO: 3 - protein, 251 aa, artificial
  phage display generated antibody
EVQLVQSGAEVKKPGESLTISCKGSGYNFFNYWIGWVRQMPGKDLEWMGIIYPTDSDTRYSPSFQGQVTISVDKSISTAYLQWSSLKASDTAMYYCARSIRYCPGGRCYSGYYGMDVWGQGTMVTVSSGGGSSGGGGSGGGGSSELTQDPAVSVALGQTVRITCRGDSLRNYYASWYQQKPGQAPVLVIYGKNNRPSGIPDRFSGSSSGNTASLTITGAQAEDEADYYCNSRDSSGNHMVFGGGTKLTVLG

SEQ ID NO: 4 - protein, 251 aa, artificial
  phage display generated antibody
GVQLVESGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGRGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 5 - protein, 251 aa, artificial
  phage display generated antibody
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTLVTVSSGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLG

SEQ ID NO: 6 - protein, 251 aa, artificial
  phage display generated antibody
EVQLVQSGAEVKKPGESLTISCKGPGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 7 - protein, 245 aa, artificial
  phage display generated antibody
QVQLVQSGAEVRKPGASVKVSCKTSGYTFRNYDINWVRQAPGQGLEWMGRISGHYGNTDHAQKFQGRFTMTKDTSTSTAYMELRSLTFDDTAVYYCARSQWNVDYWGRGTLVTVSSGGGGSGGGGSGGGGSALNFMLTQPHSVSESPGKTVTISCTRSSGSIASNYVQWYQQRPGSSPTTVIFEDNRRPSGVPDRFSGSIDTSSNSASLTISGLKTEDEADYYCQSFDSTNLVVFGGGTKVTVLG

SEQ ID NO: 8 - protein, 249 aa, artificial
  phage display generated antibody
EVQLVESGGGVVQPGRSLRLSCAASGFTFSDFAMHWVRQIPGKGLEWLSGLRHDGSTAYYAGSVKGRFTISRDNSRNTVYLQMNSLRAEDTATYYCVTGSGSSGPHAFPVWGKGTLVTVSSGGGGSGGGGSGGGGSALSYVLTQPPSASGTPGQRVTISCSGSNSNIGTYTVNWFQQLPGTAPKLLIYSNNQRPSGVPDRFSGSKSGTSASLAISGLQSEDEADYYCAAWDDSLNGPVFGGGTKVTVLG

SEQ ID NO: 9 - protein, 253 aa, artificial
  phage display generated antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGMGYYGSGGYYPDDAFDVWGQGTMVTVSSGGGGSGGGGSGGGGSALSSELTQDPDVSMALGQTVTISCRGDSLKRFYASWYHQKPGQAPVLVFYGKENRPSGIPDRFSGSDSGDTASLTITGAQAEDEGDYYCHTQDTSARQYVFGSGTKVTVLG

SEQ ID NO: 10 - protein, 251 aa, artificial
  phage display generated antibody
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGRGTLVTVSSGGGGSGGGGSGGGGSALSSELTQDPAVSVALGQTVRITCQGDSLRNYYASWYQQKPGQAPLLVMFGKNNRPSEIPGRFSGSSSGNTASLTITGAQAEDEADYYCNSRDRNSHQWVFGGGTKLTVLG

SEQ ID NO: 11 - protein, 245 aa, artificial
  phage display generated antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASSPYSSRWYSFDPWGQGTMVTVSSGGGGSGGGGSGGGGSALSYELTQPPSVSVSPGQTATITCSGDDLGNKYVSWYQQKPGQSPVLVIYQDTKRPSGIPERFSGSNSGNIATLTISGTQAVDEADYYCQVWDTGTVVFGGGTKLTVLG

SEQ ID NO: 12 - protein, 252 aa, artificial
  phage display generated antibody
QVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGKGTLVTVSSGGGGSGGGGSGGGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNHHWVFGGGTKVTVLG

SEQ ID NO: 13 - protein, 253 aa, artificial
  phage display generated antibody
EVQLVQSGAEVKKPGASVKVSCKASGYSFTNYGLDWVRQAPGQGLEWMGWISPYTGYTNYAQKFQGRVTMTTDKSTSTAYMDLRSLRSDDTAVYYCAREIFSHCTGGSCYPFDSWGRGTMVTVSSGGGGSGGGGSGGGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNHRNWVFGGGTKVTVLG

SEQ ID NO: 14 - protein, 247 aa, artificial
  phage display generated antibody
QVQLVESGGGLVKPGGSLRLSCAASGFTFSSHTMNWVRQAQGKGLEWVSSISGSGRYIYYSDSVKGRFTISRDAAKNSLYLQMNNLRAEDTAVYYCTRAKFGDYLFDSWGQGTLVTVSSGGGGSGGGGSGGGGSALNFMLTQPHSVSQSPGKTVTISCTRSSGRIASNFVQWYQQRPGSAPTTVIYEDNRRPSGVPDRFSGSIDSSSNSASLTISGLKTEDEADYYCQSYDARYQVFGTGTKVTVLG

SEQ ID NO: 15 - protein, 251 aa, artificial
  phage display generated antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPVPPWADWYYFDYWGRGTMVTVSSGGGGSGGGGSGGGGSAQAVLTQPSSVSGAPGQRVTISCTGSRSNFGAGYDVHWYQQFPGTAPKLLIYGNTNRPSGVPDRFSGSRSGTSASLAITGLQAEDEADYYCQSYDSNLSGSVFGGGTKVTVLG

SEQ ID NO: 16 - protein, 252 aa, artificial
  phage display generated antibody
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGKGTLVTVSSGGGGSGGGGSGGGGSALSSELTQDPAVSVALGQTVRITCQGDSLRNYYASWYQQKPGQAPVLVLYSKNSRPSGVPDRFSGSSSGTTASLTISGAQAEDEADYYCNSRDTSGDLRWVFGGGTKLTVLG

SEQ ID NO: 17 - protein, 251 aa, artificial
  phage display generated antibody
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGQGTLVTVSSGGGGSGGGGSGGGGSALSSELTQDPAVSVALGQTVRITCQGDSLRNYYASWYQQKPGQAPLLVMFGKNNRPSEIPGRFSGSSSGNTASLTITGAQAEDEADYYCNSRDSNSHQWVFGGGTKLTVLG

SEQ ID NO: 18 - protein, 253 aa, artificial
  phage display generated antibody
QVQLVQSGAEVKKPGASVKVSCKASGYSFTNYGLNWVRQAPGQGLEWMGWISPYTGYTNYAQKFQGRVTMTSDKSTSTAYMDLRSLRSDDTAIYYCAREIFSHCSGGSCYPFDYWGQGTLVTVSSGGGGSGGGGSGGGGSALSSELTQDPAVSVALGQTVRITCQGDSLRSYYASWYQQKPGQAPLLVIYGRNNRPSGIPDRFSGSSSGNTASLTITGAQAEDEADYYCNSRDSSTNHGNWVFGGGTQLTVLS

SEQ ID NO: 19 - protein, 252 aa, artificial
  phage display generated antibody
QVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGRGTMVTVSSGGGGSGGGGSGGGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNLNWVFGGGTQLTVLS

SEQ ID NO: 20 - DNA, 753 bp, artificial
  phage display generated antibody
gaagtgcagctggtgcagtctggagcagaggtgaaaaagcccggggagtctctgacaatctcctgtaagggttctgggtacaactttttcaactactggatcggctgggtgcgccagatgcccgggaaaggcctggagtggatggggatcatctatcctactgactctgataccagatatagcccgtccttccaaggccaggtcaccatttcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccgggggacaatggtcaccgtctcttcaggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaataagcggccctcagggatcccagaccgattctctggctccagctcaggaaacacagcttccttgaccatcactggggctcaggcggaagatgaggctgactattactgtcattcccgggacagcagtggtaaccatgtgcttttcggcggagggaccaagctgaccgtcctaggt

SEQ ID NO: 21 - DNA, 753 bp, artificial
  phage display generated antibody
ggggtgcagctggtgcagtctggggcagaggtgaaaaagcccggggagtctctgacaatctcctgtaagggttctggatacaactttttcaactactggatcggctgggtgcgccagatgcccgggaaaggcctggagtggatggggatcatctatcctactgactctgataccagatatagcccgtccttccaaggctaggtcaccatctcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccaggggacaatggtcaccgtctcgagtggtggaggcggttcaggcggaggtggcagcggcggtggcggatcgtctgagttgactcaggaccctgctgtgtctgtggccttgggacagacagtcaggatcacttgccaaggagacagtctcagaagctattacacaaactggttccagcagaagccaggacaggcccctctacttgtcgtctatgctaaaaataagcggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggt

SEQ ID NO: 22 - DNA, 753 bp, artificial
  phage display generated antibody
gaagtgcagctggtgcagtctggggcagaggtgaaaaagcccggggagtctctgacaatctcctgcaagggttctggatacaactttttcaactactggatcggctgggtgcgccagatgcccgggaaagacctggagtggatggggatcatctatcctactgactctgataccagatatagcccgtccttccaaggccaggtcacgatttcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccaggggacaatggtcaccgtctcgagtggtggaggcagttcaggcggaggtggcagcggcggtggcggatcgtctgagctgactcaggaccctgctgtgtctgtggccttgggacagacagtcaggatcacatgccgaggagacagcctcagaaactattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatatggtattcggcggagggaccaagctgaccgtcctaggt

SEQ ID NO: 23 - DNA, 753 bp, artificial
  phage display generated antibody
ggggtgcagctggtggagtctggggcagaggtgaaaaagcccggggagtctctgacaatctcctgtaagggttctggatacaactttttcaactactggatcggctgggtgcgccagatgcccgggaaaggcctggagtggatggggatcatctatcctactgactctgataccagatatagcccgtccttccaaggccaggtcaccatctcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccggggaaccctggtcaccgtctcctcaggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggt

SEQ ID NO: 24 - DNA, 753 bp, artificial
  phage display generated antibody
gaagtgcagctggtgcagtctggagcagaggtgaaaaagcccggggagtctctgacaatctcctgtaagggttctggatacaactttttcaactactggatcggctgggtgcgccagatgcccgggaaaggcctggagtggatggggatcatctatcctactgactctgataccagatatagcccgtccttccaaggccaggtcaccatttcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccagggcaccctggtcaccgtctcctcaggtggaggcggttcaggcggaggtggcagcggcggtggcggatcgtctgagctgactcaggaccctgctgtgtctgtggccttgggacagacagtcaggatcacttgccaaggagacagtctcagaagctattacacaaactggttccagcagaagccaggacaggcccctctacttgtcgtctatgctaaaaataagcggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggt

SEQ ID NO: 25 - DNA, 753 bp, artificial
  phage display generated antibody
gaggtgcagctggtgcagtctggggcagaggtgaaaaagcccggggagtctctgacaatctcctgtaagggtcctggatacaactttttcaactactggatcggctgggtgcgccagatgcccgggaaaggcctggagtggatggggatcatctatcctactgactctgataccagatatagcccgtccttccaaggccaggtcaccatctcagtcgacaagtccattagcaccgcctatctgcagtggagcagcctgaaggcctccgacaccgccatgtattactgtgcgagatccattagatactgtcctggtggtaggtgctactccggttactacggtatggacgtctggggccaaggaaccatggtcaccgtctcctcaggtggaggcggttcaggcggaggtggcagcggcggtggcggatcgtctgagctgactcaggaccctgctgtgtctgtggccttgggacagacggtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggt

SEQ ID NO: 26 - DNA, 735 bp, artificial
  phage display generated antibody
caggtccagctggtgcagtctggggctgaagtgaggaagcctggggcctcagtgaaggtctcctgcaagacttcaggttacacctttaggaactatgatatcaactgggtgcgacaggcccctggacaagggcttgagtggatgggaaggatcagtggtcactatggcaacacagaccatgcacagaaattccagggcagattcaccatgaccaaagacacatccacgagcacagcctacatggaactgaggagcctgacatttgacgacacggccgtatattactgtgcgagaagtcagtggaacgttgactactggggccgaggaaccctggtcaccgtctcgagtggaggcggcggttcaggcggaggtggctctggcggtggcggaagtgcacttaattttatgctgactcagccccactctgtgtcggagtctccggggaagacggtgaccatctcctgcacccgcagcagtggcagcattgctagcaattatgtgcagtggtaccagcagcgcccgggcagttcccccaccactgtgatctttgaagataaccgaagaccctctggggtccctgatcggttttctggctccatcgacacctcctccaactctgcctccctcaccatctctggactgaagactgaggacgaggctgactactactgtcagtcttttgatagcaccaatcttgtggtgttcggcggagggaccaaggtcaccgtcctaggt

SEQ ID NO: 27 - DNA, 774 bp, artificial
  phage display generated antibody
gaggtgcagctggtggagtctgggggaggcgtggtccagcctgggaggtccctgagactctcctgtgcagcgtctggcttcactttcagtgattttgccatgcactgggtccgccagattccaggcaaggggctggagtggctgtcaggattacggcatgatggaagtacggcttactatgcagggtccgtgaagggccgcttcaccatctccagagacaattccaggaatactgtatatctccaaatgaatagcctgagggccgaggacacggctacgtattactgtgtgacagggagcggtagctccggtccccacgcttttcctgtctggggcaaaggcaccctggtcaccgtctcgagtggaggcggcggttcaggcggaggtggctctggcggtggcggaagtgcactttcctatgtgctgactcagccaccctcagcgtctgggacccccgggcagagggtcaccatctcttgttctggaagcaactccaacatcgggacttatactgtaaattggttccagcagctcccaggaacggcccccaaactcctcatctacagtaataatcagcggccctcaggggtccctgaccgattctctggctccaagtctggcacctcagcctccctggccatcagtgggctccagtctgaggatgaggctgattattactgtgcagcaatgggatgacagcctgaatggtccggttttcggcggagggaccaaggtcaccgtcctaggtgcggccgcacatcatcatcaccatca

SEQ ID NO: 28 - DNA, 759 bp, artificial
  phage display generated antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaaggaatgggatactatggttcgggaggttattatccggatgatgcttttgatgtctggggccaggggacaatggtcaccgtctcgagtggaggcggcggttcaggcggaggtggctctggcggtggcggaagtgcactttcttctgagctgactcaggaccctgatgtgtctatggccttgggtcagacagtcaccatttcatgccgaggagacagcctcaaaagattttatgcaagttggtatcaccagaagccaggacaggcccctgtccttgtcttctatggtaaagaaaatcggccctcagggatcccagaccggttctctggctccgactctggagacacagcctccttgaccatcactggggctcaggcggaagatgagggtgactattactgtcacactcaggacaccagtgctcgccaatatgtcttcgggagtgggaccaaggtcaccgtcctaggt

SEQ ID NO: 29 - DNA, 753 bp, artificial
  phage display generated antibody
gaggtgcagctggtgcagtcgggggctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggccgaggcaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaaactactatgcaagttggtaccagcagaagccagggcaggcccctctccttgtcatgtttggtaagaacaaccggccctcagagatcccaggccgattctctggctccagttcgggaaacacagcttccttgaccatcactggggctcaggcggaagatgaggctgactattactgtaattctcgagacagaaacagtcatcaatgggtgttcggcggagggaccaagctgaccgtcctaggt

SEQ ID NO: 30 - DNA, 735 bp, artificial
  phage display generated antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagtagtccctatagcagcaggtggtactcgttcgacccctggggccaagggacaatggtcaccgtctcgagtggaggcggcggttcaggcggaggtggctctggcggtggcggaagtgcactttcctatgagctgactcagccaccctcagtgtccgtgtccccaggacagacagccaccatcacctgctctggagatgacttggggaataaatatgtttcgtggtatcaacagaagccaggccagtcccctgtgctggtcatctatcaagataccaagcggccctcagggatccctgagcgattctctggctccaactctgggaacatagccactctgaccatcagcgggacccaggctgtggatgaggctgactattattgtcaggtgtgggacaccggcactgtggttttcggcggcgggaccaagctgaccgtcctaggt

SEQ ID NO: 31 - DNA, 756 bp, artificial
  phage display generated antibody
caggtccagctggtgcagtctggggctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggcaaaggaaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatcattgggtgttcggcggagggaccaaggtcaccgtcctaggt

SEQ ID NO: 32 - DNA, 759 bp, artificial
  phage display generated antibody
gaggtccagctggtgcagtctggggctgaggtgaagaagcctggggcctcagtgaaggtctcctgtaaggcctctggttactcttttaccaactatggtctcgactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggcagagggacaatggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatcggaattgggtgttcggcggagggaccaaggtcaccgtcctaggt

SEQ ID NO: 33 - DNA, 741 bp, artificial
  phage display generated antibody
caggtgcagctggtggagtctgggggaggcctggtcaagcctggggggtccctgagactctcctgtgcagcctctggattcaccttcagcagccacaccatgaactgggtccgccaggctcaagggaaggggctggagtgggtctcatccattagtggtagtggtcgttacatttactattcagactcagtgaagggccggttcaccatctccagagacgccgccaagaactctctgtatctgcaaatgaacaacctgagagccgaggacacggctgtctattactgtacgagagcgaaattcggtgactacctctttgactcctggggccagggcaccctggtcaccgtctcgagtggaggcggcggttcaggcggaggtggctctggcggtggcggaagtgcacttaattttatgctgactcagccccactctgtgtcgcagtctccggggaagacggtaaccatctcctgcacccgcagtagtggcagaattgccagcaactttgtgcagtggtaccagcagcgcccgggcagtgcccccaccactgtgatctatgaggataaccgacgaccctctggggtccctgatcggttctctggctccatcgacagctcctccaactctgcctccctcaccatctctggactaaagactgaggacgaggctgactactattgtcagtcttatgatgccagatatcaagtcttcggaactgggaccaaggtcaccgtcctaggg

SEQ ID NO: 34 - DNA, 753 bp, artificial
  phage display generated antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagcggtggtagcacatactacgcagactccgtgaagggccggttcaccatctccagagacaattccaagaacacgctgtatctgcaaatgaacagcctgagagccgaggacacggccgtgtattactgtgcgaggtcgcctgtcccgccgtgggcggactggtactactttgattattggggccgggggacaatggtcaccgtctcgagtggaggcggcggttcaggcggaggtggctctggcggtggcggaagtgcacaggctgtgctgactcagccgtcctcagtgtctggggccccagggcagagggtcaccatctcctgcactgggagcaggtccaacttcggggcaggttatgatgtacactggtaccagcagtttccaggaacagcccccaaactcctcatctatggtaacaccaatcggccctcaggggtccctgaccgattctctggctccaggtctggcacctcagcctccctggccatcactgggctccaggctgaggatgaggctgattattactgccagtcatatgacagcaacctgagtggttcggtgttcggcggcgggaccaaggtcaccgtcctaggt

SEQ ID NO: 35 - DNA, 756 bp, artificial
  phage display generated antibody
gaggtccagctggtacagtctggagctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggcaaaggaaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaaactattatgcaagctggtaccagcagaagccagggcaggcccctgtccttgtcctctacagtaaaaacagccggccctctggggtcccagaccgattctctggctccagctcaggaaccacagcttccttgacaatcagtggggctcaggcggaagatgaggctgactattactgtaattctcgggacaccagtggtgaccttcgctgggtgttcggcggagggaccaagctgaccgtcctaggt

SEQ ID NO: 36 - DNA, 753 bp, artificial
  phage display generated antibody
gaggtccagctggtgcagtctggggctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggccagggcaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaaactactatgcaagttggtaccagcagaagccagggcaggcccctctccttgtcatgtttggtaagaacaaccggccctcagagatcccaggccgattctctggctccagttcgggaaacacagcttccttgaccatcactggggctcaggcggaagatgaggctgactattactgtaattctcgagacagtaacagtcatcaatgggtgttcggcggagggaccaagctgaccgtcctaggt

SEQ ID NO: 37 - DNA, 759 bp, artificial
  phage display generated antibody
caggtgcagctggtgcagtctggggctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgacttcagataaatccacgagcacagcctacatggacctgaggagtctgagatctgacgacacggccatttattattgtgcgagagagattttctcccattgtagtggtggtagttgctacccttttgactactggggccagggaaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaagctattatgcaagctggtaccagcagaagccaggacaggcccctctacttgtcatctatggtagaaacaaccggccctcagggatcccagaccgattctctggctccagctcaggaaacacagcttccttgaccatcactggggctcaggcggaagatgaggctgactattactgtaactcccgggacagcagtactaaccatgggaattgggtgttcggcggagggacccagctcaccgttttaagt

SEQ ID NO: 38 - DNA, 756 bp, artificial
  phage display generated antibody
caggtccagctggtgcagtctggggctgaggtgaagaagcctggggcctcagtgaaggtctcctgtaaggcctctggttactcttttaccaactatggtctcaactgggtgcgacaggcccctggacagggacttgagtggatgggatggatcagcccttacactggttacacaaattatgcacagaagttccagggcagagtcaccatgaccacagataaatccacgagcacagcctacatggacctgaggagtctgagatctgacgacaccgccgtttattactgtgcgagagagattttttctcattgtactggtggcagttgctacccttttgactcctggggcagggggacaatggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaacctcaattgggtgttcggcggagggacccagctcaccgttttaagt

SEQ ID NO: 39 - protein, 15 aa, artificial
  Vh-Vl linker
GGGGSGGGGSGGGGS

SEQ ID NO: 40 - DNA, 27 bp, artificial
  primer
gtccttccaaggccaggtcacgatctc

SEQ ID NO: 41 - DNA, 27 bp, artificial
  primer
gagatcgtgacctggccttggaaggac

SEQ ID NO: 42 - DNA, 23 bp, artificial
  primer
ccaagctgaccgtcctaggtgag

SEQ ID NO: 43 - DNA, 23 bp, artificial
  primer
ctcacctaggacggtcagcttgg

SEQ ID NO: 44 - DNA, 33 bp, artificial
  primer
cgtccttccaaggccaagtcaccatctcagtcg

SEQ ID NO: 45 - DNA, 33 bp, artificial
  primer
cgactgagatggtgacttggccttggaaggacg

SEQ ID NO: 46 - DNA, 41 bp, artificial
  primer
ctctccacaggcgtgcactcctcgtctgagctgactcagga

SEQ ID NO: 47 - DNA, 60 bp, artificial
  primer
ctattccttaattaagttagatctattctgactcacctaggacggtcagcttggtccctc

SEQ ID NO: 48 - DNA, 41 bp, artificial
  primer
ctctccacaggcgcgcactccggggtgcagctggtgcagtc

SEQ ID NO: 49 - DNA, 27 bp, artificial
  primer
tgaggagacggtgaccattgtcccctg

SEQ ID NO: 50 - DNA, 49 bp, artificial
  primer
ctttctctccacaggcgtgcactcctctgagctgactcaggaccctgct

SEQ ID NO: 51 - DNA, 64 bp, artificial
  primer
ctattccttaattaagttagatctattctgactcacctaggacggtcagcttggtccctccgcc

SEQ ID NO: 52 - DNA, 68 bp, artificial
  primer
ctctccacaggcgcgcactccggggtgcagctggtggagtctgaggagacggtgaccagggttccccg

SEQ ID NO: 53 - DNA, 27 bp, artificial
  primer
tgaggagacggtgaccagggttccccg

SEQ ID NO: 54 - DNA, 41 bp, artificial
  primer
ctctccacaggcgcgcactccgaagtgcagctggtgcagtc

SEQ ID NO: 55 - DNA, 27 bp, artificial
  primer
tgaggagacggtgaccagggtgccctg

SEQ ID NO: 56 - DNA, 41 bp, artificial
  primer
gatcgatcgcgcgcactccgaggtgcagctggtgcagtctg

SEQ ID NO: 57 - DNA, 31 bp, artificial
  primer
gatcgatcggtgaccatggttccttggcccc

SEQ ID NO: 58 - DNA, 39 bp, artificial
  primer
gatcgatcgtgcactcctctgagctgactcaggaccctg

SEQ ID NO: 59 - DNA, 65 bp, artificial
  primer
gatcgatcttaattaagttagatctattctgactcacctaggacggtcagcttggtccctccgcc

SEQ ID NO: 60 - DNA, 42 bp, artificial
  primer
ggatcttggcgcgcactccgaggtgcagctggtggagtctgg

SEQ ID NO: 61 - DNA, 38 bp, artificial
  primer
gatcgatcggtgaccattgtccctcggccccagatatc

SEQ ID NO: 62 - DNA, 40 bp, artificial
  primer
gatcgatcgtgcactcccagtctgtgctgactcagccacc

SEQ ID NO: 63 - DNA, 62 bp, artificial
  primer
gatcgatcttaattaagttagatctattctgactcacctaggacggtcagcttggtccctcc

SEQ ID NO: 64 - DNA, 40 bp, artificial
  primer
gatcgatcgcgcgcactcccaggtccagctggtgcagtct

SEQ ID NO: 65 - DNA, 37 bp, artificial
  primer
gatcgatcggtgacccagggttcctcggccccagtag

SEQ ID NO: 66 - DNA, 38 bp, artificial
  primer
gatcgatcgtgcactccgcacttaattttatgctgact

SEQ ID NO: 67 - DNA, 58 bp, artificial
  primer
gatcgatcttaattaagttagatctattctgactcacctaggacggtgaccttggtcc

SEQ ID NO: 68 - DNA, 40 bp, artificial
  primer
gatcgatcgcgcgcactccgaggtgcagctggtggagtct

SEQ ID NO: 69 - DNA, 38 bp, artificial
  primer
gatcgatcggtgaccagggtgcctttgccccagacagg

SEQ ID NO: 70 - DNA, 39 bp, artificial
  primer
gatcgatcgtgcactccgcactttcctatgtgctgactc

SEQ ID NO: 71 - DNA, 63 bp, artificial
  primer
gatcgatcttaattaaaagttagatctattctgactcacctaggacggtgaccttggtccctc

SEQ ID NO: 72 - DNA, 41 bp, artificial
  primer
gatcgatcgcgcgcactccgaggtgcagctgttggagtctg

SEQ ID NO: 73 - DNA, 38 bp, artificial
  primer
gatcgatcggtgaccattgtcccctggccccagacatc

SEQ ID NO: 74 - DNA, 39 bp, artificial
  primer
gatcgatcgtgcactccgcactttcttctgagctgactc

SEQ ID NO: 75 - DNA, 61 bp, artificial
  primer
gatcgatcttaattaagttagatctattctgactcacctaggacggtgaccttggtcccac

SEQ ID NO: 76 - DNA, 45 bp, artificial
  primer
gatcgatcgcgcgcactccgaggtgcagctggtgcagtcgggggc

SEQ ID NO: 77 - DNA, 34 bp, artificial
  primer
gatcgatcggtgaccagggtgcctcggccccagg

SEQ ID NO: 78 - DNA, 42 bp, artificial
  primer
gatcgatcgtgcactccgcactttcttctgagctgactcagg

SEQ ID NO: 79 - DNA, 72 bp, artificial
  primer
gatcgatcttaattaagttagatctattctgactcacctaggacggtcagcttggtccctccgccgaacacc

SEQ ID NO: 80 - DNA, 41 bp, artificial; identical to SEQ ID NO: 72
  primer

SEQ ID NO: 81 - DNA, 36 bp, artificial
  primer
gatcgatcggtgaccattgtcccttggccccagggg

SEQ ID NO: 82 - DNA, 39 bp, artificial
  primer
gatcgatcgtgcactccgcactttcctatgagctgactc

SEQ ID NO: 83 - DNA, 65 bp, artificial
  primer
gatcgatcttaattaagttagatctattctgactcacctaggacggtcagcttggtcccgccgcc

SEQ ID NO: 84 - DNA, 39 bp, artificial
  primer
gatcgatcgcgcgcactcccaggtccagctggtgcagtc

SEQ ID NO: 85 - DNA, 38 bp, artificial
  primer
gatcgatcggtgaccagggttcctttgccccaggagtc

SEQ ID NO: 86 - DNA, 39 bp, artificial; identical to SEQ ID NO: 74
  primer

SEQ ID NO: 87 - DNA, 72 bp, artificial
  primer
gatcgatcttaattaagttagatctattgtgactcacctaggacggtgaccttggtccctccgccgaacacc

SEQ ID NO: 88 - DNA, 41 bp, artificial
  primer
gatcgatcgcgcgcactccgaggtccagctggtgcagtctg

SEQ ID NO: 89 - DNA, 38 bp, artificial
  primer
gatcgatcggtgaccattgtccctctgccccaggagtc

SEQ ID NO: 90 - DNA, 41 bp, artificial
  primer
gatcgatcgtgcactccgcactttcttctgsgctgactcag

SEQ ID NO: 91 - DNA, 66 bp, artificial
  primer
gatcgatcttaattaagttagatctattctgactcacctaggacggtgaccttggtccctccgccg

SEQ ID NO: 92 - DNA, 41 bp, artificial
  primer
gatcgatcgcgcgcactccaggtgcagctggtggagtctgg

SEQ ID NO: 93 - DNA, 38 bp, artificial
  primer
gatcgatcggtgaccagggtgccctggccccaggagtc

SEQ ID NO: 94 - DNA, 39 bp, artificial
  primer
gatcgatcgtgcactccgcacttaattttatgctgactc

SEQ ID NO: 95 - DNA, 68 bp, artificial
  primer
gatcgatcttaattaagttagatctattctgactcacctaggacggtgaccttggtcccagttccgaa

SEQ ID NO: 96 - DNA, 41 bp, artificial; identical to SEQ ID NO: 72
  primer

SEQ ID NO: 97 - DNA, 42 bp, artificial
  primer
gatcgatcggtgaccattgtcccccggccccaataatcaaag

SEQ ID NO: 98 - DNA, 39 bp, artificial
  primer
gatcgatcgtgcactccgcacaggctgtgctgactcagc

SEQ ID NO: 99 - DNA, 73 bp, artificial
  primer
gatcgatcttaattaagttagatctattctgactcacctaggacggtgaccttggtcccgccgccgaacaccg

SEQ ID NO: 100 - DNA, 42 bp, artificial
  primer
gatcgatcgcgcgcactccgaggtccagctggtacagtctgg

SEQ ID NO: 101 - DNA, 34 bp, artificial
  primer
gatcgatcggtgaccagggttcctttgccccagg

SEQ ID NO: 102 - DNA, 45 bp, artificial
  primer
gatcgatcgtgcactccgcactttcttctgagctgactcaggacc

SEQ ID NO: 103 - DNA, 62 bp, artificial; identical to SEQ ID NO: 63
  primer

SEQ ID NO: 104 - DNA, 42 bp, artificial
  primer
gatcgatcgcgcgcactccgaggtccagctggtgcagtctgg

SEQ ID NO: 105 - DNA, 34 bp, artificial
  primer
gatcgatcggtgaccagggtgccctggccccagg

SEQ ID NO: 106 - DNA, 41 bp, artificial
  primer
gatcgatcgtgcactccgcactttcttctgsgctgsctcag

SEQ ID NO: 107 - DNA, 62 bp, artificial; identical to SEQ ID NO: 63
  primer

SEQ ID NO: 108 - protein, 128 aa, artificial
  phage display generated VH or VL region
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGRGTMVTVSS

SEQ ID NO: 109 - protein, 110 aa, artificial
  phage display generated VH or VL region
SSELTQDPAVSVALGQTVRITCQGDSLRSYYASWYQQKPGQAPVLVIYGKNKRPSGIPDRFSGSSSGNTASLTITGAQAEDEADYYCHSRDSSGNHVLFGGGTKLTVLGA

SEQ ID NO: 110 - protein, 128 aa, artificial
  phage display generated VH or VL region
GVQLVQSGAEVKKPGESLTISCKGSGYNFFNYWIGWVRQMPGKGLEWMGIIYPTDSDTRYSPSFQGBVTISVDKSISTAYLQWSSLKASDTAMYYCARSIRYCPGGRCYSGYYGMDVWGQGTMVTVSS

SEQ ID NO: 111 - protein, 110 aa, artificial
  phage display generated VH or VL region
SSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLGA

SEQ ID NO: 112 - protein, 128 aa, artificial
  phage display generated VH or VL region
EVQLVQSGAEVKKPGESLTISCKGSGYNFFNYWIGWVRQMPGKDLEWMGIIYPTDSDTRYSPSFQGQVTISVDKSISTAYLQWSSLKASDTAMYYCARSIRYCPGGRCYSGYYGMDVWGQGTMVTVSS

SEQ ID NO: 113 - protein, 108 aa, artificial
  phage display generated VH or VL region
SSELTQDPAVSVALGQTVRITCRGDSLRNYYASWYQQKPGQAPVLVIYGKNNRPSGIPDRFSGSSSGNTASLTITGAQAEDEADYYCNSRDSSGNHMVGGTKLTVLGA

SEQ ID NO: 114 - protein, 128 aa, artificial
  phage display generated VH or VL region
GVQLVESGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGRGTLVTVSS

SEQ ID NO: 115 - protein, 110 aa, artificial
  phage display generated VH or VL region
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGA

SEQ ID NO: 116 - protein, 128 aa, artificial
  phage display generated VH or VL region
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTLVTVSS

SEQ ID NO: 117 - protein, 108 aa, artificial
  phage display generated VH or VL region
SSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVL

SEQ ID NO: 118 - protein, 128 aa, artificial
  phage display generated VH or VL region
EVQLVQSGAEVKKPGESLTISCKGPGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSS

SEQ ID NO: 119 - protein, 110 aa, artificial; identical to SEQ ID NO: 115
  phage display generated VH or VL region

SEQ ID NO: 120 - protein, 116 aa, artificial
  phage display generated VH or VL region
QVQLVQSGAEVRKPGASVKVSCKTSGYTFRNYDINWVRQAPGQGLEWMGRISGHYGNTDHAQKFQGRFTMTKDTSTSTAYMELRSLTFDDTAVYYCARSQWNVDYWGRGTLVTVSS

SEQ ID NO: 121 - protein, 113 aa, artificial
  phage display generated VH or VL region
NFMLTQPHSVSESPGKTVTISCTRSSGSIASNYVQWYQQRPGSSPTTVIFEDNRRPSGVPDRFSGSIDTSSNSASLTISGLKTEDEADYYCQSFDSTNLVVFGGGTKLTVLGA

SEQ ID NO: 122 - protein, 121 aa, artificial
  phage display generated VH or VL region
EVQLVESGGGVVQPGRSLRLSCAASGFTFSDFAMHWVRQIPGKGLEWLSGLRHDGSTAYYAGSVKGRFTISRDNSRNTVYLQMNSLRAEDTATYYCVTGSGSSGPHAFPVWGKGTLVTVSS

SEQ ID NO: 123 - protein, 112 aa, artificial
  phage display generated VH or VL region
SYVLTQPPSASGTPGQRVTISCSGSNSNIGTYTVNWFQQLPGTAPKLLIYSNNQRPSGVPDRFSGSKSGTSASLAISGLQSEDEADYYCAAWDDSLNGPVFGGGTKVTVLGA

SEQ ID NO: 124 - protein, 127 aa, artificial
  phage display generated VH or VL region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGMGYYGSGGYYPDDAFDVWGQGTMVTVSS

SEQ ID NO: 125 - protein, 110 aa, artificial
  phage display generated VH or VL region
SSELTQDPDVSMALGQTVTISCRGDSLKRFYASWYHQKPGQAPVLVFYGKENRPSGIPDRFSGSDSGDTASLTITGAQAEDEGDYYCHTQDTSARQYVFGSGTKVTVLGA

SEQ ID NO: 126 - protein, 125 aa, artificial
  phage display generated VH or VL region
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGRGTLVTVSS

SEQ ID NO: 127 - protein, 110 aa, artificial
  phage display generated VH or VL region
SSELTQDPAVSVALGQTVRITCQGDSLRNYYASWYQQKPGQAPLLVMFGKNNRPSEIPGRFSGSSSGNTASLTITGAQAEDEADYYCNSRDRNSHQWVFGGGTKLTVLGA

SEQ ID NO: 128 - protein, 121 aa, artificial
  phage display generated VH or VL region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASSPYSSRWYSFDPWGQGTMVTVSS

SEQ ID NO: 129 - protein, 108 aa, artificial
  phage display generated VH or VL region
SYELTQPPSVSVSPGQTATITCSGDDLGNKYVSWYQQKPGQSPVLVIYQDTKRPSGIPERFSGSNSGNIATLTISGTQAVDEADYYCQVWDTGTVVFGGGTKLTVLGA

SEQ ID NO: 130 - protein, 125 aa, artificial
  phage display generated VH or VL region
QVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGKGTLVTVSS

SEQ ID NO: 131 - protein, 111 aa, artificial
  phage display generated VH or VL region
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHHWVFGGGTKVTVLGA

SEQ ID NO: 132 - protein, 119 aa, artificial
  phage display generated VH or VL region
QVQLVESGGGLVKPGGSLRLSCAASGFTFSSHTMNWVRQAQGKGLEWVSSISGSGRYIYYSDSVKGRFTISRDAAKNSLYLQMNNLRAEDTAVYYCTRAKFGDYLFDSWGQGTLVTVSS

SEQ ID NO: 133 - protein, 112 aa, artificial
  phage display generated VH or VL region
NFMLTQPHSVSQSPGKTVTISCTRSSGRIASNFVQWYQQRPGSAPTTVIYEDNRRPSGVPDRFSGSIDSSSNSASLTISGLKTEDEADYYCQSYDARYQVFGTGTKVTVLGA

SEQ ID NO: 134 - protein, 122 aa, artificial
  phage display generated VH or VL region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASPVPPWADWYYFDYWGRGTMVTVSS

SEQ ID NO: 135 - protein, 113 aa, artificial
  phage display generated VH or VL region
QAVLTQPSSVSGAPGQRVTISCTGSRSNFGAGYDVHWYQQFPGTAPKLLIYGNTNRPSGVPDRFSGSRSGTSASLAITGLQAEDEADYYCQSYDSNLSGSVFGGGTKVTVLGA

SEQ ID NO: 136 - protein, 125 aa, artificial
  phage display generated VH or VL region
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGKGTLVTVSS

SEQ ID NO: 137 - protein, 111 aa, artificial
  phage display generated VH or VL region
SSELTQDPAVSVALGQTVRITCQGDSLRNYYASWYQQKPGQAPVLVLYSKNSRPSGVPDRFSGSSSGTTASLTISGAQAEDEADYYCNSRDTSGDLRWVFGGGTKLTVLGA

SEQ ID NO: 138 - protein, 125 aa, artificial
  phage display generated VH or VL region
EVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGQGTLVTVSS

SEQ ID NO: 139 - protein, 110 aa, artificial
  phage display generated VH or VL region
SSELTQDPAVSVALGQTVRITCQGDSLRNYYASWYQQKPGQAPLLVMFGKNNRPSEIPGRFSGSSSGNTASLTITGAQAEDEADYYCNSRDSNSHQWVFGGGTKLTVLGA

SEQ ID NO: 140 - protein, 125 aa, artificial
  phage display generated VH or VL region
QVQLVQSGAEVKKPGASVKVSCKASGYSFTNYGLNWVRQAPGQGLEWMGWISPYTGYTNYAQKFQGRVTMTSDKSTSTAYMDLRSLRSDDTAIYYCAREIFSHCSGGSCYPFDYWGQGTLVTVSS

SEQ ID NO: 141 - protein, 112 aa, artificial
  phage display generated VH or VL region
SSELTQDPAVSVALGQTVRITCQGDSLRSYYASWYQQKPGQAPLLVIYGRNNRPSGIPDRFSGSSSGNTASLTITGAQAEDEADYYCNSRDSSTNHGNWVFGGGTQLTVLSA

SEQ ID NO: 142 - protein, 125 aa, artificial
  phage display generated VH or VL region
QVQLVQSGAEVKKPGASVKVSCKASGYSFTNYGLNWVRQAPGQGLEWMGWISPYTGYTNYAQKFQGRVTMTTDKSTSTAYMDLRSLRSDDTAVYYCAREIFSHCTGGSCYPFDSWGRGTMVTVSS

SEQ ID NO: 143 - protein, 111 aa, artificial
  phage display generated VH or VL region
SSELTQDPAVSVALGQTVRITCQGDSLRSYYASWYQQKPGQAPVLVIYGKNNRPSGIPDRFSGSSSGNTASLTITGAQAEDEADYYCNSRDSSGNLNWVFGGGTQLTVLSA

SEQ ID NO: 144 - protein, 109 aa, homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGQGTMVTVSS

SEQ ID NO: 145 - protein, 109 aa, homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGRGTLVTVSS

SEQ ID NO: 146 - protein, 109 aa, homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGQGTLVTVSS

SEQ ID NO: 147 - protein, 109 aa, homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARWGRGTLVTVSS

SEQ ID NO: 148 - protein, 109 aa, homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARWGQGTLVTVSS

SEQ ID NO: 149 - protein, 109 aa, homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTMVTVSS

SEQ ID NO: 150 - protein, 109 aa, homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARWGQGTLVTVSS

SEQ ID NO: 151 - protein, 108 aa, homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 152 - protein, 111 aa, homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNLVVFGGGTKLTVL

SEQ ID NO: 153 - protein, 108 aa, homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGTGTKVTVL

SEQ ID NO: 154 - protein, 110 aa, homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGTGTKVTVL

SEQ ID NO: 155 - protein, 105 aa, homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTAFGGGTKLTVL

SEQ ID NO: 156 - protein, 108 aa, homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNFGTGTKVTVL

SEQ ID NO: 157 - protein, 109 aa, homo sapiens
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFGTGTKVTVL

(19) United States
(12) Patent Application Publication
(10) Pub. No.: US 2004/0202655 A1
(43) Pub. Date: Oct. 14, 2004

(54) Antibodies to IGF-I receptor for the treatment of cancers

(76) Inventors: Phillip A. Morton, Chesterfield, MO (US); John A. Arbuckle, Brentwood, MO (US); Karen S. Bailey, Maryville, MO (US); Peter J. Nicastro, Overland, MO (US); Herbert A. Runnels, Chesterfield, MO (US)

Correspondence Address: Pharmacia Corporation, Global Patent Department, P. O. Box 1027, Mail Zone MC5, St. Louis, MO, 63141, US

(21) Appl. No.: 10/800,197

(22) Filed: Mar. 12, 2004

Related U.S. Application Data
(60) Provisional application No. 60/455,094, filed on Mar. 14, 2003.

(51) Int. Cl. A61K 39/395; C07K 16/28

(52) U.S. Cl. 424/143.1; 530/388.22

(57) ABSTRACT

Antibodies specific for Insulin-like growth factor I receptor (IGF-IR) are provided. The antibodies and fragments thereof may block binding of IGF-I to IGF-IR. Antagonist antibodies can be employed to block binding of IGF-I to IGF-IR or substantially inhibit IGF-IR activation. The IGF-IR antibodies may be included in pharmaceutical compositions, articles of manufacture, or kits. Methods of treating cancer, inflammation, and pathological liver conditions, using the IGF-IR antibodies are also provided.

The present application claims priority under Title 35, United States Code, §119 to U.S. Provisional application Serial No. 60/455,094, filed Mar. 14, 2003, which is incorporated by reference in its entirety as if written herein.

### FIELD OF THE INVENTION

This application relates to insulin-like growth factor I (IGF-I) receptor antibodies, particularly antagonists of IGF-I and IGF-II binding to IGF-I receptor. The application also relates to the use of the antibodies in therapy or diagnosis of particular pathological conditions in mammals, including cancer.

### BACKGROUND OF THE INVENTION

Insulin-like growth factor I (IGF-I; also called somatomedin-C) is a member of a family of related polypeptide hormones that also includes insulin, insulin-like growth factor II (IGF-II) and more distantly nerve growth factor. Each of these hormone growth factors has a cognate receptor to which it binds with high affinity, but some may also bind (albeit with lower affinity) to the other receptors as well (for review, see Rechler and Nissley, Ann. Rev. Physiol. 47:425-42 (1985). IGF-I stimulates cell differentiation and cell proliferation, inhibits apoptosis, and is required by most mammalian cell types for sustained proliferation. These cell types include, among others, human diploid fibroblasts, epithelial cells, smooth muscle cells, T lymphocytes, neural cells, myeloid cells, chondrocytes, osteoblasts, and bone marrow stem cells. For a review of the wide variety of cell types for which IGF-I/IGF-I receptor interaction mediates cell proliferation, see Goldring et al., Eukar. Gene Express., 1:31-326 (1991).

The first step in the transduction pathway leading to IGF-I-stimulated cellular proliferation or differentiation is binding of IGF-I or IGF-II (or insulin at supraphysiological concentrations) to the IGF-I receptor. The IGF-I receptor is composed of two types of subunits: an alpha subunit (a 130-135 kDa protein that is entirely extracellular and functions in ligand binding) and a beta subunit (a 95-kDa transmembrane protein, with transmembrane and cytoplasmic domains). The IGF-IR belongs to the family of tyrosine kinase growth factor receptors (Ullrich et al., Cell 61: 203-212, 1990), and is structurally quite similar to the insulin receptor (Ullrich et al., EMBO J 5: 2503-2512, 1986). Additional family members include the insulin-related receptor and so-called hybrid receptors comprised of one subunit each from the IGF-1R and insulin receptor. The IGF-IR is initially synthesized as a single chain proreceptor polypeptide, which is further post-translationally modified by glycosylation, proteolytic cleavage by preprotein convertases, and disulfide bonding to assemble a mature 460-kDa heterotetramer comprised of two extracellular 130-135 kD alpha subunits and two transmembrane 90-95 kDa beta subunits (Massague and Czech, J. Biol. Chem. 257:5038-6045, 1982). The beta subunit(s) possess intrinsic receptor tyrosine kinase activity required for all IGF-1R functions (Kato et al., Mol. Endocrinol. 8:40-50, 1994), whereas the alpha subunits are entirely extracellular and possess the ligand binding activity of the IGF-1R.

In vivo, serum levels of IGF-I are dependent upon the presence of pituitary growth hormone (GH). Although the liver is a major site of GH dependent IGF-I synthesis, a large number of extrahepatic tissues also produce IGF-I (Daughaday and Rotwein, Endocrine Rev. 10:68-91 (1989). A variety of neoplastic tissues may also produce IGF-I (Werner and LeRoith, Adv. Cancer Res. 68:183-223 (1996). Thus IGF-I may act as a regulator of normal and abnormal cellular proliferation via autocrine or paracrine, as well as endocrine mechanisms. IGF-I and IGF-II bind to IGF binding proteins (IGFBPs) in vivo. Upon binding to IGFs the IGFBPs either transport IGFs through the circulation or they may protect IGFs from proteolytic cleavage and inactivation. The availability of free IGF for interaction with the IGF-IR is modulated by the IGFBPs. For a review of IGFBPs and IGF-I, see Grimberg et al., J. Cell. Physiol. 183: 1-9, 2000.

There is considerable evidence for a role for IGF-I and/or IGF-IR in the maintenance of tumor cells in vitro and in vivo. IGF-IR levels are elevated in tumors of lung (Kaiser et al., J. Cancer Res. Clin Oncol. 119: 665-668, 1993; Moody et al., Life Sciences 52: 1161-1173, 1993; Macauley et al., Cancer Res., 50: 2511 2517, 1990), breast (Pollak et al., Cancer Lett. 38: 223-230, 1987; Foekens et al., Cancer Res. 49: 7002-7009, 1989; Cullen et al., Cancer Res. 49: 7002-7009, 1990; Arteaga et al., J. Clin. Invest. 84: 1418-1423, 1989), prostate (Hellawell et al., Cancer Res. 62:2942-2950, 2002) and colon (Remaole-Bennet et al., J. Clin. Endocrinol. Metab. 75: 609-616, 1992; Guo et al., Gastroenterol.102: 1.101-1108, 1992). In addition to wild-type IGF-1R, transformed cells and tumor cells may also express so-called hybrid receptors comprised of a single alpha and beta subunit each from the IGF-1R and the insulin receptor (Soos et al., Biochem. J. 270:383-390, 1990) and Bailyes et al., Biochem. J. 327:209-215, 1997). Enhanced tyrosine phosphorylation of the IGF-1R has been detected in human medulloblastoma (Del Valle et al., Clin. Cancer Res. 8:1822-1830, 2002) and in human breast cancer (Resnik et al., Cancer Res. 58:1159-1164, 1998). Deregulated expression of IGF-I in prostate epithelium leads to neoplasia in transgenic mice (DiGiovanni et al., Proc. Natl. Acad. Sci. USA 97: 3455-60, 2000). In addition, IGF-I appears to be an autocrine stimulator of human gliomas (Sandberg-Nordqvist et al., Cancer Res. 53: 2475-2478, 1993), while IGF-I stimulated the growth of fibrosarcomas that overexpressed IGF-IR (Butler et al., Cancer Res. 58: 3021-27, 1998). Furthermore, individuals with “high normal” levels of IGF-I have an increased risk of common cancers compared to individuals with IGF-I levels in the “low normal” range (Rosen et al., Trends Endocrinol. Metab. 10: 136-41, 1999). Many of these tumor cell types respond to IGF-I with a proliferative signal in culture (Nakanishi et al., J. Clin. Invest. 82: 354 359, 1988; Freed et al., J. Mol. Endocrinol. 3: 509-514, 1989), and autocrine or paracrine loops for proliferation in vivo have been postulated (LeRoith et al., Endocrine Revs. 16: 143-163, 1995; Yee et al., Mol. Endocrinol. 3: 509-514, 1989). Over-expression of IGF-IR has been found in colorectal carcinomas (Weber et al., Cancer 95:2086-2095, 2002). For a review of the insulin-like growth factor system as a therapeutic target in colorectal cancer see Hassan A. B. & Macaulay.(Anals of Oncology 13:349-356, 2002). For a review of the role IGF-I/IGF-I receptor interaction plays in the growth of a variety of human tumors see Macaulay, Br. J. Cancer, 65: 311-320, 1992 and Werner and LeRoith, Adv. Cancer Res. 68:183-223, 1996.

A number of approaches to interfere with the activity and/or expression of the IGF-1R have been employed in vitro and in vivo to demonstrate the critical role of this receptor in tumor cell biology. Using antisense expression vectors or antisense oligonucleotides to the IGF-IR RNA, it has been shown that interference with IGF-IR leads to inhibition of IGF-I-mediated or IGF-II-mediated cell growth (see, e.g., Wraight et al., Nat. Biotech. 18: 521 -526, 2000). The antisense strategy was successful in inhibiting cellular proliferation in several normal cell types and in human tumor cell lines. Growth can also be inhibited using cyclic peptide analogues of IGF-I (Pietrzkowski et al., Cell Growth & Dif. 3: 199-205, 1992; and Pietrzkowski et al., Mol. Cell. Biol., 12: 3883-3889, 1992), or a vector expressing an antisense RNA to the IGF-I RNA (Trojan et al., Science 259: 94-97, 1992). In addition, antibodies to IGF-IR, especially a mouse IgG1 monoclonal antibody designated αIR3 (Kull et al., J. Biol. Chem. 258:6561-6566, 1983) can inhibit proliferation of a number of tumor cell lines in vitro and in vivo (Arteaga et al., Breast Cancer Res. Treat., 22:101-106, 1992; Rohlik et al., Biochem. Biophys. Res. Commun. 149:276-281; Arteaga et al., J. Clin. Invest. 84:1418-1423, 1989; Kalebic et al., Cancer Res. 54: 5531-5534, 1994). Furthermore, single-chain antibodies against IGF-1R have also been shown to inhibit growth of MCF-7 human breast cancer cells in xenografts models (Li et al., Cancer Immunol. Immunother. 49:243-252, 2000) and to lead to down-regulation of cell surface receptors (Sachdev et al., Cancer Res. 63: 627-635 (2003). In an alternative strategy, interference with IGF-1R kinase activity by co-expression in cells of dominant-negative mutants of the IGF-1R (Prager et al., Proc. Natl. Acad. Sci. U.S.A. 91: 2181-2185, 1994; Li et al., J. Biol. Chem., 269: 32558-32564, 1994 and Jiang et al., Oncogene 18: 6071-77, 1999), can also reverse the transformed phenotype, inhibit tumorigenesis, and induce loss of the metastatic phenotype.

IGF-IR activity also contributes to the regulation of apoptosis. Apoptosis, also known as programmed cell death, is involved in a wide variety of developmental processes, including lymphocyte maturation and regulation and nervous system maturation. In addition to its role in development, apoptosis also has been implicated as an important cellular safeguard against tumorigenesis (Williams, Cell 65: 1097-1098, 1991; Lane, Nature 362: 786-787, 1993). Suppression of the apoptotic program by a variety of genetic lesions may contribute to the development and progression of malignancies.

IGF-I protects hematopoietic cells from apoptosis induced by withdrawal of IL-3 (Rodriguez-Tarduchy, G. et al., J. Immunol. 149: 535 540, 1992), and from serum withdrawal in Rat-1/mycER cells (Harrington, E., et al., EMBO J. 13: 3286-3295, 1994). The anti-apoptotic function of IGF-I is important in the post-commitment stage of the cell cycle and also in cells blocked in cell cycle progression by etoposide or thymidine. The demonstration that c-myc-driven fibroblasts are dependent on IGF-I for their survival suggests that there is an important role for the IGF-IR in the maintenance of tumor cells by specifically inhibiting apoptosis, a role distinct from the proliferative effects of IGF-I or IGF-IR. This would be similar to a role thought to be exerted by other anti-apoptotic genes, such as Bcl-2, in promoting tumor cell survival (McDonnell et al., Cell 57: 79-88, 1989; Hockenberry et al., Nature 348: 334-336, 1990).

The protective effects of IGF-I on apoptosis are dependent upon having IGF-IR present on cells to interact with IGF-I (Resnicoff et al., Cancer Res. 55: 3739-3741, 1995). Support for an anti-apoptotic function of IGF-IR in the maintenance of tumor cells was also provided by a study using antisense oligonucleotides to the IGF-IR that identified a quantitative relationship between IGF-IR levels, the extent of apoptosis and the tumorigenic potential of a rat syngeneic tumor (Resnicoff et al., Cancer Res. 55: 3739-3741, 1995). An over-expressed IGF-IR has been found to protect tumor cells in vitro from etoposide-induced apoptosis (Sell et al., Cancer Res. 55: 303-306, 1995) and, even more dramatically, that a decrease in IGF-IR levels below wild type levels caused massive apoptosis of tumor cells in vivo (Resnicoffet al., Cancer Res. 55: 24632469, 1995).

Potential strategies for inducing apoptosis or for inhibiting cell proliferation associated with increased IGF-I, increased IGF-II, and/or increased IGF-IR receptor levels include suppressing IGF-I levels or IGF-II levels or preventing the binding of IGF-I to the IGF-IR. For example, the long acting somatostatin analogue octreotide has been employed to reduce IGF synthesis and/or secretion. Soluble IGF-IR has been used to induce apoptosis in tumor cells in viva and inhibit tumorigenesis in an experimental animal system (D'Ambrosio et al., Cancer Res. 56: 4013-20, 1996). In addition, IGF-IR antisense oligonucleotides, peptide analogues of IGF-I, and antibodies to IGF-IR have been used to decrease IGF-I or IGF-IR expression (see supra). However, none of these compounds has been suitable for long-term administration to human patients. In addition, although IGF-I has been administered to patients for treatment of short stature, osteoporosis, decreased muscle mass, neuropathy or diabetes, the binding of IGF-I to IGFBPs has often made treatment with IGF-I difficult or ineffective.

Accordingly, in view of the roles that IGF-I and IGF-IR have in such disorders as cancer and other proliferative disorders when IGF-I and/or IGF-IR are over-expressed, it would be desirable to generate antibodies to IGF-IR that could inhibit expression and/or activity of IGF-IR. Although anti-IGF-IR antibodies have been reported present in certain patients with autoimmune diseases, none of these antibodies has been purified and none has been shown to be suitable for inhibiting IGF-I activity for diagnostic or clinical procedures. See, e.g., Thompson et al., Pediat. Res. 32: 455 459, 1988; Tappy et al., Diabetes 37: 1708-1714, 1988; Weightman et al., Autoimmunity 16:251-257, 1993; Drexhage et al., Nether. J. of Med. 45:285-293, 1994. Additionally, monoclonal antibodies against the IGF-1R have been reported with can stimulate cell proliferation (Xiong et al., Proc. Natl. Acad. Sci. USA 89:5356-5360, 1992).

WO 02/053596 discloses hybridomas expressing anti-IGF-1R IgG antibodies obtained using XENOMICE™ and methods of treating cancers using such.

Thus, it would be desirable to obtain high-affinity human anti-IGF-IR antibodies that could be used to treat diseases in humans. Herein we disclose fully human antibodies to IGF-1R obtained using phage-display libraries and methods of using the antibodies to treat animal cancers.

### BRIEF DESCRIPTION OF THE DRAWINGS

FIGS. 1a-s show alignments of the amino acid sequences of the light and heavy regions of scFvs PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 IGF-1R scFv antibodies to the germline sequence. Differences between query sequence and the first germline sequence are bolded and underlined. CDR sequences are highlighted in gray boxes.

FIGS. 2a & 2b shows the inhibition of IGF-I binding to NIH 3T3 fibroblasts expressing the human IGF-1R by IGF-IR antibodies 7A6, 9A2, and 12A1 and inhibition of IGF-II binding to NIH 3T3 fibroblasts expressing the human IGF-1R by IGF-IR antibodies 7A4, 8A1, and 9A2, respectively.

FIG. 3 shows that IGF-IR antibodies 8A1, 9A2, and 11A4 do not inhibit binding of insulin to CHO cells expressing the human insulin receptor.

FIG. 4 shows that several of the IGF-1R antibodies of the invention do not block insulin receptor activation in response to ligand binding.

FIG. 5 shows saturable and specific binding of IGF-IR antibodies 8A1 and 11A4 to NIH 3T3-fibroblasts expressing the human IGF-1R.

FIG. 6 shows that IGF-IR antibodies 8A1, 9A2, and 11A4 inhibit IGF-1-driven cell proliferation of NIH 3T3-fibroblasts expressing the human IGF-1R.

FIG. 7 shows minimal or no ability of the IGF-1R antibodies of the invention to induce tyrosine phosphorylation of IGF-1R on NIH 3T3-fibroblasts expressing the human IGF-1R by Western blot analysis.

FIG. 8 shows minimal or no ability of the IGF-1R antibodies of the invention to induce tyrosine phosphorylation of the IGF-1R on NIH 3T3 -fibroblasts expressing the human IGF-1R using an ELISA format.

FIG. 9 shows the relative ability of IGF-1R antibodies 7A2, 7A4, 8A1, 11A5, 11A11, and 11A12 to inhibit IGF-1 driven tyrosine. phosphorylation of the kinase domain of the IGF-1R.

FIG. 10 shows that IGF-1R antibodies 8A1, 9A2, and 11A4 decrease the amount of surface IGF-1R expression over time on NIH 3T3-fibroblasts expressing the human IGF-1R by FACS.

FIG. 11 shows that IGF-1R antibodies 8A1 and 11A4 can decrease total cell-associated IGF-1R expression over time on NIH 3T3-fibroblasts expressing the human IGF-1R by Western blot analysis.

FIG. 12 shows that IGF-1R antibodies 8A1, 9A2, and 11A4 can decrease the level of surface IGF-1R on NIH-3T3 cells expressing the human IGF-1R (receptor down-regulation).

FIG. 13 shows that IGF-1R antibodies 8A1, 9A2, and l 1A4 can decrease the level of IGF-1R expressed by AA549 NSCLC cells (receptor down-regulation).

FIG. 14 shows the rate of intracellular accumulation of IGF-1R by indirectly measuring the intracellular accumulation of [¹²⁵I]-labeled monoclonal antibodies 8A1 , 9A2, and 11A4 of the invention compared to [¹²⁵I]-labeled IGF-1 using human prostate cancer cells expressing the human IGF-1R.

FIG. 15 shows that IGF-1R antibodies of the invention bind to the same or different epitopes of the IGF-1R on NIH 3T3 fibroblasts expressing the human IGF-1R.

FIG. 16 shows that IGF-1R antibodies 8A1, 9A2, and 11A4 have distinct binding epitopes on the IGF-1R.

FIG. 17 shows that IGF-IR antibodies 8A1 and 11A4 inhibit tumor growth and decrease IGF-1R expression on NIH 3T3-fibroblasts expressing the human IGF-1R.

FIG. 18 shows that IGF-1R antibody 8A1 inhibits tumor growth and decreases tumor IGF-1R expression on NIH 3T3-fibroblasts expressing the human IGF-1R.

FIG. 19 shows that IGF-1R antibody 11A4 inhibits tumor growth and decreases tumor IGF-1R expression on NIH 3T3-fibroblasts expressing the human IGF-1R.

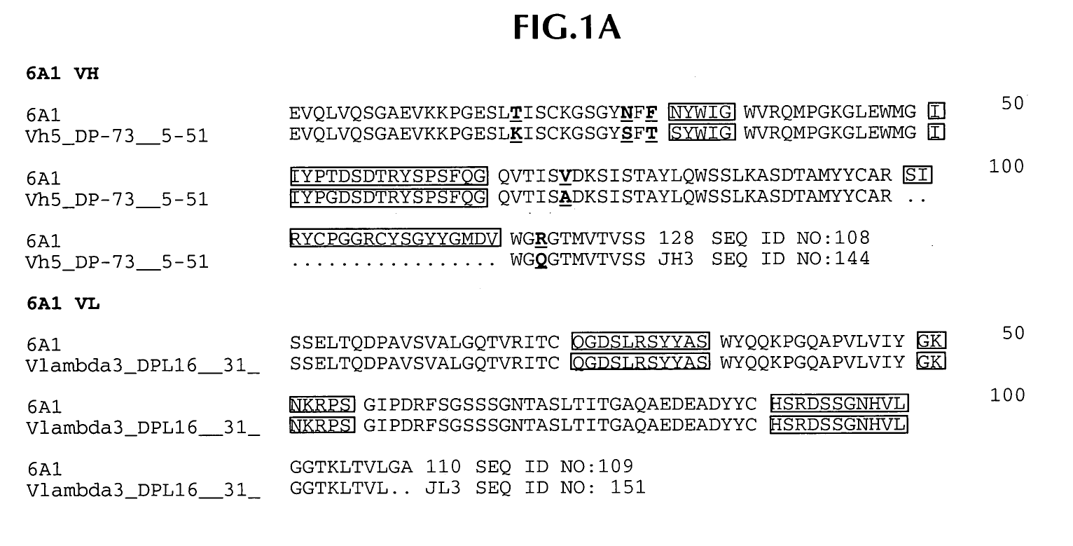

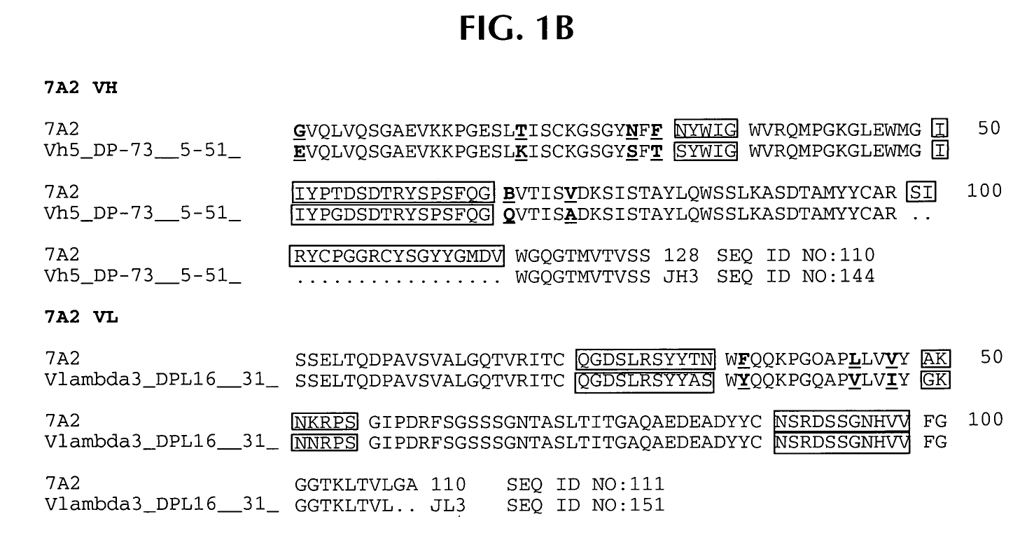

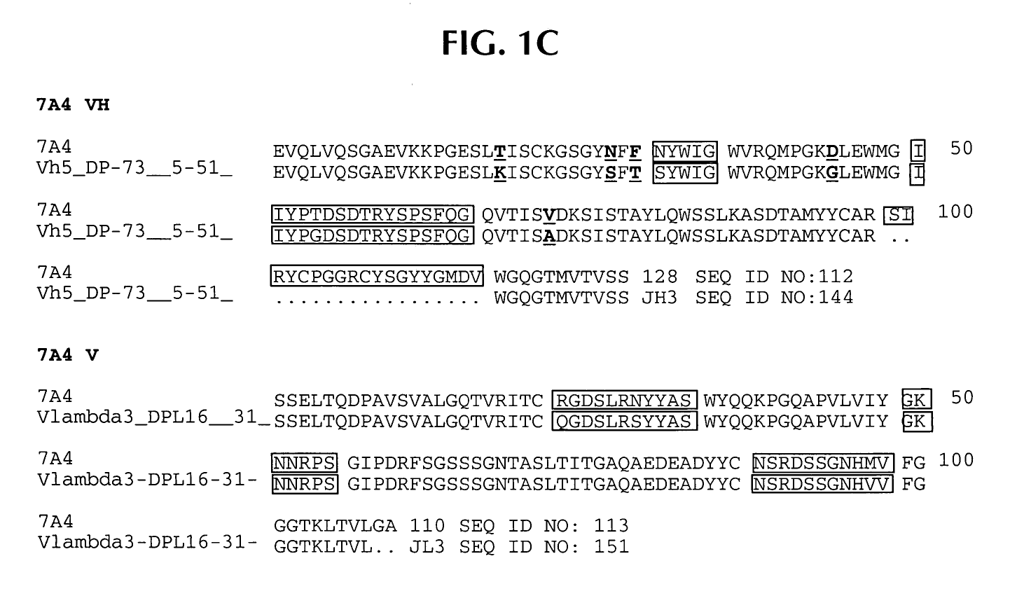

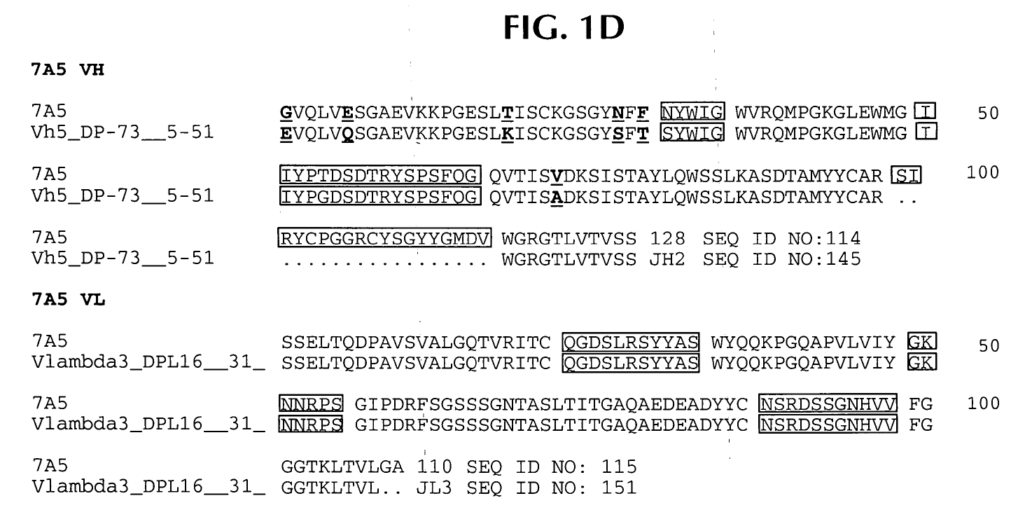

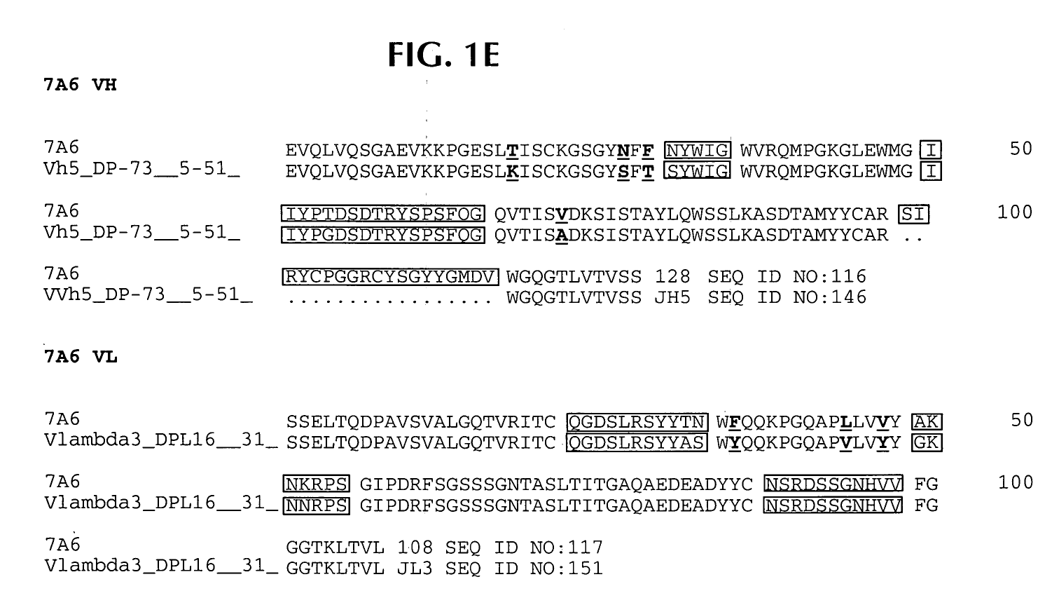

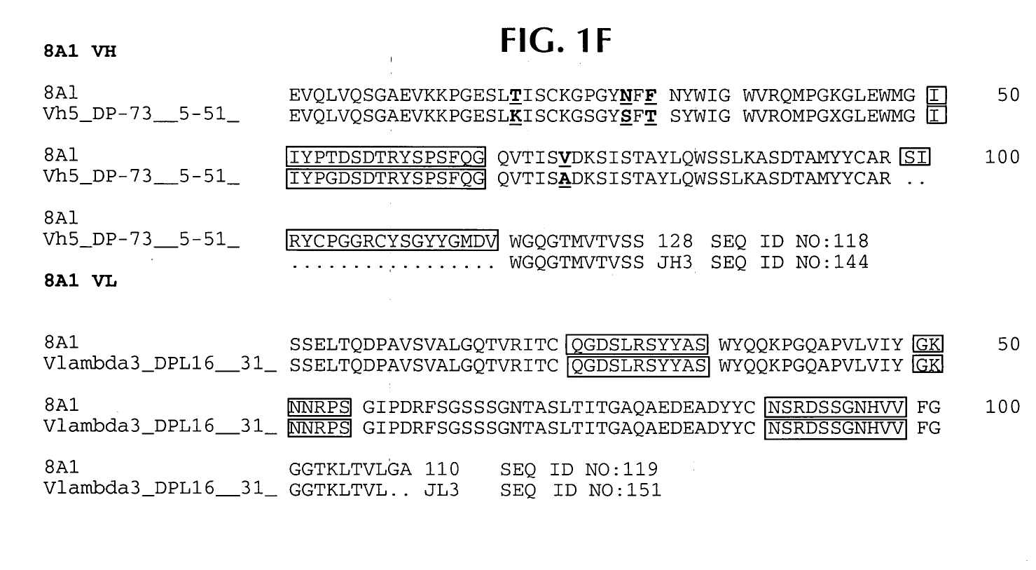

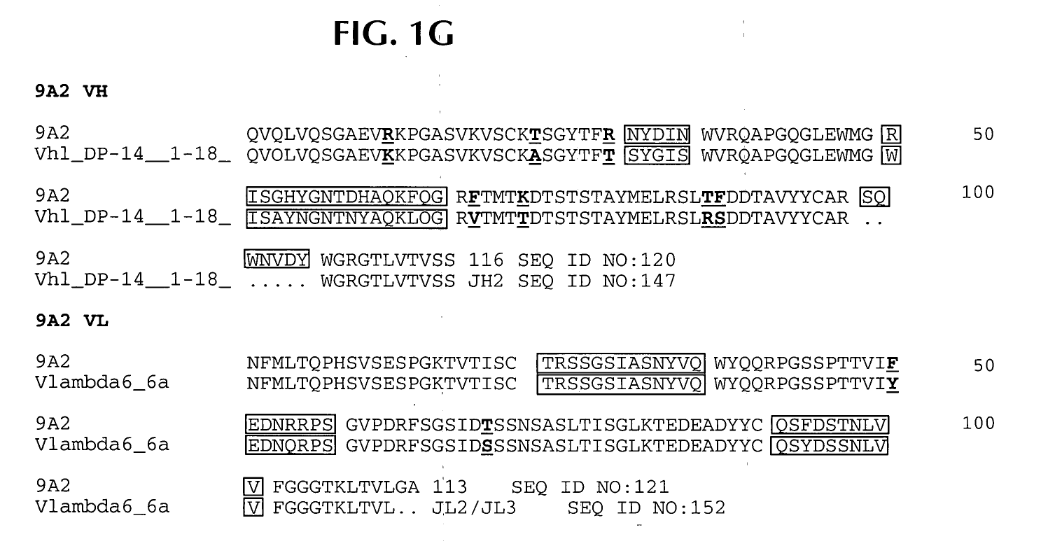

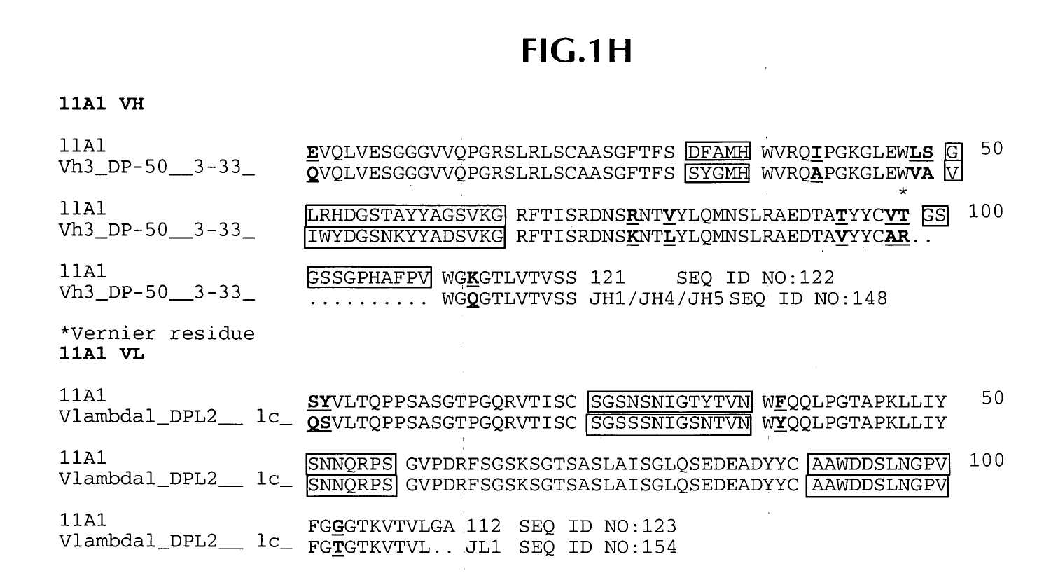

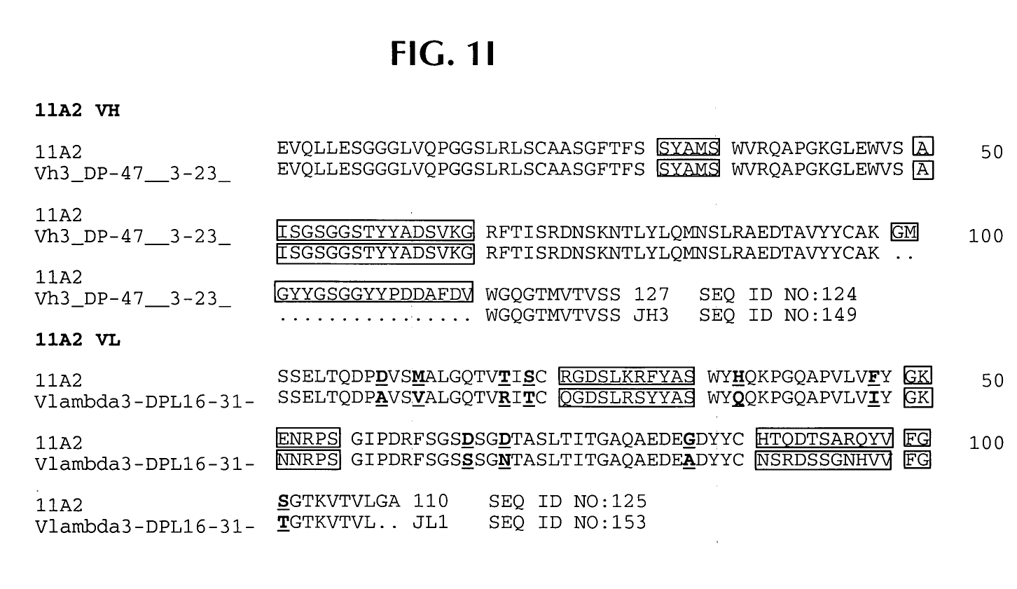

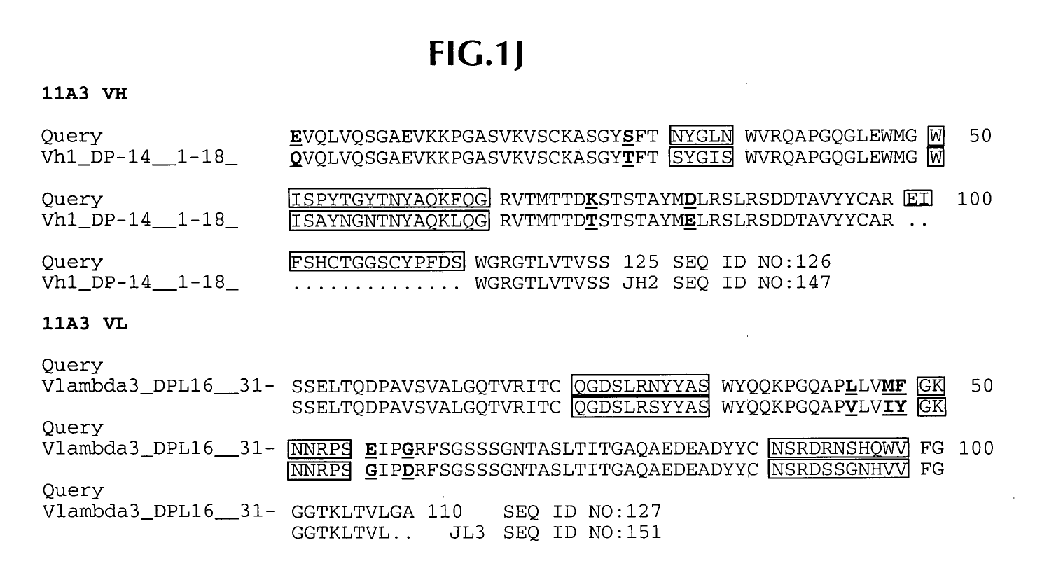

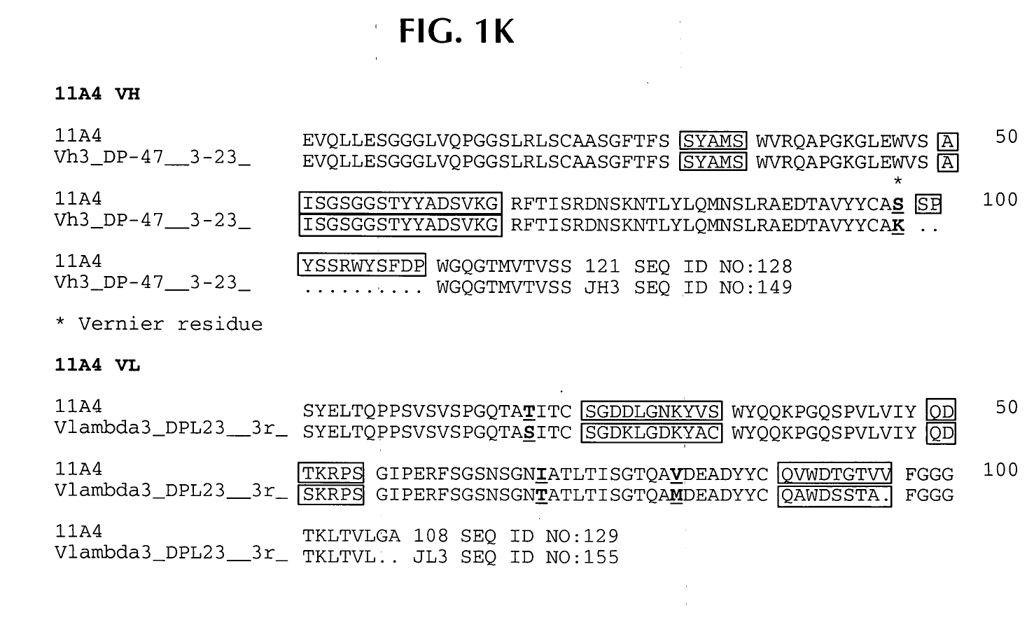

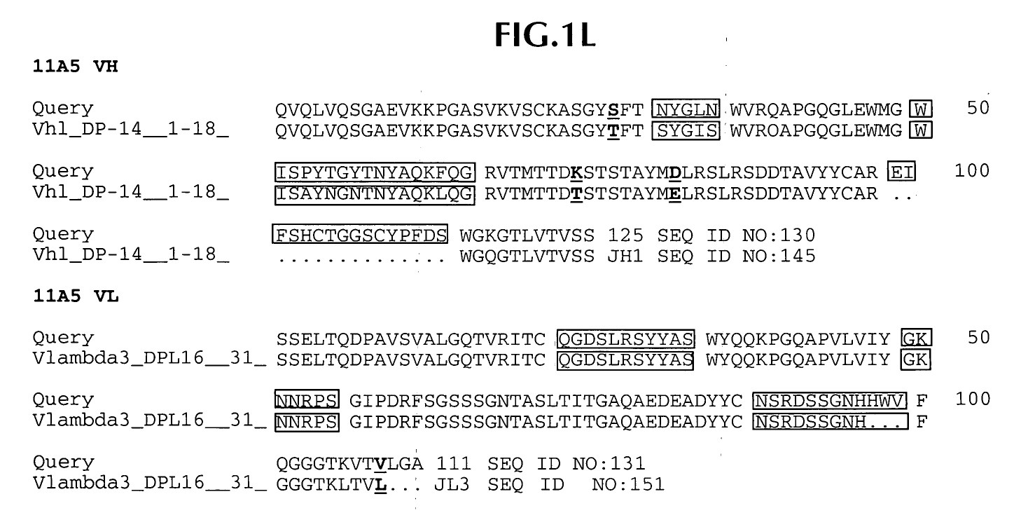

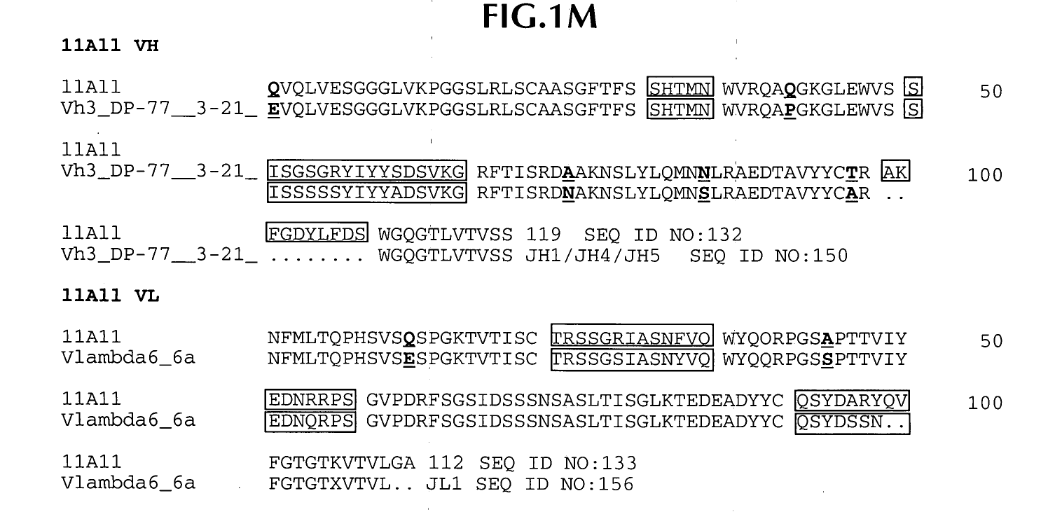

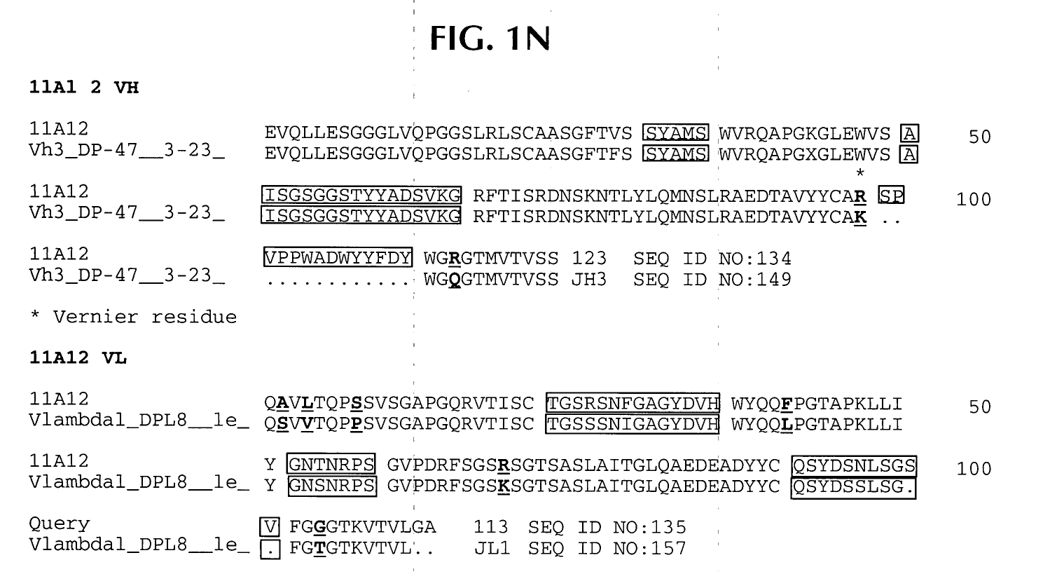

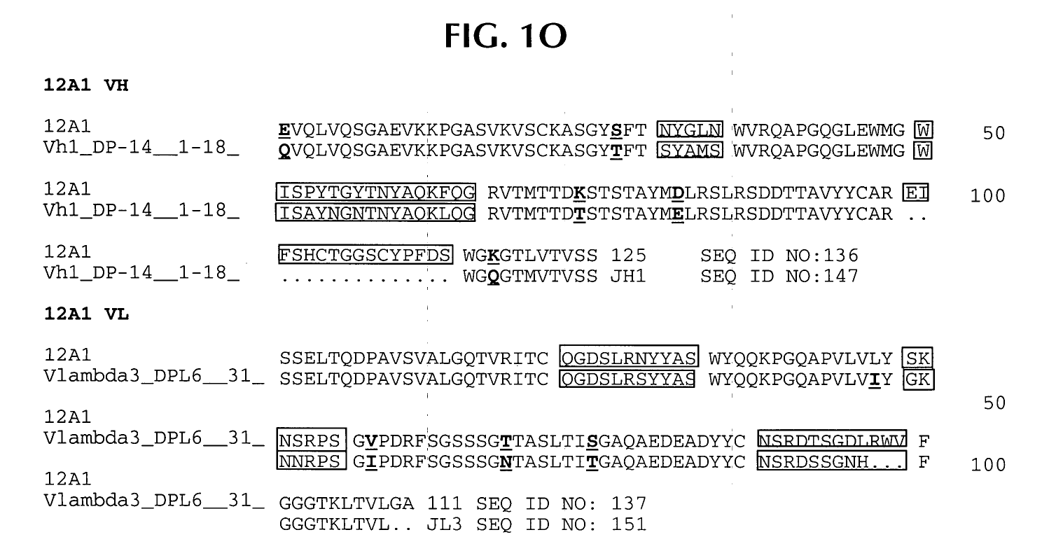

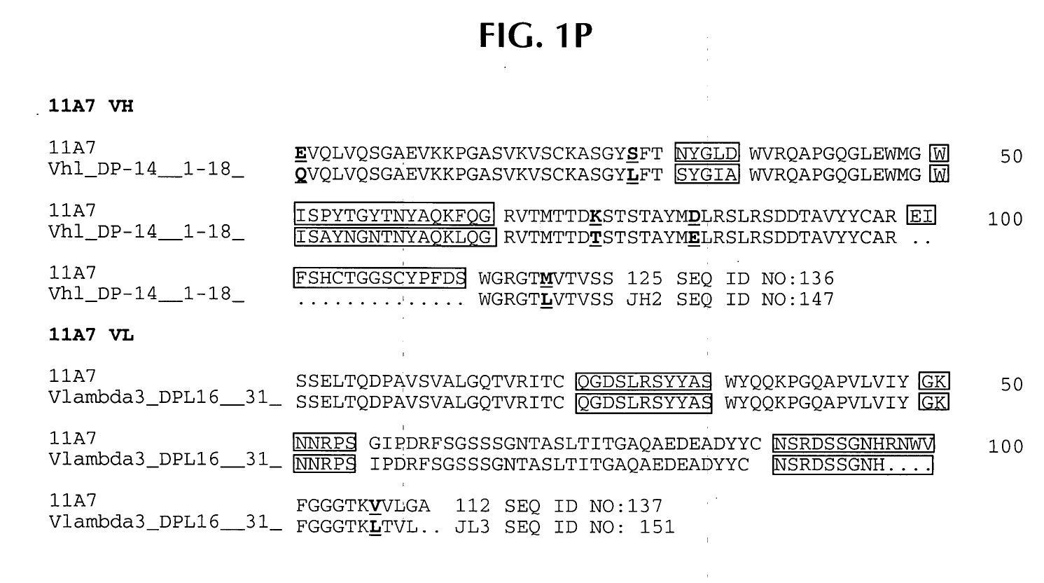

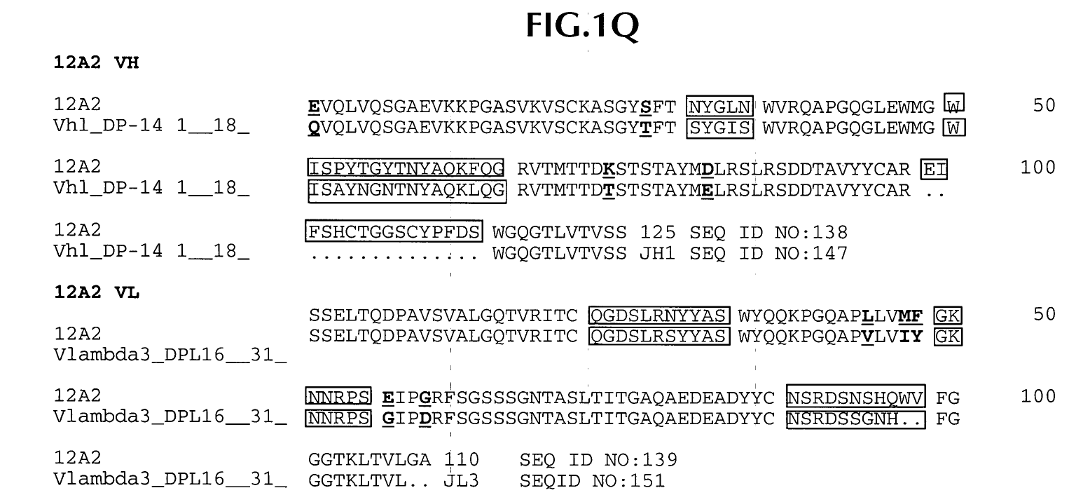

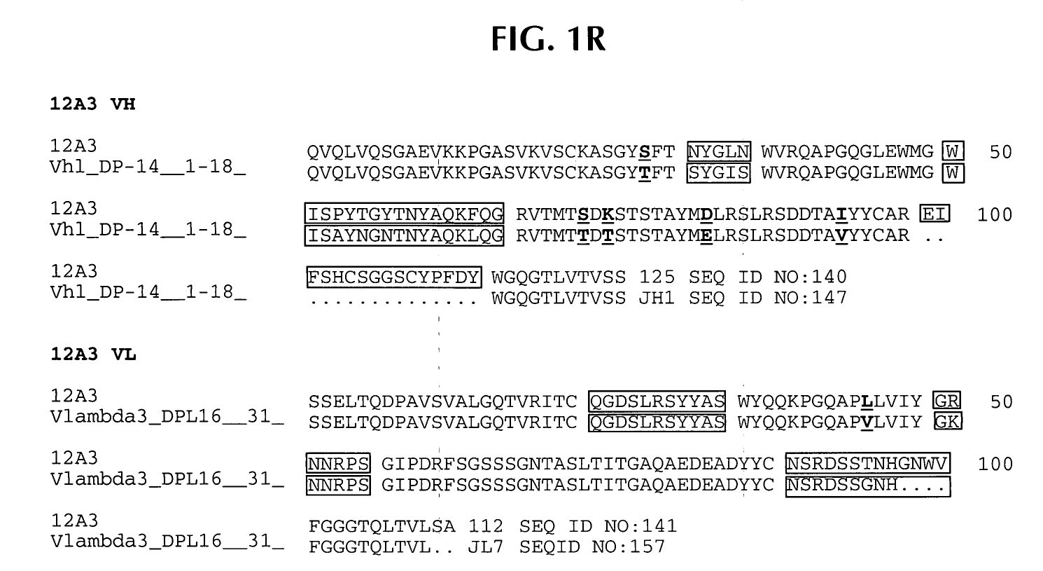

### SUMMARY OF THE INVENTION

The present invention provides an isolated antibody, or antigen-binding portion thereof, that binds IGF-IR, preferably one that binds to mouse, rat, primate and human IGF-IRs, and more preferably one that is a human antibody. The invention provides IGF-IR antibodies that inhibit the binding of IGF-I and IGF-II to IGF-IR, and also provides IGF-IR antibodies that activate IGF-IR tyrosine phosphorylation.

The invention provides a pharmaceutical composition comprising the antibody and a pharmaceutically acceptable carrier. The pharmaceutical composition may further comprise another component, such as an anti-tumor agent or an imaging reagent.

Diagnostic and therapeutic methods are also provided by the invention. Diagnostic methods include a method for diagnosing the presence or location of an IGF-IR-expressing tissue using an IGF-IR antibody. A therapeutic method comprises administering the antibody to a subject in need thereof, preferably in conjunction with administration of another therapeutic agent.

The invention provides an isolated cell line, such as a hybridoma, that produces an IGF-IR antibody.

The invention also provides nucleic acid molecules encoding the heavy and/or light chain or antigen-binding portions thereof of an IGF-IR antibody.

The invention provides vectors and host cells comprising the nucleic acid molecules, as well as methods of recombinantly producing the polypeptides encoded by the nucleic acid molecules.

Non-human transgenic animals that express the heavy and/or light chain or antigen-binding portions thereof of an IGF-IR antibody are also provided. The invention also provides a method for treating a subject in need thereof with an effective amount of a nucleic acid molecule encoding the heavy and/or light chain or antigen-binding portions thereof of a IGF-IR antibody.

### DETAILED DESCRIPTION OF THE INVENTION

Definitions and General Techniques

Unless otherwise defined herein, scientific and technical terms used-in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Using Antibodies: A Laboratory Manual Cold Spring Harbor Laboratory Press, Cold Spring Harbor, N.Y. (1999), which are incorporated herein by reference.

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

As used herein, the terms “insulin-like growth factor I” or “IGF-I” and “insulin-like growth factor II” or “IGF-II” refer to a growth factor typically having A through D domains Fragments of IGF-I or IGF-II constitute IGF-I or IGF-II with fewer domains and variants of IGF-I or IGF-II may have some of the domains of IGF-I or IGF-II repeated; both are included if they still retain their respective ability to bind a IGF-I receptor. The terms “IGF-I” and “IGF-II” include growth factor from humans and any non-human mammalian species, and in particular human IGF-I and IGF-II. The terms as used herein include mature, pre, pre-pro, and pro forms, purified from a natural source, chemically synthesized or recombinantly produced. Human IGF-I is encoded by the cDNA sequence published by Jensen M. et al. (Nature 306:609-611, 1983). Human IGF-II is encoded by the cDNA sequence published by Jensen M. et al. (FEBS 179:243-246, 1985). It will be understood that natural allelic variations exist and can occur among individuals, as demonstrated by one or more amino acid differences in the amino acid sequence of each individual.

The terms “IGF-I receptor” and “IGF-IR” when used herein refer to a cellular receptor for IGF-I and IGF-II, which typically includes an extracellular domain, a transmembrane domain and an intracellular domain, as well as variants and fragments thereof which retain the ability to bind IGF-I or IGF-II. The terms “IGF-I receptor” and “IGF-IR” encompasses soluble forms from natural sources, synthetically produced in vitro or obtained by genetic manipulation including methods of recombinant DNA technology. The IGF-IR variants or fragments preferably share at least about 65% sequence homology, and more preferably at least about 75% sequence homology with any domain of the human IGF-IR amino acid sequence published in Ullrich A. et al. (EMBO,. 5:2503-2512, 1986).

The term “IGF-I or IGF-II biological activity.” when used herein refers to any mitogenic, motogenic, anti-apoptotic or morphogenic activities of IGF-I or IGF-II or any activities occurring as a result of IGF-I or IGF-II binding to IGF-IR. The term “IGF-IR activation” refers to IGF-I or IGF-II-induced tyrosine kinase activity within the beta subunit of the IGF-IR. Activation of IGF-IR may occur as a result of IGF-I or IGF-II binding to IGF-IR, and although not described to date, may alternatively occur independent of IGF-I or IGF-II binding to the IGF-IR. In addition “IGF-IR activation” may occur following the binding of an IGF-IR monoclonal antibody to the IGF-IR. IGF-I or IGF-II biological activity may be determined, for example, in an in vitro or in vivo assay of IGF-I or IGF-II -induced cell proliferation, cell scattering, or cell migration. The effect of a IGF-IR receptor antagonist can be determined in an assay suitable for testing the ability of IGF-I or IGF-II to induce DNA synthesis in cells expressing IGF-IR such as mouse 3T3 human IGF-IR transfected fibroblast cells (described in Example 8). DNA synthesis can, for example, be assayed by measuring incorporation of ³ H-thymidine into DNA. The effectiveness of the IGF-IR antagonist can be determined by its ability to block proliferation and incorporation of the ³H-thymidine into DNA in response to IGF-I or IGF-II. The effect of IGF-IR antagonists can also-be tested in vivo in animal models.

The term “polypeptide” encompasses native or artificial proteins, protein fragments, and polypeptide analogs of a protein sequence. A polypeptide may be monomeric or polymeric.

The term “isolated protein” or “isolated polypeptide” is a protein or polypeptide that by virtue of its origin or source of derivation, (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein separation and purification techniques well known in the art.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally occurring sequence. Fragments typically are at least 5, 6, 8, or amino acids long, preferably at least 14 amino acids long, more preferably at least amino acids long, usually at least 20 amino acids long, even more preferably at least 70, 80, 90, 100, 150 or 200 amino acids long.

The term “polypeptide analog” as used herein refers to a polypeptide that is comprised of a segment of at least amino acids that has substantial identity to a portion of an amino acid sequence and that has at least one of the following properties: (1) specific binding to IGF-IR under suitable binding conditions, (2) ability to block IGF-I and IGF-II binding to IGF-IR, or (3) ability to reduce IGF-IR cell surface expression or tyrosine phosphorylation in vitro or in vivo. Typically, polypeptide analogs comprise a conservative amino acid substitution (or insertion or deletion) with respect to the naturally occurring sequence. Analogs typically are at least 20 amino acids long, preferably at least 50, 60, 70, 80, 90, 100, 150 or 200 amino acids long or longer, and can often be as long as a full-length naturally occurring polypeptide.

Preferred amino acid substitutions are those which, (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference. Non-peptide analogs are commonly used in the pharmaceutical industry as drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and-CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 1 or more amino acids primarily responsible for antigen recognition. The carboxy-terminal portion of each chain defines a constant region primarily responsible for effector function. Human light chains are classified as either kappa or lambda chains. Heavy chains are classified as μ, Δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarily determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat, et al., Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarily determining region (CDR) fragments, single-chain antibodies (scFv), chimeric antibodies, diabodies and polypeptides that contain at least a portion of an immunoglobulin that is sufficient to confer specific antigen binding to the polypeptide.

An Fab fragment is a monovalent fragment consisting of the VL, VH, CL and CH1 domains; a F(ab′)2 fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which a VL and VH regions are paired to form a monovalent molecule via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:64446448, 1993, and Poijak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally occurring immunoglobulin has two identical binding sites; a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature.

Examples of isolated antibodies include an IGF-IR antibody that has been affinity purified using IGF-IR as an antigen, an anti-IGF-IR antibody that has been synthesized by a hybridoma or other cell line in vitro, and a human IGF-IR antibody derived from a transgenic mouse.

The term “human antibody” includes all antibodies that have one or more variable and constant regions derived~ from human immunoglobulin sequences.

In a preferred embodiment, all of the variable and constant domains are derived from human immunoglobulin sequences (a fully human antibody). These antibodies may be prepared in a variety of ways, as described below.

A “humanized antibody” is an antibody that is derived from a non-human species, in which certain amino acids in the framework and constant domains of the. heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152, and 5,877,293.

The term “chimeric antibody” refers to an antibody that contains one or more regions from one antibody and one or more regions from one or more other antibodies. In a preferred embodiment, one or more of the CDRs are derived from a human IGF-IR antibody. In a more preferred embodiment, all of the CDRs are derived from a human IGF-IR antibody. In another preferred embodiment, the CDRs from more than one human IGF-IR antibody are mixed and matched in a chimeric antibody. For instance, a chimeric antibody may comprise-a CDR1 from the light chain of a first human IGF-IR antibody may be combined with CDR2 and CDR3 from the light chain of a second human IGF-IR antibody, and the CDRs from the heavy chain may be derived from a third IGF-IR antibody. Further, the framework regions may be derived from one of the same IGF-IR antibodies, from one or more different antibodies, such as a human antibody, or from a humanized antibody. A “neutralizing antibody” or “an inhibitory antibody” is an antibody that inhibits the binding of IGF-IR to IGF-I and IGF-II when an excess of the IGF-IR antibody reduces the amount of IGF-I and IGF-II bound to IGF-IR by at least about 20%. In a preferred embodiment, the antibody reduces the amount of IGF-I and IGF-II bound to IGF-IR by at least 40%, more preferably 60%, even more preferably 80%, or even more preferably 85%. The binding reduction may be measured by any means known to one of ordinary skill in the art, for example, as measured in an in vitro competitive binding assay. An example of measuring the reduction in binding of IGF-I and IGF-II to IGF-IR is presented below in Example 4.

An “activating antibody” is an antibody that activates IGF-IR by at least about 20% when added to a cell, tissue, or organism expressing IGF-IR, when compared to the activation achieved-by an equivalent molar amount of IGF-I and IGF-II. In a preferred embodiment, the antibody activates IGF-IR activity by at least 40%, more preferably 60%, even more preferably 80%, or even more preferably 85% of the level of activation achieved by an equivalent molar amount of IGF-I and IGF-II. In a more preferred embodiment, the activating antibody is added in the presence of IGF-I and IGF-II. In another preferred embodiment, the activity of the activating antibody is measured by determining the amount of tyrosine phosphorylation and activation of IGF-IR.

Fragments or analogs of antibodies can be readily prepared by those of ordinary skill in the art following the teachings of this specification. Preferred amino and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure have been described by Bowie et al. Science 253:164(1991).

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. Ann. Biol. Clin. 51:19-26 (1993); Jonsson, U., et al. Biotechniques 11:620-627 (1991); Johnsson, B., et al. J. Mol. Recognit. 8:125-131 (1995); and Johnsson, B., et al. Anal. Biochem. 198:268-277 (1991).

The term “K_{off}” refers to the off rate constant for dissociation of an antibody from the antibody/antigen complex.

The term “K_{d}” refers to the dissociation constant of a particular antibody-antigen interaction.

The term “epitope” includes any molecular determinant capable of specific binding to an immunoglobulin or T-cell receptor. Epitopes usually consist of chemically active surface groupings of molecules such as amino acids or sugar side chains and usually have specific three-dimensional structural characteristics, as well as specific charge characteristics. An antibody is said to specifically bind an antigen when the dissociation constant is <1 M, preferably <100 nM, preferably <10 nM, and most preferably <1 nM.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass.(1991)), which is incorporated herein by reference. Stereoisomers (e.g., D-amino acids) of the twenty conventional amino acids, unnatural amino acids such as α-, α-2,5 disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methyl arginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide”, (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotides” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or to 40 bases in length. Oligonucleotides are usually single stranded, e.g. for probes, although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like.

The term “oligonucleotide linkages” referred to herein includes Oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,51.0; Uhlmann and Peyman Chemical Reviews 90:543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes. generally, such control sequences include promoters and transcription termination sequence. The term “control sequences” is intended to include, at a minimum, all components whose presence is essential for expression and processing, and can also include additional components whose presence is advantageous, for example, leader sequences, and fusion partner sequences. The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked.

Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides, and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, SX Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art that can be used to measure nucleotide sequence identity. For instance, polynucleotide sequences can be compared using FASTA, Gap, or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998; herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleic acid sequence encompasses its complement unless otherwise specified. Thus, a reference to a nucleic acid molecule having a particular sequence should be understood to encompass its complementary strand, with its complementary sequence.

In the molecular biology art, researchers use the terms “percent sequence identity”, “percent sequence similarity” and “percent sequence homology” interchangeably. In this application, these terms shall have the same meaning with respect to nucleic acid sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, share at least 75% or 80% sequence identity, preferably at least 90% or 95% sequence identity, even more preferably at least 98% or 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e. g., charge or hydrophobicity). In general, a conservative amino, acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson, Methods Mol. Biol. 24: 307-31 (1994), herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions, and other modifications, including conservative amino acid substitutions. For instance, GCG contains programs such as “Gap” and “Bestfit” which can be used with default parameters to determine sequence homology or sequence identity between closely related polypeptides, such as homologous.

Polypeptides from different species of organisms or between a wild type protein and a mutein thereof. See, e.g.,.GCG Version 6.1. Polypeptide sequences also can be compared using FASTA using default or recommended parameters, a program in GCG Version 6.1. FASTA (e.g.,° FASTA2 and FASTA3) provides alignments and percent sequence identity of the regions of the best overlap between the query and search sequences (Pearson (1990); Pearson (2000). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215:403410 (1990); Altschul et al., Nucleic Acids Res. 25:3389-402 (1997); herein incorporated by reference.

The length of polypeptide sequences compared for homology will generally be at least about 16 amino acid residues, usually at least about residues, more usually at least about 24 residues, typically at least about 28 residues, and preferably more than about 35 residues. When searching a database containing sequences from a large number of different organisms, it is preferable to compare amino acid sequences.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B. gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

In some embodiments, labels are attached by spacer arms of various lengths to reduce potential steric hindrance.

The term “agent” is used herein to denote a chemical compound, a mixture of chemical compounds, a biological macromolecule, or an extract made from biological materials. The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

The term “patient” includes human and veterinary subjects.

Human IGF-IR Antibodies and Characterization Thereof

Human antibodies avoid certain of the problems associated with antibodies that possess mouse or rat variable and/or constant regions. The presence of such mouse or rat derived sequences can lead to the rapid clearance of the antibodies or can lead to the generation of an immune response against the antibody by a patient.

Therefore, in one embodiment, the invention provides humanized anti-IGF-IR antibodies. In a preferred embodiment, the invention provides fully human IGF-IR antibodies by introducing human immunoglobulin genes into a rodent so that the rodent produces fully human antibodies. More preferred are fully human anti-human IGF-IR antibodies. Fully human IGF-IR antibodies directed against human IGF-IR are expected to minimize the immunogenic and allergic responses intrinsic to mouse or mouse-derivatized monoclonal antibodies (Mabs) and thus to increase the efficacy and safety of the administered antibodies. The use of fully human antibodies can be expected to provide a substantial advantage in the treatment of chronic and recurring human diseases, such as inflammation and cancer, which may require repeated antibody administrations. In another embodiment, the invention provides an IGF-IR antibody that does not bind complement.

In a preferred embodiment, the IGF-IR antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 or a fragment of any one thereof. In a preferred embodiment, the IGF-IR antibody is selected from PINT-7A4, PINT-8A1, PINT-9A2, PINT-11A1, and PINT-11A4 or a fragment of any one thereof. In a preferred embodiment the IGF-IR antibody is selected from PINT-8A1, PINT-9A2, and PINT-11A4 or a fragment of any one thereof.

Table 1 shows the amino acid sequences of the scFvs PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 antibodies above.

TABLE 1

| | |
|---|---|
| PINT 6A1 | |
| EVQLVQSGAEVKKPGESLTISCKGSGYNFFNYWIGWVRQMPGKGLEWMGIIYPTDSD | SEQ ID NO:1 |
| TRYSPSFQGQVTISVDKSISTAYLQWSSLKASDTAMYYCARSIRYCPGGRCYSGYYG | |
| MDVWGRGTMVTVSSGGGGSGGGGSGGGGSSELTQDPAVSVALGQTVRITCQGDSLRS | |
| YYASWYQQKPGQAPVLVIYGKNKRPSGIPDRFSGSSSGNTASLTITGAQAEDEADYY | |
| CHSRDSSGNHVLFGGGTKLTVLG, | |
| PINT 7A2 | |
| GVQLVQSGAEVKKPGESLTISCKGSGYNFFNYWIGWVRQMPGKGLEWMGIIYPTDSD | SEQ ID NO:2 |
| TRYSPSFQGQVTISVDKSISTAYLQWSSLKASDTAMYYCARSIRYCPGGRCYSGYYG | |
| MDVWGQGTMVTVSSGGGGSGGGGSGGGGSSELTQDPAVSVALGQTVRITCQGDSLRS | |
| YYTNWFQQKPGQAPLLVVYAKNKRPSGIPDRFSGSSSGNTASLTITGAQAEDEADYY | |
| CNSRDSSGNHVVFGGGTKLTVLG, | |
| PINT 7A4 | |
| EVQLVQSGAEVKKPGESLTISCKGSGYNFFNYWIGWVRQMPGKDLEWMGIIYPTDSD | SEQ ID NO:3 |
| TRYSPSFQGQVTISVDKSISTAYLQWSSLKASDTAMYYCARSIRYCPGGRCYSGYYG | |
| MDVWGQGTMVTVSSGGGSSGGGGSGGGGSSELTQDPAVSVALGQTVRITCRGDSLRN | |
| YYASWYQQKPGQAPVLVIYGKNNRPSGIPDRFSGSSSGNTASLTITGAQAEDEADYY | |
| CNSRDSSGNHMVFGGGTKLTVLG, | |
| PINT 7A5 | |
| GVQLVESGAEVKKPGESLTISCKGSGYNFFNYWIGWVRQMPGKGLEWMGIIYPTDSD | SEQ ID NO:4 |
| TRYSPSFQGQVTISVDKSISTAYLQWSSLKASDTAMYYCARSIRYCPGGRCYSGYYG | |
| MDVWGRGTLVTVSSGGGGSGGGGSGGGGSSELTQDPAVSVALGQTVRITCQGDSLRS | |
| YYASWYQQKPGQAPVLVIYGKNNRPSGIPDRFSGSSSGNTASLTITGAQAEDEADYY | |
| CNSRDSSGNHVVFGGGTKLTVLG, | |
| PINT 7A6 | |
| EVQLVQSGAEVKKPGESLTISCKGSGYNFFNYWIGWVRQMPGKGLEWMGIIYPTDSD | SEQ ID NO:5 |
| TRYSPSFQGQVTISVDKSISTAYLQWSSLKASDTAMYYCARSIRYCPGGRCYSGYYG | |
| MDVWGQGTLVTVSSGGGGSGGGGSGGGGSSELTQDPAVSVALGQTVRITCQGDSLRS | |
| YYTNWFQQKPGQAPLLVVYAKNKRPSGIPDRFSGSSSGNTASLTITGAQAEDEADYY | |
| CNSRDSSGNHVVFGGGTKLTVLG, | |
| PINT 8A1 | |
| EVQLVQSGAEVKKPGESLTISCKGPGYNFFNYWIGWVRQMPGKGLEWMGIIYPTDSD | SEQ ID NO:6 |
| TRYSPSFQGQVTISVDKSISTAYLQWSSLKASDTAMYYCARSIRYCPGGRCYSGYYG | |
| MDVWGQGTMVTVSSGGGGSGGGGSGGGGSSELTQDPAVSVALGQTVRITCQGDSLRS | |
| YYASWYQQKPGQAPVLVIYGKNNRPSGIPDRFSGSSSGNTASLTITGAQAEDEADYY | |
| CNSRDSSGNHVVFGGGTKLTVLG, | |
| PINT 9A2 | |
| QVQLVQSGAEVRKPGASVKVSCKTSGYTFRNYDINWVRQAPGQGLEWMGRISGHYGN | SEQ ID NO:7 |
| TDHAQKFQGRFTMTKDTSTSTAYMELRSLTFDDTAVYYCARSQWNVDYWGRGTLVTV | |
| SSGGGGSGGGGSGGGGSALNFMLTQPHSVSESPGKTVTISCTRSSGSIASNYVQWYQ | |
| QRPGSSPTTVIFEDNRRPSGVPDRFSGSIDTSSNSASLTISGLKTEDEADYYCQSFD | |
| STNLVVFGGGTKVTVLG, | |
| PINT 11A1 | |
| EVQLVESGGGVVQPGRSLRLSCAASGFTFSDFAMHWVRQIPGKGLEWLSGLRHDGST | SEQ ID NO:8 |
| AYYAGSVKGRFTISRDNSRNTVYLQMNSLRAEDTATYYCVTGSGSSGPHAFPVWGKG | |
| TLVTVSSGGGGSGGGGSGGGGSALSYVLTQPPSASGTPGQRVTISCSGSNSNIGTYT | |
| VNWFQQLPGTAPKLLIYSNNQRPSGVPDRFSGSKSGTSASLAISGLQSEDEADYYCA | |
| A WDDSLNGPVFGGGTKVTVLG, | |
| PINT 11A2 | |
| EVQLLESGGGLVQPGGSLRLSCAASGFTFSSYAMSWVRQAPGKGLEWVSAISGSGGS | SEQ ID NO: 9 |
| TYYADSVKGRFTISRDNSKNTLYLQMNSLRAEDTAVYYCAKGMGYYGSGGYYPDDAF | |
| DVWGQGTMVTVSSGGGGSGGGGSGGGGSALSSELTQDPDVSMALGQTVTISCRGDSL | |
| KRFYASWYHQKPGQAPVLVFYGKENRPSGIPDRFSGSDSGDTASLTITGAQAEDEGD | |
| YYCHTQDTSARQYVFGSGTKVTVLG, | |
| PINT 11A3 | |
| EVQLVQSGAEVKKPGASVKVSCKASGYSFTNYGLNWVRQAPGQGLEWMGWISPYTGY | SEQ ID NO:10 |
| TNYAQKFQGRVTMTTDKSTSTAYMDLRSLRSDDTAVYYCAREIFSHCTGGSCYPFDS | |
| WGRGTLVTVSSGGGGSGGGGSGGGGSALSSELTQDPAVSVALGQTVRITCQGDSLRN | |
| YYASWYQQKPGQAPLLVMFGKNNRPSEIPGRFSGSSSGNTASLTITGAQAEDEADYY | |
| CNSRDRNSHQWVFGGGTKLTVLG, | |
| PINT 11A4 | |
| EVQLLESGGGLVQPGGSLRLSCAASGFTFSSYAMSWVRQAPGKGLEWVSAISGSGGS | SEQ ID NO:11 |
| TYYADSVKGRFTISRDNSKNTLYLQMNSLRAEDTAVYYCASSPYSSRWYSFDPWGQG | |
| TMVTVSSGGGGSGGGGSGGGGSALSYELTQPPSVSVSPGQTATITCSGDDLGNKYVS | |
| WYQQKPGQSPVLVIYQDTKRPSGIPERFSGSNSGNIATLTISGTQAVDEADYYCQVW | |
| DTGTVVFGGGTKLTVLG, | |
| PINT 11A5 | |
| QVQLVQSGAEVKKPGASVKVSCKASGYSFTNYGLNWVRQAPGQGLEWMGWISPYTGY | SEQ ID NO:12 |
| TNYAQKFQGRVTMTTDKSTSTAYMDLRSLRSDDTAVYYCAREIFSHCTGGSCYPFDS | |
| WGKGTLVTVSSGGGGSGGGGSGGGGSALSSELTQDPAVSVALGQTVRITCQGDSLRS | |
| YYASWYQQKPGQAPVLVIYGKNNRPSGIPDRFSGSSSGNTASLTITGAQAEDEADYY | |
| CNSRDSSGNHHWVFGGGTKVTVLG, | |
| PINT 11A7 | |
| EVQLVQSGAEVKKPGASVKVSCKASGYSFTNYGLDWVRQAPGQGLEWMGWISPYTGY | SEQ ID NO:13 |
| TNYAQKFQGRVTMTTDKSTSTAYMDLRSLRSDDTAVYYCAREIFSHCTGGSCYPFDS | |
| WGRGTMVTVSSGGGGSGGGGSGGGGSALSSELTQDPAVSVALGQTVRITCQGDSLRS | |
| YYASWYQQKPGQAPVLVIYGKNNRPSGIPDRFSGSSSGNTASLTITGAQAEDEADYY | |
| CNSRDSSGNHRNWVFGGGTKVTVLG, | |
| PINT 11A11 | |
| QVQLVESGGGLVKPGGSLRLSCAASGFTFSSHTMNWVRQAQGKGLEWVSSISGSGRY | SEQ ID NO:14 |
| IYYSDSVKGRFTISRDAAKNSLYLQMNNLRAEDTAVYYCTRAKFGDYLFDSWGQGTL | |
| VTVSSGGGGSGGGGSGGGGSALNFMLTQPHSVSQSPGKTVTISCTRSSGRIASNFVQ | |
| WYQQRPGSAPTTVIYEDNRRPSGVPDRFSGSIDSSSNSASLTISGLKTEDEADYYCQ | |
| SYDARYQVFGTGTKVTVLG, | |
| PINT 11A12 | |
| EVQLLESGGGLVQPGGSLRLSCAASGFTFSSYAMSWVRQAPGKGLEWVSAISGSGGS | SEQ ID NO:15 |
| TYYADSVKGRFTISRDNSKNTLYLQMNSLRAEDTAVYYCARSPVPPWADWYYFDYWG | |
| RGTMVTVSSGGGGSGGGGSGGGGSAQAVLTQPSSVSGAPGQRVTISCTGSRSNFGAG | |
| YDVHWYQQFPGTAPKLLIYGNTNRPSGVPDRFSGSRSGTSASLAITGLQAEDEADYY | |
| CQSYDSNLSGSVFGGGTKVTVLG, | |
| PINT 11A3 | |
| EVQLVQSGAEVKKPGASVKVSCKASGYSFTNYGLNWVRQAPGQGLEWMGWISPYTGY | SEQ ID NO:16 |
| TNYAQKFQGRVTMTTDKSTSTAYMDLRSLRSDDTAVYYCAREIFSHCTGGSCYPFDS | |
| WGKGTLVTVSSGGGGSGGGGSGGGGSALSSELTQDPAVSVALGQTVRITCQGDSLRN | |
| YYASWYQQKPGQAPVLVLYSKNSRPSGVPDRFSGSSSGTTASLTISGAQAEDEADYY | |
| CNSRDTSGDLRWVFGGGTKLTVLG, | |
| PINT 12A2 | |
| EVQLVQSGAEVKKPGASVKVSCKASGYSFTNYGLNWVRQAPGQGLEWMGWISPYTGY | SEQ ID NO:17 |
| TNYAQKFQGRVTMTTDKSTSTAYMDLRSLRSDDTAVYYCAREIFSHCTGGSCYPFDS | |
| WGQGTLVTVSSGGGGSGGGGSGGGGSALSSELTQDPAVSVALGQTVRITCQGDSLRN | |
| YYASWYQQKPGQAPLLVMFGKNNRPSEIPGRFSGSSSGNTASLTITGAQAEDEADYY | |
| CNSRDSNSHQWVFGGGTKLTVLG, | |
| PINT 12A3 | |
| QVQLVQSGAEVKKPGASVKVSCKASGYSFTNYGLNWVRQAPGQGLEWMGWISPYTGY | SEQ ID NO:18 |
| TNYAQKFQGRVTMTSDKSTSTAYMDLRSLRSDDTAIYYCAREIFSHCSGGSCYPFDY | |
| WGQGTLVTVSSGGGGSGGGGSGGGGSALSSELTQDPAVSVALGQTVRITCQGDSLRS | |
| YYASWYQQKPGQAPLLVIYGRNNRPSGIPDRFSGSSSGNTASLTITGAQAEDEADYY | |
| CNSRDSSTNHGNWVFGGGTQLTVLS, | |
| and | |
| PINT 12A4 | |
| QVQLVQSGAEVKKPGASVKVSCKASGYSFTNYGLNWVRQAPGQGLEWMGWISPYTGY | SEQ ID NO:19 |
| TNYAQKFQGRVTMTTDKSTSTAYMDLRSLRSDDTAVYYCAREIFSHCTGGSCYPFDS | |
| WGRGTMVTVSSGGGGSGGGGSGGGGSALSSELTQDPAVSVALGQTVRITCQGDSLRS | |
| YYASWYQQKPGQAPVLVIYGKNNRPSGIPDRFSGSSSGNTASLTITGAQAEDEADYY | |
| CNSRDSSGNLNWVFGGGTQLTVLS. | |

In another preferred embodiment, the IGF-IR antibody comprises a light chain amino acid sequence from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or one or more CDRs from these amino acid sequences. In another preferred embodiment, the IGF-IR antibody comprises a heavy chain amino acid sequence from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19 or one or more CDRs from these amino acid sequences.

Class and Subclass of IGF-IR Antibodies

The antibody may be an IgG, an IgM, an IgE, an IgA, or an IgD molecule. In a preferred embodiment, the antibody is an IgG and is an IgG1, IgG2, IgG3, or IgG4 subtype. In a more preferred embodiment, the IGF-IR antibody is subclass IgG1. In another preferred embodiment, the IGF-IR antibody is the same class and subclass as antibody PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5, which is IgG1.

The class and subclass of IGF-IR antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot, as well as other techniques.

Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

Molecule Selectivity

In another embodiment, the IGF-IR antibody has a selectivity for IGF-IR that is at least 50 times greater than its selectivity for insulin, Ron, Axl, NGF, and Mer receptors. In a preferred embodiment, the selectivity of the IGF-IR antibody is more than 100 times greater than for insulin, Ron, Axl, NGF, and Mer receptor. In an even more preferred embodiment, the IGF-IR antibody does not exhibit any appreciable specific binding to insulin. In an even more preferred embodiment, the IGF-IR antibody does not exhibit any appreciable specific binding to any other protein than IGF-IR. One may determine the selectivity of the IGF-IR antibody for IGF-IR using methods well known in the art following the teachings of the specification. For instance, one may determine the selectivity using Western blot, FACS, ELISA, or RIA. In a preferred embodiment, one may determine the molecular selectivity using Western blot.

Binding Affinity of IGF-IR antibody to IGF-IR

In another aspect of the invention, the IGF-IR antibodies bind to IGF-IR with high affinity. In one embodiment, the IGF-IR antibody binds to IGF-IR with a K_{d} of 1×10⁻⁸ M or less. In a more preferred embodiment, the antibody binds to IGF-IR with a K_{d} or 1×10⁻⁹ M or less. In an even more preferred embodiment, the antibody binds to IGF-IR with a K_{d} or 5×10⁻¹⁰ M or less. In another preferred embodiment, the antibody binds to IGF-IR with a K_{d} of 1×10⁻¹⁰ M or less. In another preferred embodiment, the antibody binds to IGF-IR with substantially the same K_{d} as an antibody selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In another preferred embodiment, the antibody binds to IGF-IR with substantially the same K_{d} as an antibody that comprises one or more CDRs from an antibody selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In still another preferred embodiment, the antibody binds to IGF-IR with substantially the same K_{d} as an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In another preferred embodiment, the antibody binds to IGF-IR with substantially the same K_{d} as an antibody that comprises one or more CDRs from an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

In another aspect of the invention, the IGF-IR antibody has a low dissociation rate. In one embodiment, the IGF-IR antibody has a K_{off} of 1×10⁻¹ s⁻¹ or lower. In a preferred embodiment, the K_{off} is 5×10⁻⁵ s⁻¹ or lower. In another preferred embodiment, the K_{off} is substantially the same as an antibody selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In another preferred embodiment, the antibody binds to IGF-IR with substantially the same K_{off} as an antibody that comprises one or more CDRs from an antibody selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In still another preferred embodiment, the antibody binds to IGF-IR with substantially the same K_{off} as an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In another preferred embodiment, the antibody binds to IGF-IR with substantially the same K_{off} as an antibody that comprises one or more CDRs from an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or a fragment thereof.

The binding affinity and dissociation rate of an IGF-IR antibody to IGF-IR may be determined by any method known in the art. In one embodiment, the binding affinity can be measured by competitive ELISAs, RIAs, or surface plasmon resonance, such as BIAcore. The dissociation rate can also be measured by surface plasmon resonance. In a more preferred embodiment, the binding affinity and dissociation rate is measured by surface plasmon resonance. In an even more preferred embodiment, the binding affinity and dissociation rate is measured using a BIAcore. An example of determining binding affinity and dissociation rate for binding of IGF-IR antibodies to the extracellular domain of human IGF-IR using BIAcore is described below in Example 10.

Half-Life IGF-IR Antibodies

According to another object of the invention, the IGF-IR antibody has a half-life at least one day in vitro or in vivo. In a preferred embodiment, the antibody or portion thereof has a half-life of at least three days. In a more preferred embodiment, the antibody or portion thereof has a half-life of four days or longer. In another embodiment, the antibody or portion thereof has a half-life of eight days or longer. In another embodiment, the antibody or antigen-binding portion thereof is derivatized or modified such that it has a longer half-life, as discussed below.

In another preferred embodiment, the antibody may contain point mutations to increase serum half-life, such as described WO 00/09560, published Feb. 24, 2000.

The antibody half-life may be measured by any means known to one having ordinary skill in the art. For instance, the antibody half-life may be measured by Western blot, ELISA or RIA over an appropriate period of time. The antibody half-life may be measured in any appropriate animals, e.g., a monkey, such as a cynomolgus monkey, a primate or a human.

The invention also provides an IGF-IR antibody that binds the same antigen or epitope as a human IGF-IR antibody of the present invention. Further, the invention provides an IGF-IR antibody that cross-competes with an IGF-IR antibody known to block IGF-I and IGF-II binding. In a highly preferred embodiment, the known IGF-IR antibody is another human antibody. In a preferred embodiment, the human IGF-IR antibody has the same antigen or epitope of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the human IGF-IR antibody comprises one or more CDRs from an antibody that binds the same antigen or epitope of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In still another preferred embodiment, the human IGF-IR antibody that binds the same antigen or epitope comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or a fragment thereof. In another preferred embodiment, the human IGF-IR antibody that binds the same antigen or epitope comprises one or more CDRs from an antibody of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

One may determine whether an IGF-IR antibody binds to the same antigen using a variety of methods known in the art. For instance, one may determine whether a test IGF-IR antibody binds to the same antigen by using a IGF-IR antibody to capture an antigen that is known to bind to the IGF-IR antibody, such as IGF-IR, eluting the antigen from the antibody, and determining whether the test antibody will bind to the eluted antigen. One may determine whether the antibody binds to the same epitope as an IGF-IR antibody by binding the IGF-IR antibody to IGF-IR under saturating conditions, and then measuring the ability of the test antibody to bind to IGF-IR. If the test antibody is able to bind to the IGF-IR at the same time as the IGF-IR antibody, then the test antibody binds to a distinct epitope from the IGF-IR antibody. However, if the test antibody is not able to bind to the IGF-IR at the same time, then the test antibody binds to the same epitope, or shares an overlapping epitope binding site, as the human IGF-IR antibody. This experiment may be performed using ELISA, RIA, or surface plasmon resonance. In a preferred embodiment, the experiment is performed using surface plasmon resonance. In a more preferred embodiment, BIAcore is used. One may also determine whether an IGF-IR antibody cross-competes with another IGF-IR antibody. In a preferred embodiment, one may determine whether an IGF-IR antibody cross-competes with another by using the same method that is used to measure whether the IGF-IR antibody is able to bind to the same epitope as another IGF-IR antibody.

Light and Heavy Chain Usage

The invention also provides an IGF-IR antibody that comprises variable sequences encoded by a human λ (Williams S. C. et al., J. Mol. Biol. 264:220-232, 1996) or κ gene (Kawvasaki K. et al., Eur. J. Immunol. 31:1017-1028, 2001). In a preferred embodiment, the light chain variable sequences are encoded by the Vλ 1e, 1c, 3r, 3i, or 6a gene family. In one embodiment, the variable sequences are encoded by the Vκ A27, A30, or O12 gene family. In a more preferred embodiment, the light chain comprises no more than ten amino acid substitutions from the germline, preferably no more than six amino acid substitutions, and more preferably no more than three amino acid substitutions. In a preferred embodiment, the amino acid substitutions are conservative substitutions.

SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, provide the amino acid sequences of the variable regions of IGF-IR antibody λ light chains. Following the teachings of this specification, one of ordinary skill in the art could determine the encoded amino acid sequence of the IGF-IR antibody light chains and the germline light chains and determine the differences between the germline sequences and the antibody sequences.

In a preferred embodiment, the VL of the IGF-IR antibody contains the same amino acid substitutions, relative to the germline amino acid sequence, as any one or more of the VL of antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. For example, the VL of the IGF-IR antibody may contain one or more amino acid substitutions that are the same as those present in antibody PGIA-03-A9, another amino acid substitution that is the same as that present in antibody PGIA-03-B2, and another amino acid substitution that is the same as antibody PGIA-01-A8. In this manner, one can mix and match different features of antibody binding in order to alter, e.g., the affinity of the antibody for IGF-IR or its dissociation rate from the antigen. In another embodiment, the amino acid substitutions are made in the same position as those found in any one or more of the VL of antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5, but conservative amino acid substitutions are made rather than using the same amino acid. For example, if the amino acid substitution compared to the germline in one of the antibodies PINT-6A1, PINT-7A2, PINT-7A4, PINT-7A5, PINT-7A6, PINT-8A1, PINT-9A2,. PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5 is glutamate, one may conservatively substitute aspartate.

Similarly, if the amino acid substitution is serine, one may conservatively substitute threonine. In another preferred embodiment, the light chain comprises an amino acid sequence that is the same as the amino acid sequence of the VL of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A 12, PINT-12A1, PINT-12A2, PINT-12A3, PINT-12A4, or PINT-12A5. In another highly preferred embodiment, the light chain comprises amino acid sequences that are the same as the CDR regions of the light chain of PINT-6A1, PINT-7A2, PINT-7A4, PINT-7A5, PINT-7A6, PINT-8A], PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the light chain comprises an amino acid sequence from at least one CDR region of the light chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the light chain comprises amino acid sequences from CDRs from different light chains. In a more preferred embodiment, the CDRs from different light chains are obtained from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the light chain comprises a VL amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In another embodiment, the light chain comprises an amino acid sequence encoded by a nucleic acid sequence selected from SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38, fragments thereof, or a nucleic acid sequence that encodes an amino acid sequence having 1-10 amino acid insertions, deletions or substitutions therefrom. Preferably, the amino acid substitutions are conservative amino acid substitutions. In another embodiment, the antibody or portion thereof comprises a lambda light chain.

The present invention also provides an IGF-IR antibody or portion thereof, which comprises a human heavy chain or a sequence derived from a human heavy chain. In one embodiment, the heavy chain amino acid sequence is derived from a human VH DP-14, DP-47, DP-50, DP-73, or DP-77 gene family. In a more preferred embodiment, the heavy chain comprises no more than eight amino acid changes from germline, more preferably no more than six amino acid changes, and even more preferably no more than three amino acid changes.

SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, provide the amino acid sequences of the variable regions of IGF-IR antibody heavy chains. Following the teachings of this specification, one of ordinary skill in the art could determine the encoded amino acid sequence of the IGF-IR antibody heavy chains and the germline heavy chains and determine the differences between the germline sequences and the antibody sequences.

In a preferred embodiment, the VH of the IGF-IR antibody contains the same amino acid substitutions, relative to the germline amino acid sequence, as any one or more of the VH of antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. Similar to what was discussed above, the VH of the IGF-IR antibody may contain one or more amino acid substitutions that are the same as those present in antibody PINT-8A1, another amino acid substitution that is the same as that present in antibody PINT-9A2, and another amino acid substitution that is the same as antibody PINT-11A4. In this manner, one can mix and match different features of antibody binding in order to alter, e.g., the affinity of the antibody for IGF-IR or its dissociation rate from the antigen. In another embodiment, the amino acid substitutions are made in the same position as those found in any one or more of the VH of antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, but conservative amino acid substitutions are made rather than using the same amino acid.

In another preferred embodiment, the heavy chain comprises an amino acid sequence that is the same as the amino acid sequence of the VH of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another highly preferred embodiment, the heavy chain comprises amino acid sequences that are the same as the CDR regions of the heavy chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the heavy chain comprises an amino acid sequence from at least one CDR region of the heavy chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5A1. In another preferred embodiment, the heavy chain comprises amino acid sequences from CDRs from different heavy chains. In a more preferred embodiment, the CDRs from different heavy chains are obtained from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another preferred embodiment, the heavy chain comprises a VH amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. In another embodiment, the heavy chain comprises a VH amino acid sequence encoded by a nucleic acid sequence selected from SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38, a fragment thereof, or a nucleic acid sequence that encodes an amino acid sequence having 1-10 amino acid insertions, deletions or substitutions therefrom. In another embodiment, the substitutions are conservative amino acid substitutions.

Table 2 shows a nucleic acid sequences encoding the scFvs PGIA-01-A1 through PGIA-05-A1.

TABLE 2

| | |
|---|---|
| PINT 6A1 | |
| GAAGTGCAGCTGGTGCAGTCTGGAGCAGAGGTGAAAAAGCCCGGGGAGTCTCTGACA | SEQ ID NO:20 |
| ATCTCCTGTAAGGGTTCTGGGTACAACTTTTTCAACTACTGGATCGGCTGGGTGCGC | |
| CAGATGCCCGGGAAAGGCCTGGAGTGGATGGGGATCATCTATCCTACTGACTCTGAT | |
| ACCAGATATAGCCCGTCCTTCCAAGGCCAGGTCACCATTTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCGGGGGACAATGGTCACCGTCTCTTCAGGTGGAGGCGGTTCA | |
| GGCGGAGGTGGCAGCGGCGGTGGCGGATCGTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAATAAGCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTCATTCCCGGGACAGCAGTGGTAACCATGTGCTTTTCGGCGGAGGGACCAAGCTG | |
| ACCGTCCTAGGT, | |
| PINT 7A2 | |
| GGGGTGCAGCTGGTGCAGTCTGGGGCAGAGGTGAAAAAGCCCGGGGAGTCTCTGACA | SEQ ID NO:21 |
| ATCTCCTGTAAGGGTTCTGGATACAACTTTTTCAACTACTGGATCGGCTGGGTGCGC | |
| CAGATGCCCGGGAAAGGCCTGGAGTGGATGGGGATCATCTATCCTACTGACTCTGAT | |
| ACCAGATATAGCCCGTCCTTCCAAGGCTAGGTCACCATCTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCAGGGGACAATGGTCACCGTCTCGAGTGGTGGAGGCGGTTCA | |
| GGCGGAGGTGGCAGCGGCGGTGGCGGATCGTCTGAGTTGACTCAGGACCCTGCTGTG | |
| TCTGTGGCCTTGGGACAGACAGTCAGGATCACTTGCCAAGGAGACAGTCTCAGAAGC | |
| TATTACACAAACTGGTTCCAGCAGAAGCCAGGACAGGCCCCTCTACTTGTCGTCTAT | |
| GCTAAAAATAAGCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATGTGGTATTCGGCGGAGGGACCAAGCTG | |
| ACCGTCCTAGGT, | |
| PINT 7A4 | |
| GAAGTGCAGCTGGTGCAGTCTGGGGCAGAGGTGAAAAAGCCCGGGGAGTCTCTGACA | SEQ ID NO:22 |
| ATCTCCTGCAAGGGTTCTGGATACAACTTTTTCAACTACTGGATCGGCTGGGTGCGC | |
| CAGATGCCCGGGAAAGACCTGGAGTGGATGGGGATCATCTATCCTACTGACTCTGAT | |
| ACCAGATATAGCCCGTCCTTCCAAGGCCAGGTCACGATTTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCAGGGGACAATGGTCACCGTCTCGAGTGGTGGAGGCAGTTCA | |
| GGCGGAGGTGGCAGCGGCGGTGGCGGATCGTCTGAGCTGACTCAGGACCCTGCTGTG | |
| TCTGTGGCCTTGGGACAGACAGTCAGGATCACATGCCGAGGAGACAGCCTCAGAAAC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATATGGTATTCGGCGGAGGGACCAAGCTG | |
| ACCGTCCTAGGT, | |
| PINT 7A5 | |
| GGGGTGCAGCTGGTGGAGTCTGGGGCAGAGGTGAAAAAGCCCGGGGAGTCTCTGACA | SEQ ID NO:23 |
| ATCTCCTGTAAGGGTTCTGGATACAACTTTTTCAACTACTGGATCGGCTGGGTGCGC | |
| CAGATGCCCGGGAAAGGCCTGGAGTGGATGGGGATCATCTATCCTACTGACTCTGAT | |
| ACCAGATATAGCCCGTCCTTCCAAGGCCAGGTCACCATCTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCGGGGAACCCTGGTCACCGTCTCCTCAGGTGGAGGCGGTTCA | |
| GGCGGAGGTGGCAGCGGCGGTGGCGGATCGTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATGTGGTATTCGGCGGAGGGACCAAGCTG | |
| ACCGTCCTAGGT, | |
| PINT 7A6 | |
| GAAGTGCAGCTGGTGCAGTCTGGAGCAGAGGTGAAAAAGCCCGGGGAGTCTCTGACA | SEQ ID NO:24 |
| ATCTCCTGTAAGGGTTCTGGATACAACTTTTTCAACTACTGGATCGGCTGGGTGCGC | |
| CAGATGCCCGGGAAAGGCCTGGAGTGGATGGGGATCATCTATCCTACTGACTCTGAT | |
| ACCAGATATAGCCCGTCCTTCCAAGGCCAGGTCACCATTTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCAGGGCACCCTGGTCACCGTCTCCTCAGGTGGAGGCGGTTCA | |
| GGCGGAGGTGGCAGCGGCGGTGGCGGATCGTCTGAGCTGACTCAGGACCCTGCTGTG | |
| TCTGTGGCCTTGGGACAGACAGTCAGGATCACTTGCCAAGGAGACAGTCTCAGAAGC | |
| TATTACACAAACTGGTTCCAGCAGAAGCCAGGACAGGCCCCTCTACTTGTCGTCTAT | |
| GCTAAAAATAAGCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATGTGGTATTCGGCGGAGGGACCAAGCTG | |
| ACCGTCCTAGGT, | |
| PINT 8A1 | |
| GAGGTGCAGCTGGTGCAGTCTGGGGCAGAGGTGAAAAAGCCCGGGGAGTCTCTGACA | SEQ ID NO:25 |
| ATCTCCTGTAAGGGTCCTGGATACAACTTTTTCAACTACTGGATCGGCTGGGTGCGC | |
| CAGATGCCCGGGAAAGGCCTGGAGTGGATGGGGATCATCTATCCTACTGACTCTGAT | |
| ACCAGATATAGCCCGTCCTTCCAAGGCCAGGTCACCATCTCAGTCGACAAGTCCATT | |
| AGCACCGCCTATCTGCAGTGGAGCAGCCTGAAGGCCTCCGACACCGCCATGTATTAC | |
| TGTGCGAGATCCATTAGATACTGTCCTGGTGGTAGGTGCTACTCCGGTTACTACGGT | |
| ATGGACGTCTGGGGCCAAGGAACCATGGTCACCGTCTCCTCAGGTGGAGGCGGTTCA | |
| GGCGGAGGTGGCAGCGGCGGTGGCGGATCGTCTGAGCTGACTCAGGACCCTGCTGTG | |
| TCTGTGGCCTTGGGACAGACGGTCAGCATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATGTGGTATTCGGCGGAGGGACCAAGCTG | |
| ACCGTCCTAGGT, | |
| PINT 9A2 | |
| CAGGTCCAGCTGGTGCAGTCTGGGGCTGAAGTGAGGAAGCCTGGGGCCTCAGTGAAG | SEQ ID NO:26 |
| GTCTCCTGCAAGACTTCAGGTTACACCTTTAGGAACTATGATATCAACTGGGTGCGA | |
| CAGGCCCCTGGACAAGGGCTTGAGTGGATGGGAAGGATCAGTGGTCACTATGGCAAC | |
| ACAGACCATGCACAGAAATTCCAGGGCAGATTCACCATGACCAAAGACACATCCACG | |
| AGCACAGCCTACATGGAACTGAGGAGCCTGACATTTGACGACACGGCCGTATATTAC | |
| TGTGCGAGAAGTCAGTGGAACGTTGACTACTGGGGCCGAGGAACCCTGGTCACCGTC | |
| TCGAGTGGAGGCGGCGGTTCAGGCGGAGGTGGCTCTGGCGGTGGCGGAAGTGCACTT | |
| AATTTTATGCTGACTCAGCCCCACTCTGTGTCGGAGTCTCCGGGGAAGACGGTGACC | |
| ATCTCCTGCACCCGCAGCAGTGGCAGCATTGCTAGCAATTATGTGCAGTGGTACCAG | |
| CAGCGCCCGGGCAGTTCCCCCACCACTGTGATCTTTGAAGATAACCGAAGACCCTCT | |
| GGGGTCCCTGATCGGTTTTCTGGCTCCATCGACACCTCCTCCAACTCTGCCTCCCTC | |
| ACCATCTCTGGACTGAAGACTGAGGACGAGGCTGACTACTACTGTCAGTCTTTTGAT | |
| AGCACCAATCTTGTGGTGTTCGGCGGAGGGACCAAGGTCACCGTCCTAGGT, | |
| PINT 11A1 | |
| GAGGTGCAGCTGGTGGAGTCTGGGGGAGGCGTGGTCCAGCCTGGGAGGTCCCTGAGA | SEQ ID NO:27 |
| CTCTCCTGTGCAGCGTCTGGCTTCACTTTCAGTGATTTTGCCATGCACTGGGTCCGC | |
| CAGATTCCAGGCAAGGGGCTGGAGTGGCTGTCAGGATTACGGCATGATGGAAGTACG | |
| GCTTACTATGCAGGGTCCGTGAAGGGCCGCTTCACCATCTCCAGAGACAATTCCAGG | |
| AATACTGTATATCTCCAAATGAATAGCCTGAGGGCCGAGGACACGGCTACGTATTAC | |
| TGTGTGACAGGGAGCGGTAGCTCCGGTCCCCACGCTTTTCCTGTCTGGGGCAAAGGC | |
| ACCCTGGTCACCGTCTCGAGTGGAGGCGGCGGTTCAGGCGGAGGTGGCTCTGGCGGT | |
| GGCGGAAGTGCACTTTCCTATGTGCTGACTCAGCCACCCTCAGCGTCTGGGACCCCC | |
| GGGCAGAGGGTCACCATCTCTTGTTCTGGAAGCAACTCCAACATCGGGACTTATACT | |
| GTAAATTGGTTCCAGCAGCTCCCAGGAACGGCCCCCAAACTCCTCATCTACAGTAAT | |
| AATCAGCGGCCCTCAGGGGTCCCTGACCGATTCTCTGGCTCCAAGTCTGGCACCTCA | |
| GCCTCCCTGGCCATCAGTGGGCTCCAGTCTGAGGATGAGGCTGATTATTACTGTGCA | |
| GCAATGGGATGACAGCCTGAATGGTCCGGTTTTCGGCGGAGGGACCAAGGTCACCGT | |
| CCTAGGTGCGGCCGCACATCATCATCACCATCA, | |
| PINT 11A2 | |
| GAGGTGCAGCTGTTGGAGTCTGGGGGAGGCTTGGTACAGCCTGGGGGGTCCCTGAGA | SEQ ID NO:28 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAAAGGAATGGGATACTATGGTTCGGGAGGTTATTATCCGGATGATGCTTTT | |
| GATGTCTGGGGCCAGGGGACAATGGTCACCGTCTCGAGTGGAGGCGGCGgTTCAGGC | |
| GGAGGTGgCTCTGGCGGTGGCGGAAGTGCACTTTCTTCTGAGCTGACTCAGGaCCCT | |
| GATGTGTCTATGGCCTTGGGTCAGACAGTCACCATTTCATGCCGAGGAGACAGCCTC | |
| AAAAGATTTTATGCAAGTTGGTATCACCAGAAGCCAGGACAGGCCCCTGTCCTTGTC | |
| TTCTATGGTAAAGAAAATCGGCCCTCAGGGATCCCAGACCGGTTCTCTGGCTCCGAC | |
| TCTGGAGACACAGCCTCCTTGACCATCACTGGGGCTCAGGCGGAAGATGAGGGTGAC | |
| TATTACTGTCACACTCAGGACACCAGTGCTCGCCAATATGTCTTCGGGAGTGGGACC | |
| AAGGTCACCGTCCTAGGT, | |
| PINT 11A3 | |
| GAGGTGCAGCTGGTGCAGTCGGGCGCTGAGGTGAAGAAGCCTGGGGCCTCAGTGAAG | SEQ ID NO:29 |
| GTCTCCTGTAAGGCCTCTGGTTACTCTTTTACCAACTATGGTCTCAACTGGGTGCGA | |
| CAGGCCCCTGGACAGGGACTTGAGTGGATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTCACGACACCGCCGTTTATTAC | |
| TGTGCGAGACAGATTTTTTCTCATTGTACTGGTGGCAGTTGCTACCCTTTTGACTCC | |
| TGGGGCCGAGGCACCCTGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAAC | |
| TACTATGCAAGTTGGTACCAGCAGAAGCCAGGGCAGGCCCCTCTCCTTGTCATGTTT | |
| GGTAAGAACAACCGGCCCTCAGAGATCCCAGGCCGATTCTCTGGCTCCAGTTCGGGA | |
| AACACAGCTTCCTTGACCATCACTGGGGCTCAGGCGGAAGATGAGGCTGACTATTAC | |
| TGTAATTCTCGAGACAGAAACAGTCATCAATGGGTGTTCGGCGGAGGGACCAAGCTG | |
| ACCGTCCTAGGT, | |
| PINT 11A4 | |
| GAGCTGCAGCTGTTGGAGTCTGGGGGAGGCTTGGTACAGCCTGGGGGGTCCCTGAGA | SEQ ID NO:30 |
| CTCTCCTGTGCAGCCTCTCGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGTAGTCCCTATAGCAGCAGGTGGTACTCGTTCGACCCCTGGGGCCAAGGG | |
| ACAATGGTCACCGTCTCGAGTGGAGGCGGCGGTTCAGGCGCAGGTGGCTCTGGCGGT | |
| GGCGGAAGTGCACTTTCCTATGAGCTGACTCAGCCACCCTCAGTGTCCGTGTCCCCA | |
| GGACAGACAGCCACCATCACCTGCTCTGGAGATGACTTGGGGAATAAATATGTTTCG | |
| TGGTATCAACAGAAGCCAGGCCAGTCCCCTGTGCTGGTCATCTATCAAGATACCAAG | |
| CGGCCCTCAGGGATCCCTGAGCGATTCTCTGGCTCCAACTCTGGGAACATAGCCACT | |
| CTGACCATCAGCGCGACCCAGGCTGTGGATGAGGCTCACTATTATTGTCAGGTGTGG | |
| GACACCGGCACTGTGGTTTTCGGCGGCGGGACCAAGCTGACCGTCCTAGGT, | |
| PINT 11A5 | |
| CAGGTCCAGCTGGTGCAGTCTGGGGCTGAGGTGAAGAAGCCTGGGGCCTCAGTGAAG | SEQ ID NO:31 |
| GTCTCCTGTAAGGCCTCTGGTTACTCTTTTACCAACTATGGTCTCAACTGGGTGCGA | |
| CAGGCCCCTCGACAGGGACTTGAGTGGATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTGACGACACCGCCGTTTATTAC | |
| TGTGCGAGAGAGATTTTTTCTCATTGTACTGGTGGCAGTTGCTACCCTTTTGACTCC | |
| TGGGGCAAAGGAACCCTGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATCATTGGGTGTTCGGCGGACGGACCAAG | |
| GTCACCGTCCTAGGT, | |
| PINT 11A7 | |
| GAGGTCCAGCTGGTGCAGTCTGGGGCTGAGGTGAAGAAGCCTGGGGCCTCAGTGAAG | SEQ ID NO:32 |
| GTCTCCTGTAAGGCCTCTGGTTACTCTTTTACCAACTATGGTCTCGACTGGGTGCGA | |
| CAGGCCCCTGGACAGGGACTTGAGTGGATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTGACGACACCGCCGTTTATTAC | |
| TGTGCGAGAGAGATTTTTTCTCATTGTACTGGTGGCAGTTGCTACCCTTTTGACTCC | |
| TGGGGCAGAGGGACAATGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCATCGGAATTGGGTGTTCGGCGGAGGGACC | |
| AAGGTCACCGTCCTAGGT, | |
| PINT 11A11 | |
| CAGGTGCAGCTGGTGGAGTCTGGGGGAGGCCTGGTCAAGCCTGGGGGGTCCCTGAGA | SEQ ID NO:33 |
| CTCTCCTGTGCAGCCTCTGGATTCACCTTCAGCAGCCACACCATGAACTGGGTCCGC | |
| CAGGCTCAAGGGAAGGGGCTGGAGTGGGTCTCATCCATTAGTGGTAGTGGTCGTTAC | |
| ATTTACTATTCAGACTCAGTCAAGGGCCGGTTCACCATCTCCACAGACGCCGCCAAG | |
| AACTCTCTGTATCTGCAAATGAACAACCTGAGAGCCGAGGACACGGCTGTCTATTAC | |
| TGTACCAGAGCGAAATTCGGTGACTACCTCTTTGACTCCTGGGGCCAGGGCACCCTG | |
| GTCACCGTCTCGAGTGCAGGCGGCGGTTCAGGCGGAGGTGGCTCTGGCGGTGGCGGA | |
| AGTGCACTTAATTTTATGCTGACTCAGCCCCACTCTGTGTCGCAGTCTCCGGGGAAG | |
| ACGGTAACCATCTCCTGCACCCGCAGTAGTGGCAGAATTGCCAGCAACTTTGTGCAG | |
| TGGTACCAGCAGCGCCCGGGCAGTGCCCCCACCACTGTGATCTATGAGGATAACCGA | |
| CGACCCTCTGGGGTCCCTGATCGGTTCTCTGGCTCCATCGACAGCTCCTCCAACTCT | |
| GCCTCCCTCACCATCTCTGGACTAAAGACTGAGGACGACGCTGACTACTATTGTCAG | |
| TCTTATGATGCCAGATATCAAGTCTTCGGAACTGGGACCAAGGTCACCGTCCTAGGG, | |
| PINT 11A12 | |
| CAGGTGCAGCTGTTGGAGTCTGGGGGAGGCTTGGTACAGCCTGGGGGGTCCCTGAGA | SEQ ID NO:34 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGCGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCCAGGTCGCCTGTCCCGCCGTGGGCGGACTGGTACTACTTTGATTATTGGGGC | |
| CGGGGGACAATGGTCACCGTCTCGAGTGGAGGCGGCGGTTCAGGCGGAGGTGGCTCT | |
| GGCGGTGGCGGAAGTGCACAGGCTGTGCTGACTCAGCCGTCCTCAGTGTCTGGGGCC | |
| CCAGGGCAGAGGGTCACCATCTCCTGCACTGGGAGCAGGTCCAACTTCGGGGCAGGT | |
| TATGATGTACACTGGtACCAGCAGTTTCCAGGAACAGCCCCCAAACTCCTCATCTAT | |
| GGTAACACCAATCGGCCCTCAGGGGTCCCTGACCGATTCTCTGGCTCCAGGTCTGGC | |
| ACCTCAGCCTCCCTGGCCATCACTGGGCTCCAGGCTGAGGATGAGGCTGATTATTAC | |
| TGCCAGTCATATGACAGCAACCTGAGTGGTTCGGTGTTCGGCGGCGGGACCAAGGTC | |
| ACCGTCCTAGGT, | |
| PINT 12A1 | |
| GAGGTCCAGCTGGTACAGTCTGGAGCTGAGGTGAAGAAGCCTGGGGCCTCAGTGAAG | SEQ ID NO:35 |
| GTCTCCTGTAAGGCCTCTGGTTACTCTTTTACCAACTATGGTCTCAACTGGGTGCGA | |
| CAGGCCCCTGGACAGGGACTTGAGTGGATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTGACGACACCGCCGTTTATTAC | |
| TGTGCGAGACAGATTTTTTCTCATTGTACTGGTGGCAGTTGCTACCCTTTTGACTCC | |
| TGGGGCAAAGGAACCCTGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAAC | |
| TATTATGCAAGCTGGTACCAGCAGAAGCCAGGGCAGGCCCCTGTCCTTGTCCTCTAC | |
| AGTAAAAACAGCCGGCCCTCTGGGGTCCCAGACCGATTCTCTGGCTCCAGCTCAgGA | |
| ACCACAGCTTCCTTGACAATCAGTGGGGCTCAGGCGGAAGATGAgGCTGACTATTAC | |
| TGTAATTCTCGGGACACCAGTGGTGACCTTCGCTGCGTGTTCGCCGGAGGCACCAAG | |
| CTGACCGTCCTAGGT, | |
| PINT 12A2 | |
| GAGGTCCAGCTGGTGCAGTCTGGGGCTGAGGTGAAGAAGCCTGGGGCCTCAGTGAAG | SEQ ID NO:36 |
| GTCTCCTGTAAGGCCTCTGGTTACTCTTTTACCAACTATGGTCTCAACTGGGTGCGA | |
| CAGGCCCCTGCACAGGGACTTGAGTGCATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTGACGACACCGCCGTTTATTAC | |
| TGTGCGAGAGAGATTTTTTCTCATTGTACTGGTGGCAGTTGCTACCCTTTTGACTCC | |
| TGGGGCCAGGGCACCCTGGTCACCGTCTCGAGTGGAGGCGGCGGTTCAGGCGGAGGT | |
| GGCTCTGGCGGTGGCGCAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAAC | |
| TACTATGCAAGTTGGTACCAGCAGAAGCCAGGGCAGGCCCCTCTCCTTGTCATGTTT | |
| GGTAAGAACAACCGGCCCTCAGACATCCCAGGCCGATTCTCTGGCTCCAGTTCGGGA | |
| AACACAGCTTCCTTGACCATCACTGGGGCTCAGGCGGAAGATGAGGCTGACTATTAC | |
| TGTAATTCTCCAGACAGTAACAGTCATCAATGGGTGTTCGGCGGAGGGACCAAGCTG | |
| ACCGTCCTAGGT, | |
| PINT 12A3 | |
| CAGGTGCAGCTGGTGCAGTCTGGGGCTGAGGTGAAGAAGCCTGGGGCCTCAGTGAAG | SEQ ID NO:37 |
| GTCTCCTGTAAGGCCTCTGGTTACTCTTTTACCAACTATGGTCTCAACTGGGTGCGA | |
| CAGGCCCCTGGACAGGGACTTGAGTGGATGGGATGGATCAGCCCTTACACTGGTTAC | |
| ACAAATTATGCACAGAAGTTCCAGGGCAGAGTCACCATGACTTCAGATAAATCCACG | |
| AGCACAGCCTACATGGACCTGAGGAGTCTGAGATCTGACGACACGGCCATTTATTAT | |
| TGTGCGAGAGAGATTTTCTCCCATTGTAGTGGTGGTAGTTGCTACCCTTTTGACTAC | |
| TGGGGCCAGGGAACCCTGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTCTACTTGTCATCTAT | |
| GGTAGAAACAACCGGcCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTACTAACCATGGGAATTGGGTGTTCGGCGGAGGGACC | |
| CAGCTCACCGTTTTAAGT, | |
| and | |
| PINT 12A4 | |
| CAGGTCCAGCTGGTGCAGTCTGGGGCTGAGGTGAAGAAGCCTGGGgCcTCAGTGAAG | SEQ ID NO:38 |
| GTCTCCTGTAAGGCCTCTGGTTACTCTTTTACCAACTATGGTCTCAACTGGGTGCGA | |
| CAGGCCCCTGGACAGGGACTTGAGTGGATGGGATGGATCAGCCCTTACACTGGTTAC | |
| ACAAATTATGCACAGAAGTTCCAGGGCAGAGTCACCATGACCACAGATAAATCCACG | |
| AGCACAGCCTACATGGACCTGAGGAGTCTGAGATCTGACGACACCGCCGTTTATTAC | |
| TGTGCGAGAGAGATTTTTTCTCATTGTACTGGTGGCAGTTGCTACCCTTTTGACTCC | |
| TGGGGCAGGGGGACAATGGTCACCGTCTCGAGTGGAGGCGGCGGTTCAGGCGGAGGT | |
| GGCTCTGGCGGTGGCGGAAGTGCACTTTCTTCTGAGCTGACTCAGGACCCTGCTGTG | |
| TCTGTGGCCTTGGGACAGACAGTCAGGATCACATGCCAAGGAGACAGCCTCAGAAGC | |
| TATTATGCAAGCTGGTACCAGCAGAAGCCAGGACAGGCCCCTGTACTTGTCATCTAT | |
| GGTAAAAACAACCGGCCCTCAGGGATCCCAGACCGATTCTCTGGCTCCAGCTCAGGA | |
| AACACAGCTTCCTTGACCATCACTGGGGCTCAGGCGGAAGATGAGGCTGACTATTAC | |
| TGTAACTCCCGGGACAGCAGTGGTAACCTCAATTGGGTGTTCGGCGGAGGGACCCAG | |
| CTCACCGTTTTAAGT. | |

Inhibition of IGF-I and IGF-II Binding to IGF-IR

In another embodiment, the invention provides an IGF-IR antibody that inhibits the binding of IGF-I to IGF-IR and/or the binding of IGF-II to IGF-IR. In a preferred embodiment, the IGF-IR is human. In another preferred embodiment, the anti-IGF-IR antibody is a human antibody. In another embodiment, the antibody or portion thereof inhibits binding between IGF-IR and IGF-I and/or IGF-II with an IC₅₀ of no more than 100 nM. In a preferred embodiment, the IC₅₀ is no more than 10 nM. In a more preferred embodiment, the IC₅₀ is no more than 1 nM. The IC₅₀ can be measured by any method known in the art. Typically, an IC₅₀ can be measured by ELISA, RIA, or a cell-based assay where the antibody is assessed for its ability to inhibit binding of radiolabeled IGFs. In a preferred embodiment, the IC₅₀ is measured by a cell-based ligand competition binding assay.

In another embodiment, the invention provides an anti-IGF-IR antibody that prevents activation of the IGF-IR in the presence of IGF-I and/or IGF-II. In a preferred embodiment, the anti-IGF-IR antibody inhibits IGF-1R-induced tyrosine phosphorylation within the cytoplasmic domain of the beta IGF-1R subunit upon occupancy of the receptor. In a more preferred embodiment, the IGF-1R antibody inhibits IGF-1R-induced tyrosine phosphorylation that occurs at tyrosines 1131, 1135, and 1136 within the kinase domain of the IGF-1R beta subunit in response to extracellular binding of IGF-I and/or IGF-II. In another preferred embodiment, the IGF-IR antibody inhibits downstream cellular events from occurring. For instance, the anti-IGF-IR can inhibit tyrosine phosphorylation of Shc and insulin receptor substrate (IRS) 1 and 2, Akt 1 or Akt 2, Erk1/2, all of which are normally phosphorylated when cells are treated with IGF-I (Kim et al., J. Biol. Chem. 273: 4543-4550, 1998). One can determine whether an IGF-IR antibody can prevent activation of IGF-IR in the presence of IGF-I and/or IGF-II by determining the levels of tyrosine phosphorylation on the IGF-IR beta subunit by Western blot, immunoprecipitation, ELISA, or FACS.

In another aspect of the invention, the antibody causes the downregulation of IGF-IR from a cell treated with the antibody. In one embodiment, the IGF-IR is internalized into the endosomal pathway of the cell and catabolized. After the IGF-IR antibody binds to IGF-IR, the antibody bound to IGF-IR is internalized. One may measure the downregulation of IGF-IR by any method known in the art including immunoprecipitation, confocal microscopy, or Western blot. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, or comprises a heavy chain, light chain or antigen-binding region thereof.

Activation of IGF-IR by IGF-IR Antibody Binding

Another aspect of the present invention involves activating IGF-IR antibodies. An activating antibody differs from an inhibiting antibody because it amplifies or substitutes for the effects of IGF-I and IGF-II on IGF-IR. In one embodiment, the activating antibody is able to bind to IGF-IR and cause it to be activated in the absence of IGF-I and IGF-II. This type of activating antibody is essentially a partial or complete mimetic of IGF-I and IGF-II. In another embodiment, the activating antibody amplifies the effect of IGF-I and IGF-II on IGF-IR.

This type of antibody does not activate IGF-IR by itself, but rather increases the activation of IGF-IR in the presence of IGF-I and IGF-II. A mimic anti IGF-IR antibody may be easily distinguished from an amplifying IGF-IR antibody by treating cells in vitro with an antibody in the presence or absence of low levels of IGF-I and IGF-II. If the antibody is able to cause IGF-IR activation in the absence of IGF-I and IGF-II, e.g., it increases IGF-IR tyrosine phosphorylation, and then the antibody is a mimic antibody. If the antibody cannot cause IGF-IR activation in the absence of IGF-I and IGF-II but is able to amplify the amount of IGF-IR activation, then the antibody is an amplifying antibody.

Inhibition of IGF-IR Tyrosine Phosphorylation IGF-IR Levels and Tumor Cell Growth In Vivo by IGF-IR Antibodies

Another embodiment of the invention provides an IGF-IR antibody that inhibits IGF-IR tyrosine phosphorylation and receptor levels in vivo. In one embodiment, administration of IGF-IR antibody to an animal causes a reduction in IGF-IR phosphotyrosine signal in IGF-IR-expressing tumors. In a preferred embodiment, the IGF-IR antibody causes a reduction in phosphotyrosine signal by at least 20%. In a more preferred embodiment, the IGF-IR antibody causes a decrease in phosphotyrosine signal by at least 50%, more preferably 60%. In an even more preferred embodiment, the antibody causes a decrease in phosphotyrosine signal of at least 70%, more preferably 80%, even more preferably 90%. In a preferred embodiment, the antibody is administered approximately 24 hours before the levels of tyrosine phosphorylation are measured.

The levels of tyrosine phosphorylation may be measured by any method known in the art, such as those described infra. See, e.g., Example 5 and FIGS. 4 & 6. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, or comprises a heavy chain, light chain or antigen-binding portion thereof.

In another embodiment, administration of IGF-IR antibody to an animal causes a reduction in IGF-IR levels in IGF-IR-expressing tumors. In a preferred embodiment, the IGF-IR antibody causes a reduction in receptor levels by at least 20% compared to an untreated animal. In a more preferred embodiment, the IGF-IR antibody causes a decrease in receptor levels to at least 50%, more preferably 60% of the receptor levels in an untreated animal. In an even more preferred embodiment, the antibody causes a decrease in receptor levels by at least 70%, more preferably 80%. In a preferred embodiment, the antibody is administered approximately 24 hours before the IGF-IR levels are measured. The IGF-IR levels may be measured by any method known in the art, such as those described infra. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 or comprises a heavy chain, light chain or antigen-binding portion thereof.

In another embodiment, an IGF-IR antibody inhibits tumor cell growth in vivo. The tumor cell may be derived from any cell type including, without limitation, epidermal, epithelial, endothelial, leukemia, sarcoma, multiple myeloma, or mesodermal cells. Examples of common tumor cell lines for use in xenograft tumor studies include A549 (non-small cell lung carcinoma) cells, DU-145 (prostate) cells, MCF-7 (breast) cells, Colo 205 (colon) cells, 3T3/]GF-IR (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SY5Y cells, Sk-Mel-2 cells, NCI-H929, RPM18226, and A431 cells. In a preferred embodiment, the antibody inhibits tumor cell growth as compared to the growth of the tumor in an untreated animal. In a more preferred embodiment, the antibody inhibits tumor cell growth by 50%. In an even more preferred embodiment, the antibody inhibits tumor cell growth by 60%, 65%, 70%, or 75%. In one embodiment, the inhibition of tumor cell growth is measured at least 7 days after the animals have started treatment with the antibody. In a more preferred embodiment, the inhibition of tumor cell growth is measured at least 14 days after the animals have started treatment with the antibody. In another preferred embodiment, another antineoplastic agent is administered to the animal with the IGF-IR antibody. In a preferred embodiment, the antineoplastic agent is able to further inhibit tumor cell growth. In an even more preferred embodiment, the antineoplastic agent is adriamycin, taxol, tamoxifen, 5-fluorodeoxyuridine (5-FU) or CP-358,774. In a preferred embodiment, the co-administration of an antineoplastic agent and the IGF-IR antibody inhibits tumor cell growth by at least 50%, more preferably 60%, 65%, 70% or 75%, more preferably 80%, 85% or 90% after a period of 22-24 days.

Induction of Apoptosis by IGF-IR Antibodies

Another aspect of the invention provides an IGF-IR antibody that induces cell death. In one embodiment, the antibody causes apoptosis. The antibody may induce apoplosis either in vivo or in vitro. In general, tumor cells are more sensitive to apoptosis than normal cells, such that administration of an IGF-IR antibody causes apoptosis of a tumor cell preferentially to that of a normal cell. In another embodiment, the administration of an IGF-IR antibody effects the activation of a serine-threonine kinase Akt, which is involved in the phosphatidyl inositol (PI) kinase pathway.

The PI kinase pathway, in turn, is involved in the cell proliferation and prevention of apoptosis. Thus, inhibition of Akt can cause apoptosis. In a more preferred embodiment, the antibody is administered in vivo to cause apoptosis of an IGF-I and IGF-II expressing cell. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, or comprises a heavy chain, light chain, or antigen-binding portion thereof.

Methods of Producing Antibodies and Antibody-Producing Cell Lines

Immunization

In one embodiment of the instant invention, human antibodies are produced by immunizing a non-human animal comprising some or the entire human immunoglobulin locus with an IGF-IR antigen. In a preferred embodiment, the non-human animal is a XENOMOUSE™, which is an engineered mouse strain that comprises large fragments of the human immunoglobulin loci and is deficient in mouse antibody production. See, e.g. Green et al. Nature Genetics 7: 13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00/09560, published Feb. 24, 2000 and WO 00/037504, published Jun. 29, 2000. The XENOMOUSE™ produces an adult-like human repertoire of fully human antibodies, and generates antigen specific human Mabs. A second generation XENOMOUSE™ contains approximately 80% of the human antibody repertoire through introduction of megabase sized, germline configuration YAC fragments of the human heavy chain loci and κ light chain loci. See Mendez et al. Nature Genetics 15:146-156 (1997), Green and Jakobovits J. Exp. Med. 188:483-495 (1998), the disclosures of which are hereby incorporated by reference.

The invention also provides a method for making IGF-IR antibodies from non-human, non-mouse animals by immunizing non-human transgenic animals that comprise human immunoglobulin loci. One may produce such animals using the methods described immediately above. The methods disclosed in these patents may be modified as described in U.S. Pat. No. 5,994,619. In a preferred embodiment, the non-human animals may be rats, sheep, pigs, goats, cattle, or horses. In another embodiment, the non-human animal comprising human immunoglobulin gene loci are animals that have a “minilocus” of human immunoglobulins. In the minilocus approach, an exogenous Ig locus is mimicked through the inclusion of individual genes from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs including portions of the Ig locus can be generated and introduced into animals. However, a potential disadvantage of the minilocus approach is that there may not be sufficient immunoglobulin diversity to support full B-cell development, such that there may be lower antibody production.

In order to produce a human IGF-IR antibody, a non-human animal comprising some or all of the human immunoglobulin loci is immunized with an IGF-IR antigen and the antibody or the antibody-producing cell is isolated from the animal. The IGF-IR antigen may be isolated and/or purified IGF-IR and is preferably a human IGF-IR. In another embodiment, the IGF-IR antigen is a fragment of IGF-IR, preferably the extracellular domain of IGF-IR. In another embodiment, the IGF-IR antigen is a fragment that comprises at least one epitope of IGF-IR. In another embodiment, the IGF-IR antigen is a cell that expresses IGF-IR on its cell surface, preferably a cell that overexpresses IGF-IR on its cell surface.

Immunization of animals may be done by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow, Lane supra, and U.S. Pat. No. 5,994,619. In a preferred embodiment, the IGF-IR antigen is administered with an adjuvant to stimulate the immune response.

Such adjuvants include complete or incomplete Freund's adjuvant, RIBI (muramyl dipeptides), or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

Production of Antibodies and Antibody-Producing Cell Lines

After immunization of an animal with an IGF-IR antigen, antibodies and/or antibody-producing cells may be obtained from the animal. An IGF-IR antibody-containing serum is obtained from the animal by bleeding or sacrificing the animal. The serum may be used as it is obtained from the animal, an immunoglobulin fraction may be obtained from the serum, or the IGF-IR antibodies may be purified from the serum. Serum or immunoglobulins obtained in this manner are polyclonal, which are disadvantageous because the amount of antibodies that can be obtained is limited and the polyclonal antibody has a heterogeneous array of properties. In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using IGF-IR, a portion thereof, or a cell expressing IGF-IR. In a preferred embodiment, the initial screening is performed using an enzyme-linked immunoassay (ELISA) or a radioimmunoassay (RIA), preferably an ELISA. An example of ELISA screening is provided in WO 00/37504, herein incorporated by reference.

In another embodiment, antibody-producing cells may be prepared from a human who has an autoimmune disorder and who expresses IGF-IR antibodies. Cells expressing the IGF-IR antibodies may be isolated by isolating white blood cells and subjecting them to fluorescence activated cell sorting (FACS) or by panning on plates coated with IGF-IR or a portion thereof. These cells may be fused with a human non-secretory myeloma to produce human hybridomas expressing human IGF-IR antibodies. In general, this is a less preferred embodiment because it is likely that the IGF-IR antibodies will have a low affinity for IGF-IR.

IGF-IR antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro.

Methods of selecting, cloning and expanding hybridomas are well known to those of ordinary skill in the art.

Preferably, the immunized animal is a non-human animal that expresses human immunoglobulin genes and the splenic B cells are fused to a myeloma derived from the same species as the non-human animal. More preferably, the immunized animal is a XENOMOUSE™ and the myeloma cell line is a non-secretory mouse myeloma, such as the myeloma cell line is NSO-bcl-2.

In one aspect, the invention provides hybridomas are produced that produce human IGF-IR antibodies. In a preferred embodiment, the hybridomas are mouse hybridomas, as described above. In another preferred embodiment, the hybridomas are produced in a non-human, non-mouse species such as rats, sheep, pigs, goats, cattle, or horses. In another embodiment, the hybridomas are human hybridomas, in which a human non-secretory myeloma is fused with a human cell expressing a IGF-IR antibody.

Nucleic Acids, Vectors, Host Cells, and Recombinant Methods of Making Antibodies

Nucleic Acids

Nucleic acid molecules encoding IGF-IR antibodies of the invention are provided. In one embodiment, the nucleic acid molecule encodes a heavy and/or light chain of an IGF-IR immunoglobulin. In a preferred embodiment, a single nucleic acid molecule encodes a heavy chain of an IGF-IR immunoglobulin and another nucleic acid molecule encodes the light chain of an IGF-IR immunoglobulin. In a more preferred embodiment, the encoded immunoglobulin is a human immunoglobulin, preferably a human IgG. The encoded light chain may be a λ chain or a κ chain, preferably a λ chain.

The nucleic acid molecule encoding the variable region of the light chain may be derived from the A30, A27, or O12 Vκ gene. In another preferred embodiment, the nucleic acid molecule encoding the light chain comprises the joining region derived from Jκ1, Jκ2, or Jκ4. In an even more preferred embodiment, the nucleic acid molecule encoding the light chain contains no more than ten amino acid changes from the germline, preferably no more than six amino acid changes, and even more preferably no more than three amino acid changes.

The invention provides a nucleic acid molecule that encodes a variable region of the light chain (VL) containing at least three amino acid changes compared to the germline sequence, wherein the amino acid changes are identical to the amino acid changes from the germline sequence from the VL of one of the antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. The invention also provides a nucleic acid molecule comprising a nucleic acid sequence that encodes the amino acid sequence of the variable region of the light chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. The invention also provides a nucleic acid molecule comprising a nucleic acid sequence that encodes the amino acid sequence of one or more of the CDRs of any one of the light chains of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In a preferred embodiment, the nucleic acid molecule comprises a nucleic acid sequence that encodes the amino acid sequence of all of the CDRs of any one of the light chains of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In another embodiment, the nucleic acid molecule comprises a nucleic acid sequence that encodes the VL amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19, or comprises a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, or SEQ ID NO:38 or a fragment thereof.

In another preferred embodiment, the nucleic acid molecule comprises a nucleic acid sequence that encodes the amino acid sequence of one or more of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or comprises a nucleic acid sequence of one or more of the CDRs of any one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38. In a more preferred embodiment, the nucleic acid molecule comprises a nucleic acid sequence that encodes the amino acid sequence of all of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or comprises a nucleic acid sequence of all the CDRs of any one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, or SEQ ID NO:38. The invention also provides a nucleic acid molecules that encodes an amino acid sequence of a VL that has an amino acid sequence that is at least 70%, 75%, 80%, 85%, 90%, 95%, 96%, 97%, 98% or 99% identical to a VL described above, particularly to a VL that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. The invention also provides a nucleic acid sequence that is at least 70%, 75%, 80%, 85%, 90%, 95%, 96%, 97%, 98% or 99% identical to a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38 or a fragment thereof. In another embodiment, the invention provides a nucleic acid molecule encoding a VL that hybridizes under highly stringent conditions to a nucleic acid molecule encoding a VL as described above, particularly a nucleic acid molecule that comprises a nucleic acid sequence encoding a VL amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19. The invention also provides a nucleic acid sequence encoding an VL that hybridizes under highly stringent conditions to a nucleic acid molecule comprising a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38 or a nucleic acid sequence that would hybridize except for the degeneracy of the genetic code.

The invention also provides a nucleic acid molecule encoding the variable region of the heavy chain (VH) is derived from the DP-14, DP-47, DP-50, DP-73, or DP-77 VH gene. In another embodiment, the nucleic acid molecule encoding the VH comprises the joining region derived from JH6 or JH5. In another preferred embodiment, the D segment is derived from 3-3, 6-19 or 4-17. In an even more preferred embodiment, the nucleic acid molecule encoding the VH contains no more than ten amino acid changes from the germline gene, preferably no more than six amino acid changes, and even more preferably no more than three amino acid changes. In a highly preferred embodiment, the nucleic acid molecule encoding the VH contains at least one amino acid change compared to the germline sequence, wherein the amino acid change is identical to the amino acid change from the germline sequence from the heavy chain of one of the antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In an even more preferred embodiment, the VH contains at least three amino acid changes compared to the germline sequences, wherein the changes are identical to those changes from the germline sequence from the VH of one of the antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5.

In one embodiment, the nucleic acid molecule comprises a nucleic acid sequence that encodes the amino acid sequence of the VH of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 or a fragment of any one thereof. In a preferred embodiment, the nucleic acid molecule comprises a nucleic acid sequence that encodes the amino acid sequence of PINT-7A4, PINT-8A1, PINT-9A2, PINT-11A1, and PINT-11A4 or a fragment of any one thereof. In a preferred embodiment, the nucleic acid molecule comprises a nucleic acid sequence that encodes the amino acid sequence of PINT-8A 1, PINT-9A2, and PINT-11A4 or a fragment of any one thereof. Table 2 shows the nucleic acid sequences of the scFvs PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5.

In another embodiment, the nucleic acid molecule comprises a nucleic acid sequence that encodes the amino acid sequence of one or more of the CDRs of the heavy chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, or PINT-12A5. In a preferred embodiment, the nucleic acid molecule comprises a nucleic acid sequence that encodes the amino acid sequences of all of the CDRs of the heavy chain of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-1A5, PINT-11A7, PINT-11A12, PINT-12A1, PINT-12A2, PINT-12A3, PINT-12A4, or PINT-12A5. In another preferred embodiment, the nucleic acid molecule comprises a nucleic acid sequence that encodes the VH amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or that comprises a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38. In another preferred embodiment, the nucleic acid molecule comprises a nucleic acid sequence that encodes the amino acid sequence of one or more of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or comprises a nucleic acid sequence of one or more of the CDRs of any one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38. In a preferred embodiment, the nucleic acid molecule comprises a nucleic acid sequence that encodes the amino acid sequences of all of the CDRs of any one SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19, or comprises a nucleic acid sequence of all of the CDRs of any one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38.

In another embodiment, the nucleic acid molecule encodes an amino acid sequence of a VH that is at least 70%, 75%, 80%, 85%, 90%, 95%, 96%, 97%, 98% or 99% identical to one of the amino acid sequences encoding a VH as described immediately above, particularly to a VH that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19. The invention also provides a nucleic acid sequence that is at least 70%, 75%, 80%, 85%, 90%, 95%, 96%, 97%, 98% or 99% identical to a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, or SEQ ID NO:38. In another embodiment, the nucleic acid molecule encoding a VH is one that hybridizes under highly stringent conditions to a nucleic acid sequence encoding a VH as described above, particularly to a VH that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19. The invention also provides a nucleic acid sequence encoding a VH that hybridizes under highly stringent conditions to a nucleic acid molecule comprising a nucleic acid sequence of one of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38 or a nucleic acid sequence that would hybridize except for the degeneracy of the genetic code.

The nucleic acid molecule encoding either or both of the entire heavy and light chains of an IGF-IR antibody or the variable regions thereof may be obtained from any source that produces an IGF-IR antibody. Methods of isolating mRNA encoding an antibody are well known in the art. See, e.g., Sambrook et al. The mRNA may be used to produce cDNA for use in the polymerase chain reaction (PCR) or cDNA cloning of antibody genes. In one embodiment of the invention, the nucleic acid molecules may be obtained from a hybridoma that expresses an IGF-IR antibody, as described above, preferably a hybridoma that has as one of its fusion partners a transgenic animal cell that expresses human immunoglobulin genes, such as a XENOMOUSE™, non-human mouse transgenic animal or a nonhuman, non-mouse transgenic animal. In another embodiment, the hybridoma is derived from a non-human, non-transgenic animal, which may be used, e.g., for humanized antibodies.

A nucleic acid molecule encoding the entire heavy chain of a IGF-IR antibody may be constructed by fusing a nucleic acid molecule encoding the variable domain of a heavy chain or an antigen-binding domain thereof with a constant domain of a heavy chain. Similarly, a nucleic acid molecule encoding the light chain of a IGF-IR antibody may be constructed by fusing a nucleic acid molecule encoding the variable domain of a light chain or an antigen-binding domain thereof with a constant domain of a light chain. The nucleic acid molecules encoding the VH and VL chain may be converted to full-length antibody genes by inserting them into expression vectors already encoding heavy chain constant and light chain constant regions, respectively, such that the VH segment is operatively linked to the heavy chain constant region (CH) segment(s) within the vector and the VL segment is operatively linked to the light chain constant region (CL) segment within the vector.

Alternatively, the nucleic acid molecules encoding the VH or VL chains are converted into full-length antibody genes by linking, e.g., ligating the nucleic acid molecule encoding a VH chain to a nucleic acid molecule encoding a CH chain using standard molecular biological techniques. The same may be achieved using nucleic acid molecules encoding VL and CL chains. The sequences of human heavy and light chain constant region genes are known in the art. See, e.g., Kabat et al., Sequences of Proteins of Immunological Interest, 5th Ed., NIH Publ. No.91-3242, 1991. Nucleic acid molecules encoding the full-length heavy and/or light chains may then be expressed from a cell into which they have been introduced and the IGF-IR antibody isolated.

In a preferred embodiment, the nucleic acid encoding the variable region of the heavy chain encodes the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19, and the nucleic acid molecule encoding the variable region of the light chains encodes the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

In another embodiment, a nucleic acid molecule encoding either the heavy chain of an IGF-IR antibody or an antigen-binding domain thereof, or the light chain of an IGF-IR antibody or an antigen-binding domain thereof may be isolated from a non-human, non-mouse animal that expresses human immunoglobulin genes and has been immunized with an IGF-IR antigen. In other embodiment, the nucleic acid molecule may be isolated from an IGF-IR antibody-producing cell derived from a non-transgenic animal or from a human patient who produces IGF-IR antibodies. Methods of isolating mRNA from the IGF-IR antibody producing cells may be isolated by standard techniques, cloned and/or amplified using PCR and library construction techniques, and screened using standard protocols to obtain nucleic acid molecules encoding IGF-IR heavy and light chains.

The nucleic acid molecules may be used to recombinantly express large quantities of IGF-IR antibodies, as described below. The nucleic acid molecules may also be used to produce chimeric antibodies, single chain antibodies, immunoadhesins, diabodies, mutated antibodies and antibody derivatives, as described further below. If the nucleic acid molecules are derived from a non-human, non-transgenic animal, the nucleic acid molecules may be used for antibody humanization, also as described below.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, initer alia, to isolate nucleic acid sequences for use in producing variable domains of IGF-IR antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

Vectors

The invention provides vectors comprising the nucleic acid molecules of the invention that encode the heavy chain or the antigen-binding portion thereof. The invention also provides vectors comprising the nucleic acid molecules of the invention that encode the light chain or antigen-binding portion thereof. The invention also provides vectors comprising nucleic acid molecules encoding fusion proteins, modified antibodies, antibody fragments, and probes thereof.

To express the antibodies, or antibody portions of the invention, DNAs encoding partial or full-length light and heavy chains, obtained as described above, are inserted into expression vectors such that the genes are operatively linked to transcriptional and translational control sequences. Expression vectors include plasmids, retroviruses, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present). A convenient vector is one that encodes a functionally complete human CH or CL immunoglobulin sequence, with appropriate restriction sites engineered so that any VH or VL sequence can be easily inserted and expressed, as described above.

In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding 10 regions. The recombinant expression vector can also encode a signal peptide that facilitates secretion of the antibody chain from a host cell. The antibody chain gene may be cloned into the vector such that the signal peptide is linked inframe to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399 216, 4,634,665, and 5,179,017, all by Axel et al.). For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr- host cells with methotrexate selection/amplification) and the neo gene (for G418 selection).

Non-Hybridoma Host Cells and Methods of Recombinantly Producing Protein

Nucleic acid molecules encoding the heavy chain or an antigen binding portion thereof and/or the light chain or an antigen-binding portion thereof of an IGF-IR antibody, and vectors comprising these nucleic acid molecules, can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, and encapsulation of the polynucleotide(s) in liposomes, biolistic injection, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (which patents are hereby incorporated herein by reference).

Mammalian cell lines available as hosts for expression are well known in the art and include many immortalized cell lines available from the American Type Culture Collection (ATCC). These include, inter aria, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and a number of other cell lines. Mammalian host cells include human, mouse, rat, dog, monkey, pig, goat, bovine, horse, and hamster cells. Cell lines of particular preference are selected through determining which cell lines have high expression levels. Other cell lines that may be used are insect cell lines, such as Sf9 cells, amphibian cells, bacterial cells, plant cells, and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

Transgenic Animals

The invention also provides transgenic non-human animals comprising one or more nucleic acid molecules of the invention that may be used to produce antibodies of the invention. Antibodies can be produced in and recovered from tissue or bodily fluids, such as milk, blood or urine, of goats, cows, horses, pigs, rats, mice, rabbits, hamsters or other mammals. See, e.g., U.S. Pat. Nos. 5,827,690, 5,756,687, 5,750,172, and 5,741,957. As described above, non-human transgenic animals that comprise human immunoglobulin loci can be produced by immunizing with IGF-IR or a portion thereof.

In another embodiment, non-human transgenic animals are produced by introducing one or more nucleic acid molecules of the invention into the animal by standard transgenic techniques. See Hogan, sierra. The transgenic cells used for making the transgenic animal can be embryonic stem cells or somatic cells. The transgenic non-human organisms can be chimeric, non-chimeric heterozygotes, and non-chimeric homozygotes. See, e.g., Hogan et al., Manipulating the Mouse Embryo: A Laboratory Manual 2 ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999). In another embodiment, the transgenic non-human organisms may have a targeted disruption and replacement that encodes a heavy chain and/or a light chain of interest. In a preferred embodiment, the transgenic animals comprise and express nucleic acid molecules encoding heavy and light chains that bind specifically to IGF-IR, preferably human IGF-IR. In another embodiment, the transgenic animals comprise nucleic acid molecules encoding a modified antibody such as a single-chain antibody, a chimeric antibody or a humanized antibody. The IGF-IR antibodies may be made in any transgenic animal. In a preferred embodiment, the nonhuman animals are mice, rats, sheep, pigs, goats, cattle, or horses. The non-human transgenic animal expresses said encoded polypeptides in blood, milk, urine, saliva, tears, mucus, and other bodily fluids.

Phage Display Libraries

The invention provides a method for producing an IGF-IR antibody or antigen-binding portion thereof comprising the steps of synthesizing a library of human antibodies on phage, screening the library with a IGF-IR or a portion thereof, isolating phage that bind IGF-IR, and obtaining the antibody from the phage. One method to prepare the library of antibodies comprises the steps of immunizing a non-human host animal comprising a human immunoglobulin locus with IGF-IR or an antigenic portion thereof to create an immune response, extracting cells from the host animal the cells that are responsible for production of antibodies; isolating RNA from the extracted cells, reverse transcribing the RNA to produce cDNA, amplifying the cDNA using a primer, and inserting the cDNA into phage display vector such that antibodies are expressed on the phage. Recombinant IGF-IR antibodies of the invention may be obtained in this way.

Recombinant IGF-IR human antibodies of the invention in addition to the IGF-IR antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibody. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

In a preferred embodiment, to isolate human IGF-IR antibodies with the desired characteristics, a human IGF-IR antibody as described herein is first used to select human heavy and light chain sequences having similar binding activity toward IGF-IR, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature 348:552-554 (1990); and Griffiths et al., EMBO J 12:725-734 (1993). The scFv antibody libraries preferably are screened using human IGF-IR as the antigen.

Once initial human VL and VH segments are selected, “mix and match” experiments, in which different pairs of the initially selected VL and VH segments are screened for IGF-IR binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to IGF-IR.

Following screening and isolation of an IGF-IR antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

Class Switching

Another aspect of the instant invention is to provide a mechanism by which the class of an IGF-IR antibody may be switched with another. In one aspect of the invention, a nucleic acid molecule encoding VL or VH is isolated using methods well known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, an IGF-IR antibody that was originally IgM may be class switched to an IgG. Further, the class switching may be used to convert one IgG subclass to another, e.g., from IgG1 to IgG2. A preferred method for producing an antibody of the invention comprising a desired isotypes comprises the steps of isolating a nucleic acid encoding the heavy chain of an IGF-IR antibody and a nucleic acid encoding the light chain of an IGF-IR antibody, obtaining the variable region of the heavy chain, ligating the variable region of the heavy chain with the constant domain of a heavy chain of the desired isotype, expressing the light chain and the ligated heavy chain in a cell, and collecting the IGF-IR antibody with the desired isotype.

Antibody Derivatives

One may use the nucleic acid molecules described above to generate antibody derivatives using techniques and methods known to one of ordinary skill in the art.

Humanized Antibodies

As was discussed above in connection with human antibody generation, there are advantages to producing antibodies with reduced immunogenicity. This can be accomplished to some extent using techniques of humanization and display techniques using appropriate libraries. It will be appreciated that marine antibodies or antibodies from other species can be humanized or primatized using techniques well known in the art. See e.g. Winter and Harris Immunol Today 14:43-46 (1993) and Wright et al. Crit. Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). In a preferred embodiment, the IGF-IR antibody can be humanized by substituting the CH1, CH2, CH3, hinge domains, and/or the framework domain with the corresponding human sequence while maintaining all of the CDRS of the heavy chain, the light chain or both the heavy and light chains.

Mutated Antibodies

In another embodiment, the nucleic acid molecules, vectors, and host cells may be used to make mutated IGF-IR antibodies. The antibodies may be mutated in the variable domains of the heavy and/or light chains to alter a binding property of the antibody. For example, a mutation may be made in one or more of the CDR regions to increase or decrease the K_{d} of the antibody for IGF-IR, to increase or decrease K_{off}, or to alter the binding specificity of the antibody. Techniques in site directed mnutagenesis are well known in the art. See, e.g., Sambrook et al. and Ausubel et al., supra. In a preferred embodiment, mutations are made at an amino acid residue that is known to be changed compared to germline in a variable region of an IGF-IR antibody. In a more preferred embodiment, one or more mutations are made at an amino acid residue that is known to be changed compared to the geimline in a variable region or CDR region of one of the IGF-IR antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In another embodiment, one or more mutations are made at an amino acid residue that is known to be changed compared to the germline in a variable region or CDR region whose amino acid sequence is presented in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or whose nucleic acid sequence is presented in SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38.

In another embodiment, the nucleic acid molecules are mutated in one or more of the framework regions. A mutation may be made in a framework region or constant domain to increase the half-life of the IGF-IR antibody. See, e.g., WO 00/09560, published Feb. 24, 2000, herein incorporated by reference. In one embodiment, there may be one, three, or five point mutations and no more than ten point mutations. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain, and the variable regions in a single mutated antibody. Alternatively, mutations may be made in only one of the framework regions, the variable regions, or the constant domain in a single mutated antibody.

In one embodiment, there are no greater than ten amino acid changes in either the VH or VL regions of the mutated IGF-IR antibody compared to the IGF-IR antibody prior to mutation. In a more preferred embodiment, there are no more than five amino acid changes in either the VH or VL regions of the mutated IGF-IR antibody, more preferably no more than three amino acid changes. In another embodiment, there are no more than fifteen amino acid changes in the constant domains, more preferably, no more than ten amino acid changes, even more preferably, no more than five amino acid changes.

Modified Antibodies

In another embodiment, a fusion antibody or immunoadhesin may be made which comprises all or a portion of an anti-IGF-IR antibody linked to another polypeptide. In a preferred embodiment, only the variable regions of the IGF-IR antibody are linked to the polypeptide. In another preferred embodiment, the VH domain of an IGF-IR antibody are linked to a first polypeptide, while the VL domain of an IGF-IR antibody are linked to a second polypeptide that associates with the first polypeptide in a manner in which the VH and VL domains can interact with one another to form an antibody binding site. In another preferred embodiment, the VH domain is separated from the VL domain by a linker such that the VH and VL domains can interact with one another (see below under Single Chain Antibodies). The VH-linker-VL antibody is then linked to the polypeptide of interest. The fusion antibody is useful to directing a polypeptide to a IGF-IR expressing cell or tissue. The polypeptide may be a therapeutic agent, such as a toxin, growth factor, or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody, (scFv) the VH- and VL-encoding DNA fragments are operatively linked to another fragment encoding a flexible linker, e.g., encoding the amino acid sequence (Gly₄-Ser)₃ (SEQ ID NO:39), such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554). The single chain antibody may be monovalent, if only a single VH and VL are used, bivalent, if two VH and VL are used, or polyvalent, if more than two VH and VL are used.

In another embodiment, other modified antibodies may be prepared using IGF-IR-encoding nucleic acid molecules. For instance, “Kappa bodies” (Ill et al., Protein Eng 10:949-57 (1997)), “Minibodies” (Martin et al., EMBO J 13: 5303 9 (1994)), “Diabodies” (Holliger et al., PNAS USA 90: 6444-6448 (1993)), or “Janusins” (Traunecker et al., EMBO J 10: 3655-3659 (1991) and Traunecker et al. “Janusin: new molecular design for bispecific reagents” Int J Cancer Suppl 7:51-52 (1992)) may be prepared using standard molecular biological techniques following the teachings of the specification.

In another aspect, chimeric and bispecific antibodies can be generated. A chimeric antibody may be made that comprises CDRs and framework regions from different antibodies. In a preferred embodiment, the CDRs of the chimeric antibody comprises all of the CDRs of the variable region of a light chain or heavy chain of an IGF-IR antibody, while the framework regions are derived from one or more different antibodies. In a more preferred embodiment, the CDRs of the chimeric antibody comprise all of the CDRs of the variable regions of the light chain and the heavy chain of a IGF-IR antibody. The framework regions may be from another species and may, in a preferred embodiment, be humanized. Alternatively, the framework regions may be from another human antibody.

A bispecific antibody can be generated that binds specifically to IGF-IR through one binding domain and to a second molecule through a second binding domain. The bispecific antibody can be produced through recombinant molecular biological techniques, or may be physically conjugated together. In addition, a single chain antibody containing more than one VH and VL may be generated that binds specifically to IGF-IR and to another molecule. Such bispecific antibodies can be generated using techniques that are well known for example, in connection with (i) and (ii) see e.g. Fanger et al. Immunol Methods 4: 72-81 (1994) and Wright and Harris, supra. and in connection with (iii) see e.g. Traunecker et al. Int. J. Cancer (Suppl.) 7: 51-52 (1992). In a preferred embodiment, the bispecific antibody binds to IGF-IR and to another molecule expressed at high level on cancer or tumor cells. In a more preferred embodiment, the other molecule is RON, c-Met, erbB2 receptor, VEGF-2 or 3, CD20, or EGF-R.

In another embodiment, the modified antibodies described above are prepared using one or more of the variable regions or one or more CDR regions from one of the antibodies selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5. In another embodiment, the modified antibodies are prepared using one or more of the variable regions or one or more CDR regions whose amino acid sequence is presented in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, or whose nucleic acid sequence is presented in SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38.

Derivatized and Labeled Antibodies

An antibody or antibody portion of the invention can be derivatized or linked to another molecule (e.g., another peptide or protein). In general, the antibodies or portion thereof is derivatized such that the IGF-IR binding is not affected adversely by the derivatization or labeling. Accordingly, the antibodies and antibody portions of the invention are intended to include both intact and modified forms of the human IGF-IR antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase, and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a brown reaction product, which is detectable. An antibody may also be labeled with biotin, and detected through indirect measurement of avidin or streptavidin binding. An antibody may be labeled with a magnetic agent, such as gadolinium. An antibody may also be labeled with a predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

A IGF-IR antibody may also be labeled with a radiolabeled amino acid. The radiolabel may be used for both diagnostic and therapeutic purposes. For instance, the radiolabel may be used to detect IGF-IR-expressing tumors by x-ray or other diagnostic techniques. Further, the radiolabel may be used therapeutically as a toxin for cancerous cells or tumors. Examples of labels for polypeptides include, but are not limited to, the following radioisotopes or radionuclides—³H, ¹⁴C, ¹⁵N, ³⁵S, ⁹⁰Y ⁹⁹Tc ¹¹¹In, ¹²⁵I, and ¹³¹I.

A IGF-IR antibody may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

Pharmaceutical Compositions and Kits

The invention also relates to a pharmaceutical composition for the treatment of a hyperproliferative disorder in a mammal, which comprises a therapeutically effective amount of a compound of the invention and a pharmaceutically acceptable carrier. In one embodiment, said pharmaceutical composition is for the treatment of cancer such as brain, lung, squamous cell, bladder, gastric, pancreatic, breast, head, neck, renal, kidney, ovarian, prostate, colorectal, esophageal, gynecological or thyroid cancer. In another embodiment, said pharmaceutical composition relates to non-cancerous hyperproliferative disorders such as, without limitation, restenosis after angioplasty and psoriasis. In another embodiment, the invention relates to pharmaceutical compositions for the treatment of a mammal that requires activation of IGF-IR, wherein the pharmaceutical composition comprises a therapeutically effective amount of an activating antibody of the invention and a pharmaceutically acceptable carrier. Pharmaceutical compositions comprising activating antibodies may be used to treat animals that lack sufficient IGF-I and IGF-II, or may be used to treat osteoporosis, frailty or disorders in which the mammal secretes too little active growth hormone or is unable to respond to growth hormone. The IGF-IR antibodies of the invention can be incorporated into pharmaceutical compositions suitable for administration to a subject. Typically, the pharmaceutical composition comprises an antibody of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid, and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the IGF-IR antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts, and gelatin.

The antibodies of the present invention can be administered by a variety of methods known in the art, although for many therapeutic applications, the preferred route/mode of administration is intraperitoneal, subcutaneous, intramuscular, intravenous, or infusion. As will be appreciated by the skilled artisan, the route and/or mode of administration will vary depending upon the desired results. In one embodiment, the antibodies of the present inventor can be administered as a single dose or may be administered as multiple doses.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the IGF-IR of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, a IGF-IR antibody of the invention is coformulated with and/or coadministered with one or more additional therapeutic agents, such as a chemotherapeutic agent, an antineoplastic agent, or an anti-tumor agent. For example, a IGF-IR antibody may be coformulated and/or coadministered with one or more additional therapeutic agents. These agents include, without limitation, antibodies that bind other targets (e.g., antibodies that bind one or more growth factors or cytokines, their cell surface receptors or IGF-I and IGF-II), IGF-I and IGF-II binding proteins, antineoplastic agents, chemotherapeutic agents, antitumor agents, antisense oligonucleotides against IGF-IR or IGF-I and IGF-II, peptide analogues that block IGF-IR activation, soluble IGF-IR, and/or one or more chemical agents that inhibit IGF-I and IGF-II production or activity, which are known in the art, e.g., octreotide. For a pharmaceutical composition comprising an activating antibody, the IGF-IR antibody may be formulated with a factor that increases cell proliferation or prevents apoptosis. Such factors include growth factors such as IGF-I and IGF-II, and/or analogues of IGF-I and IGF-II that activate IGF-IR. Such combination therapies may require lower dosages of the IGF-IR antibody as well as the co-administered agents, thus avoiding possible toxicities or complications associated with the various monotherapies. In one embodiment, composition comprises the antibody and one or more additional therapeutic agent.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. Pharmaceutical composition comprising the antibody or comprising a combination therapy comprising the antibody and one or more additional therapeutic agents may be formulated for single or multiple doses. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals. A particularly useful formulation is 5 mg/ml IGF-IR antibody in a buffer of 20 mM sodium citrate, pH 5.5, 140 mM NaCl, and 0.2 mg/ml polysorbate 80.

An exemplary, non-limiting range for a therapeutically or prophylactically effective amount of an antibody or antibody portion of the invention is 0.1-100 mg/kg, more preferably 0.5-50 mg/kg, more preferably 1-20 mg/kg, and even more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. In one embodiment, the therapeutically or prophylactically effective amount of an antibody or antigen-binding portion thereof is administered along with one or more additional therapeutic agents.

Another aspect of the present invention provides kits comprising the IGF-IR antibodies and the pharmaceutical compositions comprising these antibodies. A kit may include, in addition to the antibody or pharmaceutical composition, diagnostic or therapeutic agents. A kit may also include instructions for use in a diagnostic or therapeutic method. In a preferred embodiment, the kit includes the antibody or a pharmaceutical composition thereof and a diagnostic agent that can be used in a method described below. In another preferred embodiment, the kit includes the antibody or a pharmaceutical composition thereof and one or more therapeutic agents, such as an additional antineoplastic agent, anti-tumor agent, or chemotherapeutic agent, which can be used in a method described below.

This invention also relates to pharmaceutical compositions for inhibiting abnormal cell growth in a mammal which comprise an amount of a compound of the invention in combination with an amount of a chemotherapeutic agent, wherein the amounts of the compound, salt, solvate, or prodrug, and of the chemotherapeutic agent are together effective in inhibiting abnormal cell growth. Many chemotherapeutic agents are presently known in the art. In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti angiogenesis agents.

Anti-angiogenic agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with a compound of the invention. Examples of useful COX-II inhibitors include CELEBREX™ (celecoxib), BEXTRA™ (valdecoxib), and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP inhibitors are those that do not demonstrate arthralgia. More preferred, are those that selectively inhibit MMP-2 And/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa bicyclo[3.2.1]octane-3-carboxylic acid hydroxyamide; (2R, 3R) 1-[4-(2-chloro-4 fluoro-benzyloxy)benzenesulfonyl]-3-hydroxy-3-methyl-piperidine-2-carboxylic acid hydroxyamide; 4-[4-(4-fluoro-phenoxy)-benzenesulfonylamino]-tetrahydro pyran-4-carboxylic acid hydroxyamide; 3-[[4-(4-fluoro-phenoxy)benzenesulfonyl](1-hydroxycarbamoyl-cyclobutyl)-amino]-propionic acid; 4[4-(4-chloro-phenoxy) benzenesulfonylamino]-tetrahydro-pyran-4-carboxylic acid hydroxyamide; (R) 3-[4 (4-chloro-phenoxy)-benzenesulfonylamino]tetrahydro-pyran-3-carboxylic acid hydroxyamide; (2R, 3 R) 1-[4-(4-fluoro-2-methyl-benzyloxy)-benzenesulfonyl]-3 hydroxy-3-methyl-piperidine-2-carboxylic acid hydroxyamide; 3-[[4-(4-fluoro phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro pyran-4-yl)-amino]-propionic acid; 3-exo-3-[4-(4-chloro-phenoxy)-benzenesulfonylamino]-8-oxa-icyclo[3.2.1]octane-3-carboxylic acid hydroxyamide; 3-endo-3-[4-(4-fluoro-phenoxy)-benzenesulfonylamino]-8-oxaicyclo[3.2.1]octane-3 carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of said compounds.

A compound of the invention can also be used with signal transduction inhibitors, such as agents that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc.). EGF-R inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22 Mab (ImClone Systems Incorporated), ABX-EGF (Abgenix/Cell Genesys), EMD-7200 (Merck KgaA), EMD-5590 (Merck KgaA), MDX-447/H-477 (Medarex Inc. and Merck KgaA), and the compounds ZD 1834, ZD-1838 and ZD-1839 (AstraZeneca), PKI-166 (Novartis), PKI-166/CGP 75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co.), VRCTC 310 (Ventech Research), EGF fusion toxin (Seragen Inc.), DAB-389 (Seragen/Ligand), ZM-252808 (Imperial Cancer Research Fund), RG-50864 (INSEAM), LFM-A12 (Parker Hughes Cancer Center), WHI-P97 (Parker Hughes Cancer Center), GW-282974 (Glaxo), KT-8391 (Kyowa Hakko) and EGF-R Vaccine (York Medical/Centro de Immunologia Molecular (CIM)). These and other EGF-R inhibiting agents can be used in the present invention.

VEGF inhibitors, for example SU-11248 (Sugen Inc.), SH-268 (Schering), and NX-1838 (NeXstar) can also be combined with the compound of the present invention. VEGF inhibitors are described in, for example in WO 99/24440 (published May 20, 1999), PCT International Application PCT/1B99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc.); anti-VEGF monoclonal antibody of Genentech, Inc.; and angiozyme, a synthetic ribozyme from Ribozyme and Chiron. These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc.) and 2B-I (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5, 877,305 (issued Mar. 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compound of the present invention in accordance with the present invention.

Another component of the combination of the present invention is a cycloxygenase-2 selective inhibitor. The terms “cyclooxygenase-2 selective inhibitor”, or “Cox-2 selective inhibitor”, which can be used interchangeably herein, embrace compounds, which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the tern “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

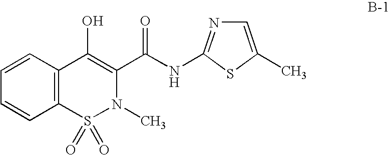

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

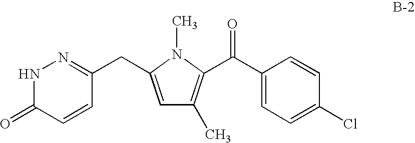

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or. dihydronaphthalenes. Benzopyrans that can serve as a cyclooxygenase-2 selective inhibitor of the present invention include substituted benzopyran derivatives that are described in U.S. Pat. No. 6,271,253. Other benzopyran Cox-2 selective inhibitors useful in the practice of the present invention are described in U.S. Pat. Nos. 6,034,256 and 6,077,850.

In a further preferred embodiment of the invention the cyclooxygenase inhibitor can be selected from the class of tricyclic cyclooxygenase-2 selective inhibitors represented by the general structure of formula I:

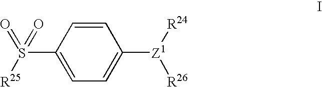

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkyiamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula I is selected from the group of compounds, illustrated in Table 3, which includes celecoxib (B-3), valdecoxib (B-4), deracoxib (B-5), rofecoxib (B-6), etoricoxib (MK-663; B-7), JTE-522 (B-8), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 1.69590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 3

| Compound | |
|---|---|
| Number | Structural Formula |
| B-3 | 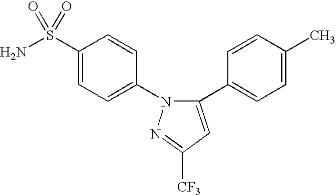 |
| B-4 | 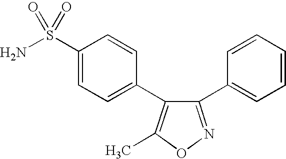 |
| B-5 | 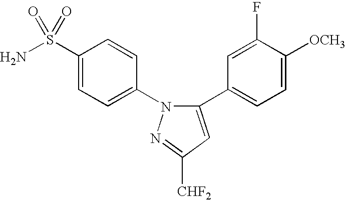 |
| B-6 | 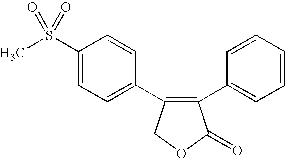 |
| B-7 | 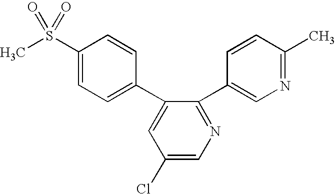 |
| B-8 | 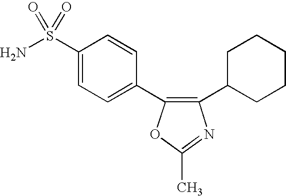 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-9, which is a therapeutically effective prodrug of the tricyclic cyclooxygenase-2 selective inhibitor valdecoxib, B-4, (See, e.g., U.S. Pat. No. 5,633,272), may be advantageously employed as a source of a cyclooxygenase inhibitor.

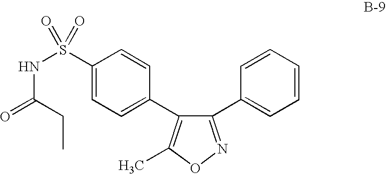

A preferred form of parecoxib is sodium parecoxib.

In another embodiment of the invention, the compound ABT-963 having the formula B-10 that has been previously described in International Publication number WO 00/24719, is another tricyclic cyclooxygenase-2 selective inhibitor, which may be advantageously employed.

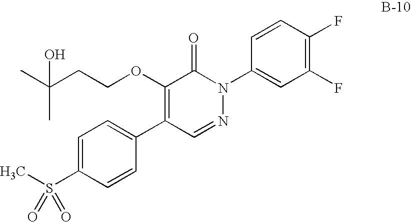

In a further embodiment of the invention, the cyclooxygenase inhibitor can be selected from the class of phenylacetic acid derivative cyclooxygenase-2 selective inhibitors described in WO 99/11605 WO 02/20090 is a compound that is referred to as COX-189 (also termed lumiracoxib), having CAS Reg. No. 220991-20-8.

Compounds that have a structure similar can serve as the Cox-2 selective inhibitor of the present invention, are described in U.S. Pat. Nos. 6,310,099, 6,291,523, and 5,958,978.

Further information on the applications of the Cox-2 selective inhibitor N-(2-cyclohexyloxynitrophenyl) methane sulfonamide (NS-398, CAS RN 123653-11-2), having a structure as shown in formula B-11, have been described by, for example, Yoshimi, N. et al., in Japanese J. Cancer Res., 90(4):406-412 (1999); Falgueyret, J.-P. et al., in Science Spectra, available at: http://www.gbhap.com/Science-_Spectra/20-1-article.htm (06/06/2001); and Iwata, K. et al., in Jpn. J. Pharmacol., 75(2):191-194 (1997).

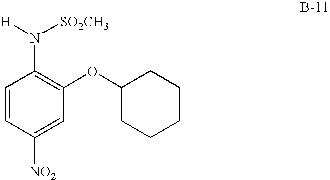

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 04, 2001, where it was reported that S-33516 is a tetrahydroisoinde derivative that has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covalently attached to one or more linkers, as described in U.S. Pat. No. 6,395,724. Compounds that may act as cyclooxygenase-2 inhibitors include conjugated linoleic acid that is described in U.S. Pat. No. 6,077,868. Materials that can serve as a cyclooxygenase-2 selective inhibitor of the present invention include heterocyclic aromatic oxazole compounds that are described in U.S. Pat. Nos. 5,994,381 and 6,362,209. Cox-2 selective inhibitors that are useful in the subject method and compositions can include compounds that are described in U.S. Pat. Nos. 6,080,876 and 6,133,292. Materials that can serve as cyclooxygenase-2 selective inhibitors include pyridines that are described in U.S. Pat. Nos. 6, 369,275, 6,127,545, 6,130,334, 6,204,387, 6,071,936, 6,001,843 and 6,040,450. Materials that can serve as the cyclooxygenase-2 selective inhibitor of the present invention include diarylbenzopyran derivatives that are described in U.S. Pat. No. 6,340,694. Materials that can serve as the cyclooxygenase-2 selective inhibitor of the present invention include 1-(4-sulfamylaryl)-3-substituted-5-aryl-2-pyrazolines are described in U.S. Pat. No. 6,376,519.

Materials that can serve as the cyclooxygenase-2 selective inhibitor of the present invention include heterocycles that are described in U.S. Pat. No. 6,153,787. Materials that can serve as the cyclooxygenase-2 selective inhibitor of the present invention include 2,3,5-trisubstituted pyridines that are described in U.S. Pat. No. 6,046,217. Materials that can serve as the cyclooxygenase-2 selective inhibitor of the present invention include diaryl bicyclic heterocycles that are described in U.S. Pat. No. 6,329,421. Compounds that may act as cyclooxygenase-2 inhibitors include salts of 5-amino or a substituted amino 1,2,3-triazole compounds that are described in U.S. Pat. No. 6,239,137.

Materials that can serve as a cyclooxygenase-2 selective inhibitor of the present invention include pyrazole derivatives that are described in U.S. Pat. No. 6,136,831. Materials that can serve as a cyclooxygenase-2 selective inhibitor of the present invention include substituted derivatives of benzosulphonamides that are described in U.S. Pat. No. 6,297,282. Materials that can serve as a cyclooxygenase-2 selective inhibitor of the present invention include bicycliccarbonyl indole compounds that are described in U.S. Pat. No. 6,303,628. Materials that can serve as a cyclooxygenase-2 selective inhibitor of the present invention include benzimidazole compounds that are described in U.S. Pat. No. 6,310,079. Materials that can serve as a cyclooxygenase-2 selective inhibitor of the present invention include indole compounds that are described in U.S. Pat. No. 6,300,363. Materials that can serve as a cyclooxygenase-2 selective inhibitor of the present invention include aryl phenylhydrazides that are described in U.S. Pat. No. 6,077,869. Materials that can serve as a cyclooxygenase-2 selective inhibitor of the present invention include 2-aryloxy, 4-aryl furan-2-ones that are described in U.S. Pat. No. 6,140,515. Materials that can serve as a cyclooxygenase-2 selective inhibitor of the present invention include bisaryl compounds that are described in U.S. Pat. No. 5,994,379. Materials that can serve as a cyclooxygenase-2 selective inhibitor of the present invention include 1,5-diarylpyrazoles that are described in U.S. Pat. No. 6,028,202. Materials that can serve as a cyclooxygenase-2 selective inhibitor of the present invention include 2-substituted imidazoles that are described in U.S. Pat. No. 6,040,320. Materials that can serve as a cyclooxygenase-2 selective inhibitor of the present invention include 1,3- and 2,3-diarylcycloalkano and cycloalkeno pyrazoles that are described in U.S. Pat. No. 6,083,969. Materials that can serve as a cyclooxygenase-2 selective inhibitor of the present invention include esters derived from indolealkanols and novel amides derived from indolealkylamides that are described in U.S. Pat. No. 6,306,890. Materials that can serve as a cyclooxygenase-2 selective inhibitor of the present invention include pyridazinone compounds that are described in U.S. Pat. No. 6,307,047. Materials that can serve as a cyclooxygenase-2 selective inhibitor of the present invention include benzosulphonamide derivatives that are described in U.S. Pat. No. 6,004,948. Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

Anti-survival agents include IGF-IR antibodies and anti-integrin agents, such as anti-integrin antibodies.

Diagnostic Methods of Use

The IGF-IR antibodies may be used to detect IGF-IR in a biological sample if in vitro or in vivo. The IGF-IR antibodies may be used in a conventional immunoassay, including, without limitation, an ELISA, an RIA, FACS, tissue immunohistochemistry, Western blot, or immunoprecipitation. The IGF-IR antibodies of the invention may be used to detect IGF-IR from humans. In another embodiment, the IGF-IR antibodies may be used to detect IGF-IR from Old World primates such as cynomolgus and rhesus-monkeys, chimpanzees and apes.

The invention provides a method for detecting IGF-IR in a biological sample comprising contacting a biological sample with an IGF-IR antibody of the invention and detecting the bound antibody bound to IGF-IR, to detect the IGF-IR in the biological sample. In one embodiment, the IGF-IR antibody is directly labeled with a detectable label. In another embodiment, the IGF-IR antibody (the first antibody) is unlabeled and a second antibody or other molecule that can bind the IGF-IR antibody and is labeled. As is well known to one of skill in the art, a second antibody is chosen that is able to specifically bind the specific species and class of the first antibody. For example, if the IGF-IR antibody is a human IgG, then the secondary antibody may be an anti-human-IgG. Other molecules that can bind to many antibodies include, without limitation, Protein A and Protein G, both of which are available commercially, e.g., Amersham Pharmacia Biotech. Suitable labels for the antibody or secondary detection antibodies have been disclosed supra, and include various enzymes, prosthetic groups, fluorescent materials, luminescent materials, magnetic agents-and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; an example of a magnetic agent includes gadolinium; and examples of suitable radioactive material include ¹²⁵I, ¹³¹I, ³⁵S or ³H.

In an alternative embodiment, IGF-IR can be assayed in a biological sample by a competition immunoassay utilizing IGF-IR standards labeled with a delectable substance and an unlabeled IGF-IR antibody. In this assay, the biological sample, the labeled IGF-IR standards, and the IGF-IR antibody are combined and the amount of labeled IGF-IR standard bound to the unlabeled antibody is determined. The amount of IGF-IR in the biological sample is inversely proportional to the amount of labeled IGF-IR standard bound to the IGF-IR antibody.

One may use the immunoassays disclosed above for a number of purposes. In one embodiment, the IGF-IR antibodies may be used to detect IGF-IR present in cells in cell culture. In a preferred embodiment, the IGF-IR antibodies may be used to determine the level of tyrosine phosphorylation, tyrosine autophosphorylation of IGF-IR, and/or the amount of IGF-IR on the cell surface after treatment of the cells with various compounds. This method can be used to test compounds that may be used to activate or inhibit IGF-IR, or result in a redistribution of IGF-IR on the cell surface or intracellularly. In this method, one sample of cells is treated with a test compound for a period of time while another sample is left untreated. If tyrosine autophosphorylation is to be measured, the cells are lysed and tyrosine phosphorylation of the IGF-IR is measured using an immunoassay described above or as described in Example III, which uses an ELISA. If the total level of IGF-IR is to be measured, the cells are lysed and the total IGF-IR level is measured using one of the immunoassays described above. The level of cell-surface IGF-IR may be determined using antibodies of the invention staining tissue culture cells following fixation of the cells. Standard practices of those skilled in the art allow fluorescence-activated cell sorting (FACS) to be used with a secondary detection antibody to determine the amount of binding of the primary (IGF-IR) antibody to the cell surface. Cells may also be permeablized with detergents or toxins to allow the penetration of normally impermeant antibodies to now label intracellular sites where IGF-IR is localized.

A preferred immunoassay for determining IGF-IR tyrosine phosphorylation or for measuring total IGF-IR levels is an ELISA or Western blot. If only the cell surface level of IGF-IR is to be measured, the cells are not lysed, and the cell surface levels of IGF-IR are measured using one of the immunoassays described above (e.g., FACS). A preferred immunoassay for determining cell surface levels of IGF-IR includes the steps of labeling exclusively the cell surface proteins with a detectable label, such as biotin or ¹²⁵I, immunoprecipitating a detergent-soluble fraction of the cells containing integral membrane proteins with a IGF-IR antibody, and then detecting the fraction of total IGF-IR containing the detectable label. Another preferred immunoassay for determining the localization of IGF-IR, e.g., cell surface levels is by using immunofluorescence or immunohistochemistry. Methods such as ELISA, RIA, Western blot, immunohistochemistry, cell surface labeling of integral membrane proteins and immunoprecipitation are well known in the art. See, e.g., Harlow and Lane, supra. In addition, the immunoassays may be scaled up for high throughput screening in order to test a large number of compounds for either activation or inhibition of IGF-IR.

The IGF-IR antibodies of the invention may also be used to determine the levels of IGF-IR in a tissue or in cells derived from the tissue. In a preferred embodiment, the tissue is a diseased tissue. In a more preferred embodiment, the tissue is a tumor or a biopsy thereof. In a preferred embodiment of the method, a tissue or a biopsy thereof is excised from a patient. The tissue or biopsy is then used in an immunoassay to determine, e.g., IGF-IR levels, cell surface levels of IGF-IR, levels of tyrosine phosphorylation of IGF-IR, or localization of IGF-IR by the methods discussed above. The method can be used to determine if a tumor expresses IGF-IR at a high level.

The above-described diagnostic method can be used to determine whether a tumor expresses high levels of IGF-IR, which may he indicative that the tumor will respond well to treatment with IGF-IR antibody. The diagnostic method may also be used to determine whether a tumor is potentially cancerous, if it expresses high levels of IGF-IR, or benign, if it expresses low levels of IGF-IR. Further, the diagnostic method may also be used to determine whether treatment with IGF-IR antibody (see below) is causing a tumor to express lower levels of IGF-IR and/or to express lower levels of tyrosine autophosphorylation, and thus can be used to determine whether the treatment is successful. In general, a method to determine whether an IGF-IR antibody decreases tyrosine phosphorylation comprises the steps of measuring the level of tyrosine phosphorylation in a cell or tissue of interest, incubating the cell or tissue with an IGF-IR antibody or antigen-binding portion thereof, then re-measuring the level of tyrosine phosphorylation in the cell or tissue. The tyrosine phosphorylation of IGF-IR or of another protein(s) may be measured. The diagnostic method may also be used to determine whether a tissue or cell is not expressing high enough levels of IGF-IR or high enough levels of activated IGF-IR, which may be the case for individuals with dwarfism, osteoporosis, or diabetes. A diagnosis that levels of IGF-IR or active IGF-IR are too low could be used for treatment with activating IGF-IR antibodies, IGF-I and IGF-II or other therapeutic agents for increasing IGF-IR levels or activity.

The antibodies of the present invention may also be used in vivo to localize tissues and organs that express IGF-IR. In a preferred embodiment, the IGF-IR antibodies can be used to localize IGF-IR expressing tumors. The advantage of the IGF-IR antibodies of the present invention is that they will not generate an immune response upon administration. The method comprises the steps of administering an IGF-IR antibody or a pharmaceutical composition thereof to a patient in need of such a diagnostic test and subjecting the patient to imaging analysis determine the location of the IGF-IR expressing tissues. Imaging analysis is well known in the medical art, and includes, without limitation, x-ray analysis, magnetic resonance imaging (MRI), or computed tomography (CE). In another embodiment of the method, a biopsy is obtained from the patient to determine whether the tissue of interest expresses IGF-IR rather than subjecting the patient to imaging analysis. In a preferred embodiment, the IGF-IR antibodies may be labeled with a detectable agent that can be imaged in a patient. For example, the antibody may be labeled with a contrast agent, such as barium, which can be used for x-ray analysis, or a magnetic contrast agent, such as a gadolinium chelate, which can be used f6r MRI or CE. Other labeling agents include, without limitation, radioisotopes, such as ⁹⁹Tc. In another embodiment, the IGF-IR antibody will be unlabeled and will be imaged by administering a second antibody or other molecule that is detectable and that can bind the IGF-IR antibody.

Therapeutic Methods of Use

In another embodiment, the invention provides a method for inhibiting IGF-IR activity by administering a IGF-IR antibody to a patient in need thereof. Any of the types of antibodies described herein may be used therapeutically. In a preferred embodiment, the IGF-IR antibody is a human, chimeric, or humanized antibody. In another preferred embodiment, the IGF-IR is human and the patient is a human patient. Alternatively, the patient may be a mammal that expresses a IGF-IR that the IGF-IR antibody cross-reacts with. The antibody may be administered to a nonhuman mammal expressing a IGF-IR with which the antibody cross-reacts (i.e. a primate, or a cynomolgus or rhesus monkey) for veterinary purposes or as an animal model of human disease. Such animal models may be useful for evaluating the therapeutic efficacy of antibodies of this invention.

As used herein, the term “a disorder in which IGF-IR activity is detrimental” is intended to include diseases and other disorders in which the presence of high levels of IGF-IR in a subject suffering from the disorder has been shown to be or is suspected of being either responsible for the pathophysiology of the disorder or a factor that contributes to a worsening of the disorder. Accordingly, a disorder in which high levels of IGF-IR activity is detrimental is a disorder in which inhibition of IGF-IR activity is expected to alleviate the symptoms and/or progression of the disorder. Such disorders may be evidenced, for example, by an increase in the levels of IGF-IR on the cell surface or in increased tyrosine autophosphorylation of IGF-IR in the affected cells or tissues of a subject suffering from the disorder. The increase in IGF-IR levels may be detected, for example, using a IGF-IR antibody as described above.

In a preferred embodiment, a IGF-IR antibody may be administered to a patient who has a IGF-IR-expressing tumor. A tumor may be a solid tumor or may be a non-solid tumor, such as a lymphoma. In a more preferred embodiment, an anti-IGF-antibody may be administered to a patient who has a IGF-IR-expressing tumor that is cancerous. In an even more preferred embodiment, the IGF-IR antibody is administered to a patient who has a tumor of the lung, breast, prostate, or colon. In a highly preferred embodiment, the method causes the tumor not to increase in weight or volume or to decrease in weight or volume. In another embodiment, the method causes the IGF-IR on the tumor to be internalized. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, or comprises a heavy chain, light chain or antigen-binding region thereof.

In another preferred embodiment, a IGF-IR antibody may be administered to a patient who expresses inappropriately high levels of IGF-I and IGF-II. It is known in the art that high level expression of IGF-I and IGF-II can lead to a variety of common cancers. In a more preferred embodiment, the IGF-IR antibody is administered to a patient with prostate cancer, glioma, or fibrosarcoma. In an even more preferred embodiment, the method causes the cancer to stop proliferating abnormally, or not to increase in weight or volume or to decrease in weight or volume.

In one embodiment, said method relates to the treatment of cancer such as brain, squamous cell, bladder, gastric, pancreatic, breast, head, neck, esophageal, prostate, colorectal, lung, renal, kidney, ovarian, gynecological or thyroid cancer. Patients that can be treated with a compounds of the invention according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, Wilm's tumor, mesothelioma, neuroblastoma, Ewing's sarcoma, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The antibody may be administered once, but more preferably is administered multiple times. The antibody may be administered from three times daily to once every six months. The administering may be on a schedule such as three times daily, twice daily, once daily, once every two days, once every three days, once weekly, once every two weeks, once every month, once every two months, once every three months and once every six months. The antibody may be administered via an oral, mucosal, buccal, intranasal, inhalable, intravenous, subcutaneous, intramuscular, parenteral, intratumor, or topical route. The antibody may be administered at a site distant from the site of the tumor. The antibody may also be administered continuously via a minipump. The antibody may be administered once, at least twice or for at least the period of time until the condition is treated, palliated, or cured. The antibody generally will be administered for as long as the tumor is present provided that the antibody causes the tumor or cancer to stop growing or to decrease in weight or volume. The antibody will generally be administered as part of a pharmaceutical composition as described supra. The dosage of antibody will generally be in the range of 0.1-100 mg/kg, more preferably 0.5-50 mg/kg, more preferably 1-20 mg/kg, and even more preferably 1-10 mg/kg. The serum concentration of the antibody may be measured by any method known in the art. The antibody may also be administered prophylactically in order to prevent a cancer or tumor from occurring. This may be especially useful in patients that have a “high normal” level of IGF-I and IGF-II because these patients have been shown to have a higher risk of developing common cancers. See Rosen et al. supra.

In another aspect, the IGF-IR antibody may be co-administered with other therapeutic agents, such as antineoplastic drugs or molecules, to a patient who has a hyperproliferative disorder, such as cancer or a tumor. In one aspect, the invention relates to a method for the treatment of the hyperproliferative disorder in a mammal comprising administering to said mammal a therapeutically effective amount of a compound of the invention in combination with an anti-tumor agent selected from the group consisting of, but not limited to, mitotic inhibitors, alkylating agents, anti-metabolites, intercalating agents, growth factor inhibitors, cell cycle inhibitors, enzymes, topoisomerase inhibitors, biological response modifiers, anti-hormones, kinase inhibitors, matrix metalloprotease inhibitors, genetic therapeutics and anti androgens. In a more preferred embodiment, the antibody may be administered with an antineoplastic agent, such as Adriamycin or taxol. In another preferred embodiment, the antibody or combination therapy is administered along with radiotherapy, chemotherapy, photodynamic therapy, surgery, or other immunotherapy. In yet another preferred embodiment, the antibody will be administered with another antibody. For example, the IGF-IR antibody may be administered with an antibody or other agent that is known to inhibit tumor or cancer cell proliferation, e.g., an antibody or agent that inhibits erbB2 receptor, EGF-R, CD20, or VEGF.

Co-administration of the antibody with an additional therapeutic agent (combination therapy) encompasses administering a pharmaceutical composition comprising the IGF-IR antibody and the additional therapeutic agent and administering two or more separate pharmaceutical compositions, one comprising the IGF-IR antibody and the other(s) comprising the additional therapeutic agent(s). Further, although co-administration or combination therapy generally means that the antibody and additional therapeutic agents are administered at the same time as one another, it also encompasses instances in which the antibody and additional therapeutic agents are administered at different times. For instance, the antibody may be administered once every three days, while the additional therapeutic agent is administered once daily. Alternatively, the antibody may be administered prior to or subsequent to treatment of the disorder with the additional therapeutic agent. Similarly, administration of the IGF-IR antibody may be administered prior to or subsequent to other therapy, such as radiotherapy, chemotherapy, photodynamic therapy, surgery, or other immunotherapy

The antibody and one or more additional therapeutic agents (the combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously via a minipump. The combination therapy may be administered via an oral, mucosal, buccal, intranasal, inhalable, intravenous, subcutaneous, intramuscular, parenteral, intratumor or topical route. The combination therapy may be administered at a site distant from the site of the tumor. The combination therapy generally will be administered for as long as the tumor is present provided that the antibody causes the tumor or cancer to stop growing or to decrease in weight or volume.

In a still further embodiment, the IGF-IR antibody is labeled with a radiolabel, an immunotoxin, or a toxin, or is a fusion protein comprising a toxic peptide. The IGF-IR antibody or IGF-IR antibody fusion protein directs the radiolabel, immunotoxin, toxin, or toxic peptide to the IGF-IR-expressing tumor or cancer cell. In a preferred embodiment, the radiolabel, immunotoxin, toxin, or toxic peptide is internalized after the IGF-IR antibody binds to the IGF-IR on the surface of the tumor or cancer cell.

In another aspect, the IGF-IR antibody may be used therapeutically to induce apoptosis of specific cells in a patient in need thereof. In many cases, the cells targeted for apoptosis are cancerous or tumor cells. Thus, in a preferred embodiment, the invention provides a method of inducing apoptosis by administering a therapeutically effective amount of a IGF-IR antibody to a patient in need thereof. In a preferred embodiment, the antibody is selected from PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, or comprises a heavy chain, light chain, or antigen-binding region thereof.

In another aspect, the IGF-IR antibody may be used to treat noncancerous states in which high levels of IGF-I and IGF-II and/or IGF-IR have been associated with the noncancerous state or disease. In one embodiment, the method comprises the step of administering a IGF-IR antibody to a patient who has a noncancerous pathological state caused or exacerbated by high levels of IGF-I and IGF-II and/or IGF-IR levels or activity. In a preferred embodiment, the noncancerous pathological state is psoriasis, atherosclerosis, smooth muscle restenosis of blood vessels or inappropriate microvascular proliferation, such as that found as a complication of diabetes, especially of the eye. In a more preferred embodiment, the IGF-IR antibody slows the progress of the noncancerous pathological state. In a more preferred embodiment, the IGF-IR antibody stops or reverses, at least in part, the noncancerous pathological state.

The antibodies of the present would also be useful in the treatment or prevention of ophthalmic diseases, for example glaucoma, retinitis, retinopathies (e.g., diabetic retinopathy), uveitis, ocular photophobia, macular degeneration (e.g., age related macular degeneration, wet-type macular degeneration, and dry-type macular degeneration) and of inflammation and pain associated with acute injury to the eye tissue. The compounds would be further useful in treatment or prevention of postsurgical ophthalmic pain and inflammation.

In another aspect, the invention provides a method of administering an activating IGF-IR antibody to a patient in need thereof. In one embodiment, the activating antibody or pharmaceutical composition is administered to a patient in need thereof in an amount effective to increase IGF-IR activity. In a more preferred embodiment, the activating antibody is able to restore normal IGF-IR activity. In another preferred embodiment, the activating antibody may be administered to a patient who has small stature, neuropathy, a decrease in muscle mass or osteoporosis. In another preferred embodiment, the activating antibody may be administered with one or more other factors that increase cell proliferation, prevent apoptosis, or increase IGF-IR activity. Such factors include growth factors such as IGF-I and IGF-II, and/or analogues of IGF-I and IGF-II that activate IGF-IR.

Gene Therapy

The nucleic acid molecules of the instant invention may be administered to a patient in need thereof via gene therapy. The therapy may be either in vivo or ex viva. In a preferred embodiment, nucleic acid molecules encoding both a heavy chain and a light chain are administered to a patient. In a more preferred embodiment, the nucleic acid molecules are administered such that they are stably integrated into the chromosome of B cells because these cells are specialized for producing antibodies. In a preferred embodiment, precursor B cells are transfected or infected ex vivo and retransplanted into a patient in need thereof. In another embodiment, precursor B cells or other cells are infected in vivo using a virus known to infect the cell type of interest. Typical vectors used for gene therapy include liposomes, plasmids, or viral vectors, such as retroviruses, adenoviruses, and adeno associated viruses. After infection either in viva or ex vivo, levels of antibody expression may be monitored by taking a sample from the treated patient and using any immunoassay known in the art and discussed herein.

In a preferred embodiment, the gene therapy method comprises the steps of administering an effective amount of an isolated nucleic acid molecule encoding the heavy chain or the antigen-binding portion thereof of the human antibody or portion thereof and expressing the nucleic acid molecule. In another embodiment, the gene therapy method comprises the steps of administering an effective amount of an isolated nucleic acid molecule encoding the light chain or the antigen-binding portion thereof of the human antibody or portion thereof and expressing the nucleic acid molecule. In a more preferred method, the gene therapy method comprises the steps of administering an effective amount of an isolated nucleic acid molecule encoding the heavy chain or the antigen binding portion thereof of the human antibody or portion thereof and an effective amount of an isolated nucleic acid molecule encoding the light chain or the antigen-binding portion thereof of the human antibody or portion thereof and expressing the nucleic acid molecules. The gene therapy method may also comprise the step of administering another anti cancer agent, such as taxol, tamoxifen, 5-FU, Adriamycin or CP-358,774.

In order that this invention may be better understood, the following examples are set forth. These examples are for purposes of illustration only and are not to be construed as limiting the scope of the invention in any manner.

### EXAMPLES

### Example 1

### Selection of IGF-IR Binding ScFvs

An scFv phagemid library, which is an expanded version of the 1.38×10¹⁰ library described by Vaughan et al. (Nature Biotech. (1996) 14: 309-314) was used to select antibodies specific for human IGF1R. Three selection methodologies were employed; panning selection, soluble selection, and selection on the surface of a transfected cell-line.

For the panning method, soluble IGF1R extracellular domain (ECD) fusion protein (at 10 μg/ml in phosphate buffered saline (PBS)) or control fusion protein (at 10 μg/ml in PBS) was coated onto the wells of a microtiter plate overnight at 4° C. In addition, soluble IGF1R ECD (at 5 μg/ml in PBS) was covalently coupled to the wells of a microtiter plate overnight at 4° C. In both cases, the wells were washed in PBS and blocked for 1 hour at 37° C. in MPBS (3% milk powder in PBS). Purified phage (10¹² transducing units (tu)) were blocked for 1 hour in a final volume of 100 μl of 3% MPBS. For the IGFIR ECD fusion protein selections, blocked phage were added to blocked control fusion protein wells and incubated for 1 hour. The blocked and deselected phage were then transferred to the blocked wells that were coated with the IGF1R ECD fusion protein and incubated for an additional hour. For the selections with covalently coupled IGF1R ECD, blocked phage were added directly to the blocked wells that contained coupled IGF1R ECD and incubated for 1 hour. In both cases, wells were washed 5 times with PBST (PBS containing 0.1% v/v Tween 20), then 5 times with PBS. Bound phage particles were eluted and used to infect 10 ml of exponentially growing E. coli TG 1. Infected cells were grown in 2TY broth for 1 hour at 37° C., then spread onto 2TYAG plates and incubated overnight at 30° C. Colonies were scraped off the plates into 10 ml 2TY broth and 15% glycerol solution added for storage at −70° C.

Glycerol stock cultures from the first round panning selection were superinfected with helper phage and rescued to give scFv antibody-expressing phage particles for the second round of panning. A total of three rounds of panning were carried out in this way for isolation of antibody-expressing phage particles specific for human IGF1R.

For the soluble selection method, biotinylated human IGF1R ECD fusion protein at a final concentration of 50 nM was used with scFv phagemid library, as described above. Purified scFv phage (10¹² tu) in 1 ml 3% MPBS were blocked for 30 minutes, then biotinylated antigen was added and incubated at room temperature for 1 hour. Phage/antigen was added to 50 μl of Dynal M280 Streptavidin magnetic beads that had been blocked for 1 hour at 37° C. in 1 ml of 3% MPBS and incubated for a further 15 minutes at room temperature. Beads were captured using a magnetic rack and washed 5× in 1 ml of 3% MPBS/0.1% (v/v) Tween 20 followed by 2 washes in PBS. After the last PBS wash, beads were resuspended in 100 μl PBS and used to infect 5 ml of exponentially growing E. coli TG-1 cells. Infected cells were incubated for 1 hour at 37° C. (30 minutes stationary, 30 minutes shaking at 250 rpm), then spread on 2TYAG plates and incubated overnight at 30° C. Output colonies were scraped off the plates and phage rescued as described above. Two further rounds of soluble selection were performed as described above.

For cell-surface selections, NIH3T3 cells transfected with human IGF1R and untransfected control NIH3T3 cells were seeded at 4×10⁵ cells per well and allowed to reach confluence. Purified phage (10¹² transducing units (tu)) were blocked for 1 hour in a final volume of 500 μl of 4% milk powder in culture media (DMEM/FCS). Blocked phage were added to blocked, untransfected control cells and incubated for 1 hour. The blocked and deselected phage were then transferred to blocked NIH3T3 cells transfected with human IGF1R and incubated at room temperature for 1 hour. Wells were washed 2 times with PBST (PBS containing 0.1% v/v Tween 20), then 2 times with PBS. Bound phage particles were eluted and used to infect 10 ml of exponentially growing E. coli TG 1. Infected cells were grown in 2TY broth for I hour at 37° C., then spread onto 2TYAG plates and incubated overnight at 30° C. Colonies were scraped off the plates into 10 ml 2TY broth and 15% glycerol solution added for storage at −70° C.

### Example 2

### IGF-IR Antibody Expression and Purification

Clones were converted into the IgG format as described below. Reformatting involves the subcloning of the VH domain from the scFv into a vector containing the human heavy chain constant domains, and regulatory elements for the appropriate expression in mammalian cells. Similarly, the VL domain is subcloned into an expression vector containing the human light chain constant domain (lambda or kappa class) along with the appropriate regulatory elements

The nucleic acid sequence encoding the appropriate domain from the scFv clone was amplified, followed by restriction enzyme digestion and ligation into the appropriate expression vector. Heavy Chain (IgG1 constant domain) were cloned into pEU1, Light Chain (lambda class) were cloned into pEU4, and Light Chain (kappa class) were cloned into pEU3 (Persic, L. et al., Gene 187:9-18 (1997))

Site Directed Mutagenesis

Prior to reformatting, it was observed that several scFvs (including PGIA-03-A11) contained an internal BstEII restriction site within the VH domain that would interfere with cloning of the VH into the IgG1 heavy chain vector. The internal restriction site was removed by Quikchange™ (Invitrogen) site-directed mutagenesis using the method as described in the kit. Oligos were designed to remove the restriction site but maintaining the same amino acid sequence. Sequencing was carried out to ensure that the site had been mutated correctly. Mutagenesis primers are shown in Table 4.

TABLE 4

| Oligo | | |
|---|---|---|
| name | nucleotide sequence (5′-3′) | Oligo function |
| 7A2 MF | GTCCTTCCAAGGCCAGGTCACGATCTC | quick change |
| | SEQ ID NO:40 | 7A2VH stop codon |
| | | to Q forward |
| | | primer |
| 7A2 MR | GAGATCGTGACCTGGCCTTGGAAGGAC | quick change |
| | SEQ ID NO:41 | 7A2VH stop codon |
| | | to Q reverse |
| | | primer |
| 7A4 MF | CCAAGCTGACCGTCCTAGGTGAG | quick change |
| | SEQ ID NO:42 | 7A4VL S/A |
| | | forward primer |
| 7A4 MR | CTCACCTAGGACGGTCAGCTTGG | quick change |
| | SEQ ID NO:43 | 7A4VL S/A |
| | | reverse primer |
| 8A1-MF | CGTCCTTCCAAGGCCAAGTCACCATCT | Removes BstEII |
| | CAGTCG SEQ ID NO:44 | site from 8A1 |
| | | VH, forward |
| | | primer |
| 8A1-MR | CGACTGAGATGGTGACTTGGCCTTGGA | Removes BstEII |
| | AGGACG SEQ ID NO:45 | site from 8A1 |
| | | VH, reverse |
| | | primer |

VH/VL Cloning PCR

Once all sequences were checked for the absence of restriction sites, the nucleic acid sequence encoding the VH and VL domains were amplified in separate PCR reactions.

100 ul PCR reactions were set up for each VH and VL domain using 50 ul 2×PCR master mix, 5 ul forward primer (@10 uM), 5 ul reverse primer (@10 uM), and 40 ul water. Primers were allocated according to the scFv sequence, and are shown in Table 5

TABLE 5

| scFv | VH Forward | VH reverse | VL forward | VL reverse |
|---|---|---|---|---|
| Clone | primer | primer | primer | primer |
| 7A2 | 7A2VHF | 7A2VHR | AF32 | AF23 |
| 7A4 | 7A4VHF | 7A4VHR | 7A4VLF | 7A4VLR |
| 7A5 | 7A5VHF | 7A5VHR | AF32 | AF23 |
| 7A6 | 7A6VHF | 7A6VHR | AF32 | AF23 |
| 8A1 | 8A1VHF | 8A1VHR | 8A1VLF | 8A1VLR |
| 9A1 | 9A1VHF | 9A1VHR | 9A1VLF | 9A1VLR |
| 9A2 | 9A2VHF | 9A2VHR | 9A2VLF | 9A2VLR |
| 11A1 | 11A1VHF | 11A1VHR | 11A1VLF | 11A1VLR |
| 11A2 | 11A2VHF | 11A2VHR | 11A2VLF | 11A2VLR |
| 11A3 | 11A3VHF | 11A3VHR | 11A3VLF | 11A3VLR |
| 11A4 | 11A4VHF | 11A4VHR | 11A4VLF | 11A4VLR |
| 11A5 | 11A5VHF | 11A5VHR | 11A5VLF | 11A5VLR |
| 11A7 | 11A7VHF | 11A7VHR | 11A7VLF | 11A7VLR |
| 11A11 | 11A11VHF | 11A11VHR | 11A11VLF | 11A11VLR |
| 11A12 | 11A12VHF | 11A12VHR | 11A12VLF | 11A12VLR |
| 12A1 | 12A1VHF | 12A1VHR | 12A1VLF | 12A1VLR |
| 12A2 | 12A2VHF | 12A2VHR | 12A2VLF | 12A2VLR |

A single bacterial colony containing the appropriate nucleic acid encoding the scFv in pCANTAB6 (WO 94/13804, FIGS. 19 and 20) was picked into each PCR reaction and the sample was amplified using the following parameters: 94° C. for 5 minutes, 94° C. for 1 min., 30 cycles of 55° C. for 1 min. and 72° C. 1 min., and 72° C. 5 min.

Digestion

The PCR products were cleaned up using a QIAquick™ 8-well purification kit (Catalog # 28144, Qiagen, Valencia Calif.) according to the manufacturer's directions. A 25 ul aliquot of the amplified VH PCR products was digested with BssHII and BstEII. A 25 ul aliquot of the amplified VL PCR products was digested with ApaLI and PacI.

The digested VH and VL PCR products were cleaned up using a QIAquick purification kit.

Ligation and Transformation

An aliquot of the cleaned up, digested PCR product was ligated into the appropriate vector digested with the same restriction enzymes. VH domains were ligated into pMON27816 (pEU1), and VL domains were ligated into either pMON27820 (pEU3) or pMON27819 (pEU4), depending on light chain class (Persic et al., Gene 187: 9-18, 1997). A portion of each of the ligation reactions was transformed into previously prepared chemically competent DH5α E. coli by heat shock and grown overnight on 2×TY agar plates containing Ampicillin.

Screening

Individual ampicillin resistant colonies were picked into liquid 2TY media (containing Ampicillin) in a 96-well plate and grown overnight. Once cultured, the colonies were screened by PCR to determine whether the vectors contained the appropriate domains. VH-containing plasmids were screened using the primers, PECSEQ1 and p95, and VL-containing plasmids were screened using the primers, PECSEQ1 and p156.

Colonies containing inserts were analyzed by DNA sequencing using the same primers as were used for the screening PCR.

Table 6 shows the oligonucleotide primers used to amplify the VH and VL domains.

TABLE 6

| | | |
|---|---|---|
| Oligo | | Function of |
| Name | Oligo Sequence (5′-3′) | Oligo |

| | | |
|---|---|---|
| AF32 | CTCTCCACAGGCGTGCACTCCTCGTCTG | Forward VL PCR |
| | AGCTGACTCAGGA SEQ ID NO:46 | primer for 7Ax |
| AF23 | CTATTCCTTAATTAAGTTAGATCTATTC | Reverse VL PCR |
| | TGACTCACCTAGGACGGTCAGCTTGGTC | primer for 7Ax |
| | CCTC SEQ ID NO:47 | |
| 7A2-VH-F | CTCTCCACAGGCGCGCACTCCGGGGTGC | Forward VH PCR |
| | AGCTGGTGCAGTC SEQ ID NO:48 | primer |
| 7A2-VH-R | TGAGGAGACGGTGACCATTGTCCCCTG | Reverse VH PCR |
| | SEQ ID NO:49 | primer |
| 7A4 VL-F | CTTTCTCTCCACAGGCGTGCACTCCTCT | Forward VL PCR |
| | GAGCTGACTCAGGACCCTGCT | primer |
| | SEQ ID NO:50 | |
| 7A4 VL R | CTATTCCTTAATTAAGTTAGATCTATTC | Reverse VL PCR |
| | TGACTCACCTAGGACGGTCAGCTTGGTC | primer |
| | CCTCCGCC SEQ ID NO:51 | |
| 7A5-VH-F | CTCTCCACAGGCGCGCACTCCGGGGTGC | Forward VH PCR |
| | AGCTGGTGGAGTC SEQ ID NO:52 | primer |
| 7A5-VH-R | TGAGGAGACG GTGACCAGGG | Reverse VH PCR |
| | TTCCCCG SEQ ID NO:53 | primer |
| 7A6-VH-F | CTCTCCACAGGCGCGCACTCCGAAGTGCA | Forward VH PCR |
| | GCAGTC SEQ ID NO:54 | primer |
| 7A6-VH-R | TGAGGAGACG GTGACCAGGG | Reverse VH PCR |
| | TGCCCTG SEQ ID NO:55 | primer |
| 8A1-VH F | GATCGATCGCGCGCACTCCGAGGTGCAG | Forward VH PCR |
| | CTGGTGCAGTCTG SEQ ID NO:56 | primer |
| 8A1-VH R | GATCGATCGGTGACCATGGTTCCTTGGC | Reverse VH PCR |
| | CCC SEQ ID NO:57 | primer |
| 8A1-VL F | GATCGATCGTGCACTCCTCTGAGCTGAC | Forward VL PCR |
| | TCAGGACCCTG SEQ ID NO:58 | primer |
| 8A1-VL R | GATCGATCTTAATTAAGTTAGATCTATT | Reverse VL PCR |
| | CTGACTCACCTAGGACGGTCAGCTTGGT | primer |
| | CCCTCCGCC SEQ ID NO:59 | |
| 9A1-VH F | GGATCTTGGCGCGCACTCCGAGGTGCAG | Forward VH PCR |
| | CTGGTGGAGTCTGG SEQ ID NO:60 | primer |
| 9A1-VH-R | GATCGATCGGTGACCATTGTCCCTCGGC | Reverse VH PCR |
| | CCCAGATATC SEQ ID NO:61 | primer |
| 9A1-VL-F | GATCGATCGTGCACTCCCAGTCTGTGCT | Forward VL PCR |
| | GACTCAGCCACC SEQ ID NO:62 | primer |
| 9A1-VL-R | GATCGATCTTAATTAAGTTAGATCTATT | Reverse VL PCR |
| | CTGACTCACCTAGGACGGTCAGCTTGGT | primer |
| | CCCTCC SEQ ID NO:63 | |
| 9A2-VH F | GATCGATCGCGCGCACTCCCAGGTCCAG | Forward VH PCR |
| | CTGGTGCAGTCT SEQ ID NO:64 | primer |
| 9A2-VH R | GATCGATCGGTGACCCAGGGTTCCTCGG | Reverse VH PCR |
| | CCCCAGTAG SEQ ID NO:65 | primer |
| 9A2-VL F | GATCGATCGTGCACTCCGCACTTAATTT | Forward VL PCR |
| | TATGCTGACT SEQ ID NO:66 | primer |
| 9A2-VL R | GATCGATCTTAATTAAGTTAGATCTATT | Reverse VL PCR |
| | CTGACTCACCTAGGACGGTGACCTTGGT | primer |
| | CC SEQ ID NO:67 | |
| 11A1-VH F | GATCGATCGCGCGCACTCCGAGGTGCAG | Forward VH PCR |
| | CTGGTGGAGTCT SEQ ID NO:68 | primer |
| 11A1-VH R | GATCGATCGGTGACCAGGGTGCCTTTGC | Reverse VH PCR |
| | CCCAGACAGG SEQ ID NO:69 | primer |
| 11A1-VL F | GATCGATCGTGCACTCCGCACTTTCCTA | Forward VL PCR |
| | TGTGCTGACTC SEQ ID NO:70 | primer |
| 11A1-VL R | GATCGATCTTAATTAAAAGTTAGATCTA | Reverse VL PCR |
| | TTCTGACTCACCTAGGACGGTGACCTTG | primer |
| | GTCCCTC SEQ ID NO:71 | |
| 11A2-VH F | GATCGATCGCGCGCACTCCGAGGTGCAG | Forward VH PCR |
| | CTGTTGGAGTCTG SEQ ID NO:72 | primer |
| 11A2-VH R | GATCGATCGGTGACCATTGTCCCCTGGC | Reverse VH PCR |
| | CCCAGACATC SEQ ID NO:73 | primer |
| 11A2-VL F | GATCGATCGTGCACTCCGCACTTTCTTC | Forward VL PCR |
| | TGAGCTGACTC SEQ ID NO:74 | primer |
| 11A2-VL R | GATCGATCTTAATTAAGTTAGATCTATT | Reverse VL PCR |
| | CTGACTCACCTAGGACGGTGACCTTGGT | primer |
| | CCCAC SEQ ID NO:75 | |
| 11A3-VH F | GATCGATCGCGCGCACTCCGAGGTGCAG | Forward VH PCR |
| | CTGGTGCAGTCGGGGGC | primer |
| | SEQ ID NO:76 | |
| 11A3-VH R | GATCGATCGGTGACCAGGGTGCCTCGGC | Reverse VH PCR |
| | CCCAGG SEQ ID NO:77 | primer |
| 11A3-VL F | GATCGATCGTGCACTCCGCACTTTCTTC | Forward VL PCR |
| | TGAGCTGACTCAGG SEQ ID NO:78 | primer |
| 11A3-VL R | GATCGATCTTAATTAAGTTAGATCTATT | Reverse VL PCR |
| | CTGACTCACCTAGGACGGTCAGCTTGGT | primer |
| | CCCTCCGCCGAACACC | |
| | SEQ ID NO:79 | |
| 11A4-VH F | GATCGATCGCGCGCACTCCGAGGTGCAG | Forward VH PCR |
| | CTGTTGGAGTCTG SEQ ID NO:80 | primer |
| 11A4-VH R | GATCGATCGGTGACCATTGTCCCTTGGC | Reverse VH PCR |
| | CCCAGGGG SEQ ID NO:81 | primer |
| 11A4-VL F | GATCGATCGTGCACTCCGCACTTTCCTA | Forward VL PCR |
| | TGAGCTGACTC SEQ ID NO:82 | primer |
| 11A4-VL R | GATCGATCTTAATTAAGTTAGATCTATT | Reverse VL PCR |
| | CTGACTCACCTAGGACGGTCAGCTTGGT | primer |
| | CCCGCCGCC SEQ ID NO:83 | |
| 11A5-VH F | GATCGATCGCGCGCACTCCCAGGTCCAG | Forward VH PCR |
| | CTGGTGCAGTC SEQ ID NO:84 | primer |
| 11A5-VH-R | GATCGATCGGTGACCAGGGTTCCTTTGC | Reverse VH PCR |
| | CCCAGGAGTC SEQ ID NO:85 | primer |
| 11A5-VL-F | GATCGATCGTGCACTCCGCACTTTCTTC | Forward VL PCR |
| | TGAGCTGACTC SEQ ID NO:86 | primer |
| 11A5-VL-R | GATCGATCTTAATTAAGTTAGATCTATT | Reverse VL PCR |
| | GTGACTCACCTAGGACGGTGACCTTGGT | primer |
| | CCCTCCGCCGAACACC | |
| | SEQ ID NO:87 | |
| 11A7-VH F | GATCGATCGCGCGCACTCCGAGGTCCAG | Forward VH PCR |
| | CTGGTGCAGTCTG SEQ ID NO:88 | primer |
| 11A7-VH R | GATCGATCGGTGACCATTGTCCCTCTGC | Reverse VH PCR |
| | CCCAGGAGTC SEQ ID NO:89 | primer |
| 11A7-VL F | GATCGATCGTGCACTCCGCACTTTCTTC | Forward VL PCR |
| | TGSGCTGACTCAG SEQ ID NO:90 | primer |
| 11A7-VL R | GATCGATCTTAATTAAGTTAGATCTATT | Reverse VL PCR |
| | CTGACTCACCTAGGACGGTGACCTTGGT | primer |
| | CCCTCCGCCG SEQ ID NO:91 | |
| 11A11-VH F | GATCGATCGCGCGCACTCCAGGTGCAGC | Forward VH PCR |
| | TGGTGGAGTCTGG SEQ ID NO:92 | primer |
| 11A11-VH R | GATCGATCGGTGACCAGGGTGCCCTGGC | Reverse VH PCR |
| | CCCAGGAGTC SEQ ID NO:93 | primer |
| 11A11-VL F | GATCGATCGTGCACTCCGCACTTAATTT | Forward VL PCR |
| | TATGCTGACTC SEQ ID NO:94 | primer |
| 11A11-VL R | GATCGATCTTAATTAAGTTAGATCTATT | Reverse VL PCR |
| | CTGACTCACCTAGGACGGTGACCTTGGT | primer |
| | CCCAGTTCCGAA SEQ ID NO:95 | |
| 11A12-VH F | GATCGATCGCGCGCACTCCGAGGTGCAG | Forward VH PCR |
| | CTGTTGGAGTCTG SEQ ID NO:96 | primer |
| 11A12-VH-R | GATCGATCGGTGACCATTGTCCCCCGGC | Reverse VH PCR |
| | CCCAATAATCAAAG SEQ ID NO:97 | primer |
| 11A12-VL F | GATCGATCGTGCACTCCGCACAGGCTGT | Forward VL PCR |
| | GCTGACTCAGC SEQ ID NO:98 | primer |
| 11A12-VL R | GATCGATCTTAATTAAGTTAGATCTATT | Reverse VL PCR |
| | CTGACTCACCTAGGACGGTGACCTTGGT | primer |
| | CCCGCCGCCGAACACCG | |
| | SEQ ID NO:99 | |
| 12A1-VH-F | GATCGATCGCGCGCACTCCGAGGTCCAG | Forward VH PCR |
| | CTGGTACAGTCTGG SEQ ID NO:100 | primer |
| 12A1-VH-R | GATCGATCGGTGACCAGGGTTCCTTTGC | Reverse VH PCR |
| | CCCAGG SEQ ID NO:101 | primer |
| 12A1-VL-F | GATCGATCGTGCACTCCGCACTTTCTTC | Forward VL PCR |
| | TGAGCTGACTCAGGACC | primer |
| | SEQ ID NO:102 | |
| 12A1-VL-R | GATCGATCTTAATTAAGTTAGATCTATT | Reverse VL PCR |
| | CTGACTCACCTAGGACGGTCAGCTTGGT | primer |
| | CCCTCC SEQ ID NO:103 | |
| 12A2-VH-F | GATCGATCGCGCGCACTCCGAGGTCCAG | Forward VH PCR |
| | CTGGTGCAGTCTGG SEQ ID NO:104 | primer |
| 12A2-VH-R | GATCGATCGGTGACCAGGGTGCCCTGGC | Reverse VH PCR |
| | CCCAGG SEQ ID NO:105 | primer |
| 12A2-VL-F | GATCGATCGTGCACTCCGCACTTTCTTC | Forward VL PCR |
| | TGSGCTGSCTCAG SEQ ID NO:106 | primer |
| 12A2-VL-R | GATCGATCTTAATTAAGTTAGATCTATT | Reverse VL PCR |
| | CTGACTCACCTAGGACGGTCAGCTTGGT | primer |
| | CCCTCC SEQ ID NO:107 | |

After the scFvs were converted to IgGs or Fabs the resulting antibodies were for example referred to as PINT-7A2 IgG and PINT-7A2 Fab.

Expression of IGF-1R MAb

Expression of the functional heavy chain gene cassette was driven by the GV promoter and terminated by the SV40 poly adenylation signal. The GV promoter is a synthetic promoter comprised of five repeats of the yeast Gal4 upstream activation sequence plus a minimal CMV promoter (Carey, M. et al., Nature 345 (1990), 361-364). The vector also contained the dhfr expression cassette from pSV2dhfr. Chinese hamster ovary (CHO/GV) cells transformed to express a chimeric transactivator (GV) derived from the fusion of the yeast Gal4 DNA binding domain and the VP16 transactivation domain (Carey, M. et al., Nature 345 (1990), 361-364) were transfected simultaneously with heavy-chain and light chain expression vectors using Lipofectamine 2000 (Gibco) according to the manufacturers instructions. Cell were grown at 37° C., 5% CO₂ in IMDM (Invitrogen)+10% FBS (Invitrogen)+1×HT supplement (Invitrogen) for forty-eight hours after transfection and then the cells were placed under selection by removing hypoxanthine and thymidine from the media (IMDM+10%/dialyzed FBS (Invitrogen)). After 10 days the pool of cells was cloned in 96-well plates and after 14 days in culture the 96-well plates were screened and the highest expressing clones were expanded. Expression was done in roller bottles by plating one confluent T75 flask into one 1700 cm² roller bottle containing 400 ml of IMDM+10% dialyzed FBS media.

Purification of IGF-1R MAb

Purification of IGF-1R immunoglobulins was accomplished by affinity chromatography utilizing 1 ml Amersham Fast Flow recombinant protein A columns. The columns were equilibrated with 20 mls of GIBCO PBS pH 7.4(#12388-013) at 1 ml per minute. Conditioned media containing anti IGF-1 R IgG was 0.2 micron filtered then applied to the equilibrated column at 0.5 ml per minute. Unbound protein was washed from the column with 60 ml of PBS at 1 ml per minute. The IgG was eluted with 20 ml of 0.1 M glycine plus 0.15 M NaCl pH 2.8 at 1 ml per minute. The eluate was collected into 2 ml of 1 M Tris Cl pH 8.3 with stirring. Amicon Centriprep YM-30 filtration units were used to concentrate the eluates (22 ml) to approximately 1.5 ml. The concentrates were dialyzed in Pierce 10K MWCO Slide-A-lyzer cassettes versus 2×1 L of PBS. Following dialysis the IgG was passed through a 0.2 micron filter, aliquoted and stored frozen at −80 C. IgG was characterized by reducing and non-reducing SDS PAGE, size exclusion chromatography and quantitated by absorbance at 280 nm using a calculated extinction coefficient of 1.45 OD units equals 1 mg/ml. A subset was additionally characterized by N-terminal amino acid sequencing and amino acid compositional analysis.

### Example 3

### Determination of Affinity Constants (Kd) of IGF-1R Monoclonal Antibodies by Surface Plasmon Resonance (BIAcore)

We measured the kinetics of binding of the antibodies to IGF1R using surface plasmon resonance, or BIAcore, technology. Antibodies were indirectly captured onto a BIAcore CM5 research grade sensor chip by two methods. Mobile phase buffer was Hepes-buffered saline (150 mM NaCl, 10 mM Hepes, 3.4 mM EDTA, 0.05% surfactant P-20, pH 7.4) for all experiments, and capture was performed at a flow rate of 5 μL/min. In the first capture method the sensor chip was activated with a 1:1 mixture of 400 nM N-ethyl-N-(dimethylaminopropyl)-carbodiimide (EDC) and 100 mM N-hydroxysuccinimide (NHS) for seven minutes. Following activation, protein A at 50 μg/mL in 10 mM acetate (pH 4.8) was injected for up to seven minutes, and unreacted groups were quenched with 1 M ethanolamine for seven minutes. For this method, fresh antibody is captured onto covalently-bound protein A prior to each determination. In an alternative capture method, mouse anti-human IgG was applied to the chip as described above for protein A.

Each experimental injection was conducted at a flow rate of 40 μL /min. IGF1R extracellular domain at 1-10 ug/ml was diluted into seven sample tubes at concentrations between 50 pM and 50 nM in mobile phase. Each injection was of one minute duration, followed by five minutes of mobile phase buffer for the measurement of the dissociation phase. Following injection and dissociation, the chip was regenerated with one to two minutes of 2.25 to 4.5 M magnesium chloride in water. Table 7 shows results corrected by subtracting the blank flow cell control from each injection, then simultaneously calculating the kinetics for all seven concentrations using BIAevluation software. A Langmuir fit with mass transfer curve fitting model was used in keeping with the nature of the antibody ligand interaction being tested.

TABLE 7

| IgG | Protein A K_{D}, pM | Anti-human IgG K_{D}, pM |
|---|---|---|
| 8A1 | 109 | 503 |
| 9A2 | 240 | 138 |
| 11A4 | ND | 913 |

ND = not determined

### Example 4

### Antibody-Mediated Blockade of IGF-I/IGF-II Binding to IGF-1R

Experiments to determine the ability of antibodies of the invention to inhibit IGF-I or IGF-II binding to IGF-IR were performed in 48-well tissue culture dishes (Corning, #3548). NIH-3T3 fibroblasts expressing the human IGF-IR, or NIH-3T3 non-transfected fibroblasts were plated at 6×10⁴ cells per well in 0.5 ml of DMEM (Gibco, #11960-044) supplemented with 10% heat-inactivated fetal bovine serum (Gibco, #16140-071), 2 mM L-glutamine (Gibco, #25030-081) and 50 U/ml penicillin-streptomycin (Gibco, #15070-063). The NIH-3T3 cells were used as a control for non-specific cell binding. The plates were incubated at 37° C./5% CO₂ for 24 hours to allow cells to attach and become 80-90% confluent. The overlying media was then replaced with 0.5 ml per well of starvation media consisting of DMEM, 20 mM Hepes (Gibco, #15630-080), 2 mM L-glutamine and 0.1% bovine serum albumin (Equitech-Bio, protease-free, Kerrville, Tex.) and the plates were incubated at 37° C., 5% CO₂ overnight. All subsequent binding steps were conducted at 4° C. Test antibodies were diluted in ice-cold starvation media to the desired final concentration and 100 μl added per well. All samples were performed in duplicate. After 30 minutes, IGF-I (Perkin-Elmer, #NEX241) or IGF-II (Amersham, #IM238) radioligand binding was initiated by addition of 200 μM radioligand in 100 μl per well, and binding was conducted for a further 2.5 hours. Cell monolayers were washed three times with ice-cold PBS (Gibco, #14040-117) and cells and associated radioactivity were released by adding 0.5ml 2% sodium dodecyl sulfate/0.2N NaOH to each well and heating the plates at 60° C. for 15 minutes. Lysate associated radioactivity was quantitated by gamma scintillation spectrometry. Alternatively, the same described experiment was performed with preincubation with the test antibodies at 37° C. for 10 minutes, followed by 10 minutes incubation at 37° C. after addition of 400 pM of the iodinated radioligand.

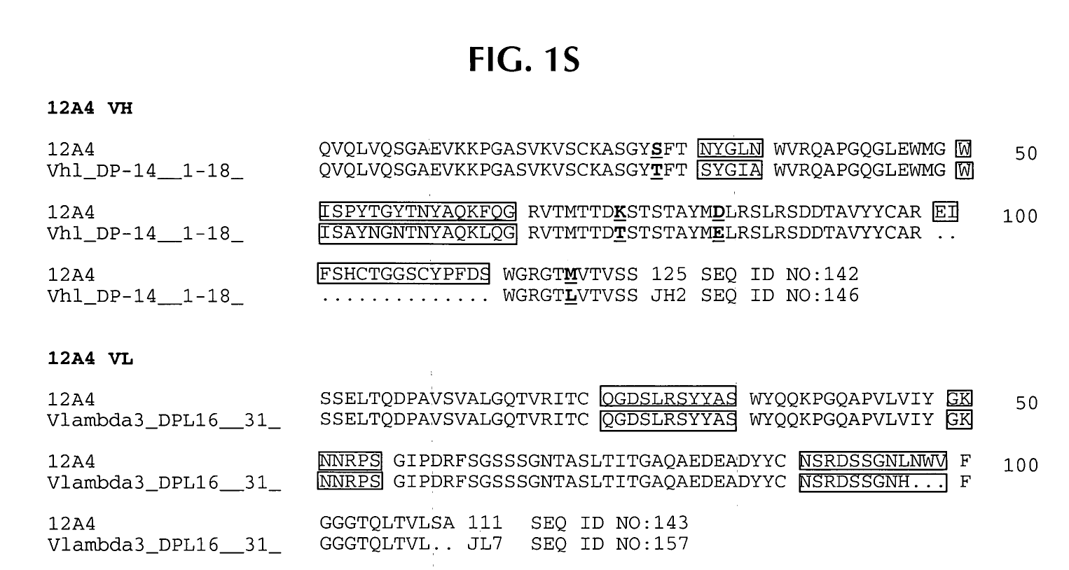

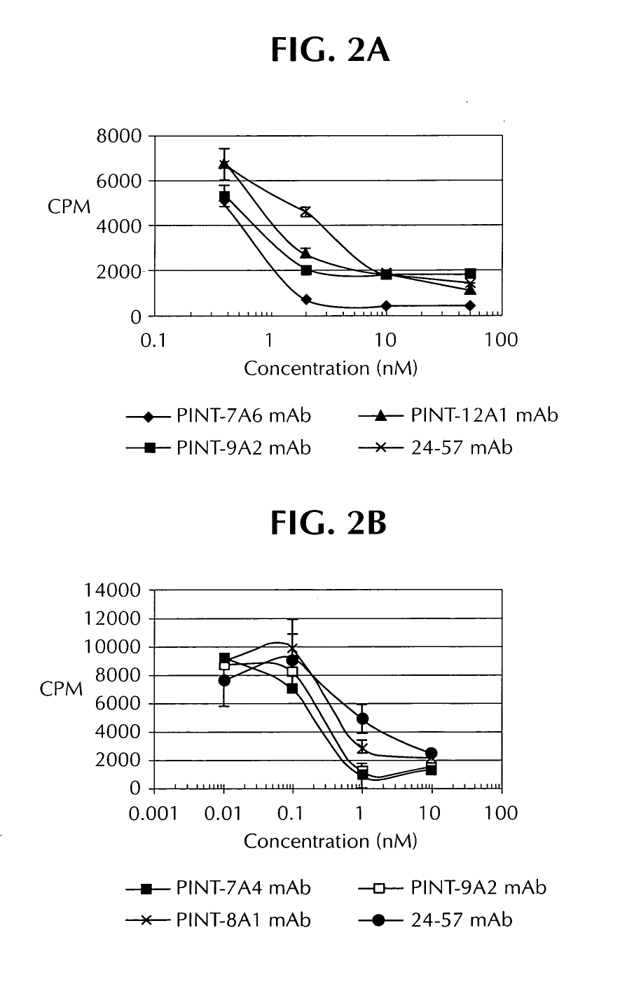

FIG. 2 shows representative graphs of the competition binding experiment with IGF-1R antibodies 7A6, 9A2, and 12A1 inhibiting [¹²⁵I]-labeled IGF-1 binding and IGF-1R antibodies 7A4, 8A1, and 9A2 inhibiting [¹²⁵I]-labeled IGF-2 binding at 4° C. on NIH 3T3-fibroblasts expressing the human IGF-1R.

Table 8 shows the IC50 values obtained for the IGF-1R antibodies. Commercially available IGF-1R antibodies 24-57 (#MS-643-PABX, NeoMarkers, Fremont Calif. and αIR3 (#GR11SP2, Oncogene Research Products, San Diego, Calif.) were used as controls. MOPC-21 (#M-7894, Sigma) was used as an IgG1 isotype control and UPC-10 (#F-0528) was used as an IgG2a isotype control.

TABLE 8

| | IC50 (nM) | IC50 (nM) | IC50 (nM) | IC50 (nM) |
|---|---|---|---|---|
| | IGF-1 | IGF-1 | IGF-II | IGF-1I |
| | Competition | Competition | Competition | Competition |
| IgGs | (4° C.) | (37° C.) | (4° C.) | (37° C.) |
| 7A2 | 0.5, 1.3, 0.5 | | <0.8, 0.4 | |
| 7A4 | <0.4, 0.26 | | <0.8, 0.2 | |
| 7A6 | 1, 0.8 | | <0.8, 0.3 | |
| 8A1 | <0.4, 0.13 | 0.9, 1.4 | <0.8, 0.3 | 1.5, 2.4 |
| 9A2 | 1, 0.7, 1.4, 1.1 | 2.3, 2.3 | <0.8, 0.5 | 5, 4 |
| 11A1 | 8, 10 | | >75, >75 | |
| 11A2 | >50, >100 | | | |
| 11A3 | 1.1, 1.2 | | | |
| 11A4 | >50, >100, >50 | 1.9, 1.9 | >75, >75 | 3, 2.6 |
| 11A5 | 1.2, <0.4, 0.75 | | | |
| 11A7 | 1, 1.4 | | | |
| 11A11 | 1.6, 1.6 | | | |
| 11A12 | 32, 8, 6 | | | |
| 12A1 | 1.5, 1.2 | | | |
| 12A2 | 1, 0.7 | | | |
| 12A3 | 1.5, 1.7 | | | |
| 12A4 | >50, >50 | | | |
| 24-57 | 3, 1.5, 4 | 1.7, 1.9 | 1, 0.6 | 4, 6.5 |
| 24-60 | 3 | 1.3, 1.3 | >100, >100 | >50, >50 |
| Alpha IR3 | 3.5 | 1.6, 1.9 | >100, >100 | >50, >50 |
| MOPC-21 | >50, >100, >50 | >50, >50 | >100, >100 | >50, >50 |
| UPC-10 | >50 | | >100, >100 | >50, >50 |
| IGF-I | 0.5, 0.25 | 1, 0.9 | 0.3 | |
| IGF-II | | | 1.8, 2 | 1.3, 2.4 |

### Example 5

### Antibody-Mediated Blocking of Insulin/Insulin-Receptor Binding

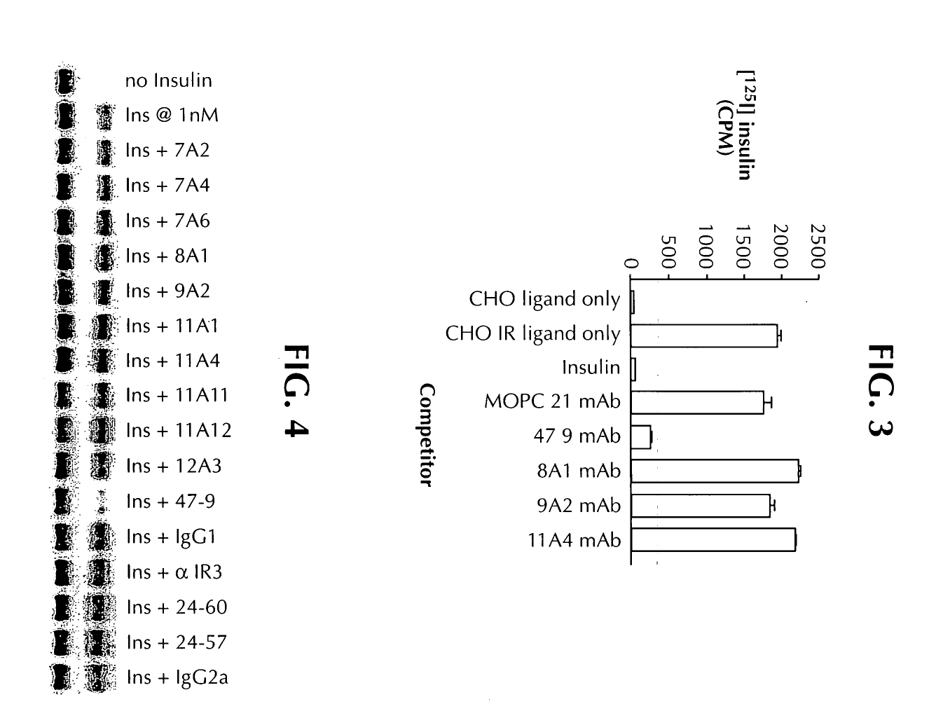

Experiments to test the ability of the monoclonal antibodies of the invention to inhibit insulin binding to the insulin-receptor were performed in a 48-well tissue flat bottom culture treated plate (Corning, #3548) cell-based assay. Human IGF-IR transfected Chinese hamster ovary (CHO) or parental (untransfected) CHO cells were plated at 6×10⁴ cells per well in 500 μl of IMDM (Gibco, #12440-053) supplemented with 10% heat-inactivated fetal bovine serum (Gibco, #16140-071), 2 mM L-glutamine (Gibco, #25030-081), 100 μM sodium hypoxanthine+1.6 μM thymidine; HT Supplement (Gibco, #11067-030). The Parental 3T3 cells were used as a control for background radioactivity. We then incubated the plates at 37° C., 5% CO₂ for 24 hours to allow cells to attach and become 80-90% confluent. The media was decanted from the plates, replaced with 500 μl per well of starvation or assay media consisting of IMDM, 20 mM Hepes (Gibco, #15630-080), 2 mM L-glutamine and 0.1% bovine serum albumin (Equitech-Bio, protease-free, Kerryville, Tex.) and the plates were incubated at 37° C., 5% CO₂ overnight. The antibodies were diluted in cold assay media to the desired final concentration and added 100 μl per well. All samples were performed in duplicate. The plates were incubated at 4° C. for 30 minutes. [125I]-Porcine Insulin Receptor (Perkin Elmer, #NEX104) was diluted to a concentration of 100 pM in cold assay media and 100 μl was added per well. The plates were incubated for 2.5 hours at 4° C., then aspirated the media and washed 3× with cold DPBS (Gibco, #14040-117). The cells were lysed by adding 500 μl 0.2 NaOH, 2% SDS and incubating the plates for 15 minutes at 60° C. The samples were transferred to 12×75 tubes (Sarsted, #55.476, 5 ml) and the signal read on a gamma counter. FIG. 3 shows that IGF-IR antibodies 8A1, 9A2, and 11A4 do not inhibit binding of insulin to the CHO cells expressing the human insulin receptor. All of the antibodies of the invention were tested and all had IC50s greater than 200 nM. Insulin Receptor mouse monoclonal antibody 47-9 (#MS-633-PABX, NeoMarkers, Fremont, Calif.) was used as a positive control in the experiment.

### Example 6

### Inhibition of Insulin Receptor Activation by IGF-1R Antibodies

Although none of the antibodies of the invention significantly block binding of insulin to Chinese hamster ovary (CHO) cells over-expressing the full-length human insulin receptor, we wanted to ensure that antibodies of the invention did not prevent insulin-induced insulin receptor tyrosine phosphorylation and activation. To this end, we plated CHO cells expressing the human insulin receptor in 6 well clusters in complete media (IMDM (Gibco, #12440-053) supplemented with 10% beat-inactivated fetal bovine serum (Gibco, #16140-071), 2 mM L-glutamine (Gibco, #25030-081), 100 μM sodium hypoxanthine+1.6 μM thymidine; HT Supplement (Gibco, #11067-030) and about 80% confluent wells were starved overnight at 37° C./5%CO₂ with the above media containing 0.5% BSA vs. fetal bovine serum. Dishes were placed in a 37° C. circulating water bath and 2m1 fresh starvation media added together with no insulin, or human insulin (Sigma, 1 nM final concentration) together with 100 nM of test antibodies. After 15 min at 37° C., the dishes were chilled on ice water and washed three times with ice-cold PBS. Cells were lysed and scrape-harvested in 0.3 ml lysis buffer (1% Nonidet P-40, 25 mM Tris-HCl, pH 7.5, 10% glycerol, 0.15 M NaCl, 5 mM EDTA, phosphatase inhibitors (Sigma P-2850, P-5726) and protease inhibitor (Sigma P-8340) cocktails). Lysates were clarified by centrifuging at 10,000×g for 20 min, and then equivalent aliquots of the supernatant fraction were separated by SDS-PAGE (4-12% Nu-PAGE gels, Bis-Tris, MOPS buffer, Invitrogen) under reducing conditions and transferred to nitrocellulose (BA-83, Schleicher and Schuell). Membranes were probed with antibody to insulin receptor beta chain (sc-711, Santa Cruz Biotechnology), phosphotyrosine insulin receptor kinase domain (#44-802, Biosource), or actin (Sigma A-2066) for total protein loading. As shown in FIG. 4, under equivalent protein loading conditions for actin and total insulin receptor phosphorylation of the kinase domain of insulin receptor was observed upon insulin addition to cells, and only the positive control insulin receptor blocking antibody (MS-633-PABX, Lab Vision) significantly inhibited tyrosine phosphorylation of the insulin receptor at 1000-fold molar excess to insulin. Hence, the antibodies of the invention inhibit neither insulin binding nor insulin-mediated receptor tyrosine kinase phosphorylation on intact human insulin receptor in vitro.

### Example 7

### Saturable and Specific Binding of IGF-1R mAbs-3T3 hu-IGF-1R Fibroblasts

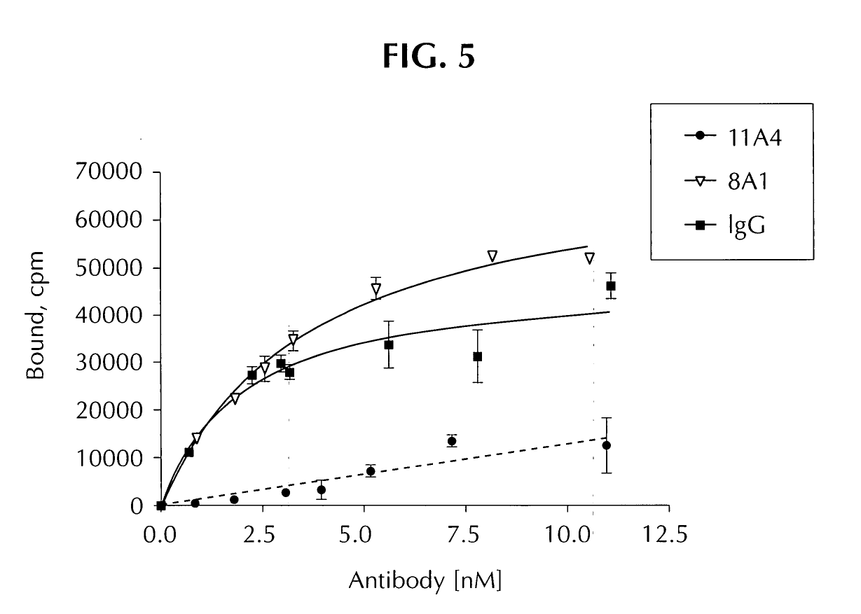

Experiments to test the ability of the monoclonal antibodies of the invention to bind directly to mouse NIH-3T3 cells transfected with the human IGF-1 receptor were performed in a saturable, and specific manner. Monoclonal antibodies 11A4 and 8A1, and human IgG, as a negative control, [¹²⁵I]-iodinated in house with Iodogen to specific activities of 19.2 μCi/μg protein, 17.5 μCi/μg protein, and 16.1 μCi/μg protein respectively. Exponentially growing human IGF-1 receptor-transfectant NIH-3T3 cells were used. To determine the total binding, various concentrations of [¹²⁵I]-iodinated monoclonal antibodies or control IgG were mixed with 10⁴ human IGF-1 receptor-transfectant NIH-3T3 cells, which had been dissociated from cell culture flasks (Costar Cat. No. 3151) with non-enzymatic cell dissociation solution (Gibco Cat. No. 13151-014), in 50 μl of ice-cold Hanks' Balanced Salt Solution (Gibco Cat. No. 14170-112) containing 0.2% BSA (Sigma Cat. No. A-7888) and 20 MM Hepes (Gibco Cat. No.15630-106) in non-stick microcentrifuge tubes (VWR Cat. No. 20170-650) in triplicates. The mixtures were in incubated on ice for 70 min. After the incubation the tubes were centrifuged at 1000 rpm for 1 min and the supernatant fractions were removed by aspiration. The cell pellets were washed with 50 μl of ice-cold Hanks' Balanced Salt Solution containing 0.2% BSA and 20 mM Hepes and centrifuged at 1000 rpm for min and the supernatant fractions were removed by aspiration. The resulting cell pellets were counted in Perkin Elmer Cobra Quantum gamma counter. The non-specific binding was determined in an identical fashion as the total binding determination, except, in addition to corresponding concentrations of [¹²⁵I]-iodinated monoclonal antibodies or control IgG, 200-fold excess of cold monoclonal antibodies or control IgG were mixed with 10⁴ cells of the human IGF-1 receptor-transfectant NIH-3T3 cell. The specific binding was obtained by subtracting the non-specific binding counts from the total binding counts in corresponding pairs. FIG. 5 is a representative graph that shows saturable and specific binding of 11A4 and 8A1 monoclonal antibodies to the human IGF-1 receptor-transfectant NIH-3T3 cell in contrast to the control IgG. The Kds for 11A4, 8A1 and IgG isotype control were 2.238, 4,008, and 186.2 respectively.

### Example 8

### Inhibition of IGF-1 Dependent Cell Proliferation

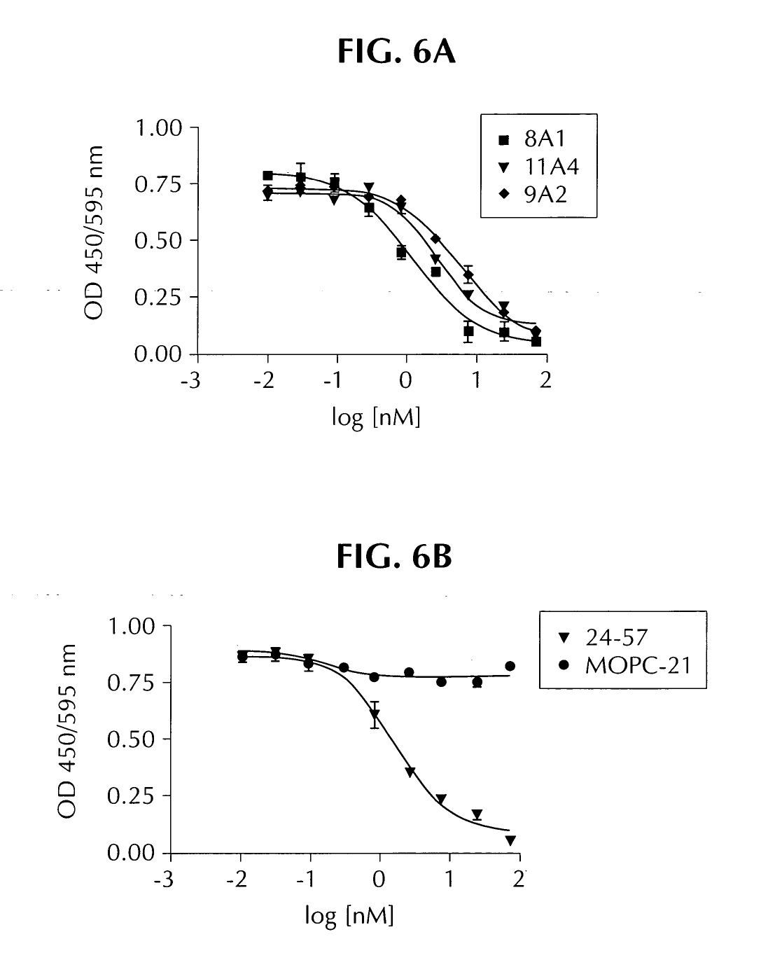

To evaluate whether or not addition of IgG versions of IGF-1R monoclonal antibodies could block DNA synthesis of 3T3-hu-IGFR-1R fibroblast, IGF-1R-transfected NIH-3T3 cells were plated at a cell density of 2×10⁴/well into a 96-well U-bottom plate in 100 μl of starvation media, DMEM high glucose media (Gibco, #11960-051), supplemented with 2 mM L-glutamine (Gibco, #25030-081), 20 mM 4-(2-hydroxyethyl)-1-piperazineethanesulfonic acid (Gibco, #15630-080; Hepes), and 0.1% protease-free bovine serum albumin (Equitech-Bio, protease-free, Kerrville, Tex.). Plates were incubated at 37° C./5% CO₂ overnight to allow the cells to attach. 50 ul of the starvation media was removed from the plates using a multi-channel and replaced with 50 ul fresh pre-warmed starvation media/well. The IGF-1R antibodies and recombinant human insulin growth factor-1 (rHu IGF-1, Equitech-Bio, #HIG-1100, lot #HIG90-139), were diluted to four times the desired final concentration in starvation media and added 25 μl of each per well. All samples were performed in duplicate. The plates were incubated at 37° C. for 48 hours. During the last 16 hrs of stimulus 10 μl of diluted BrdU labeling solution (Roche, cat #1647229, Cell Proliferation Elisa, BrdU, colorimetric) was added, to all wells (10 μM final conc.). The media was decanted by inverting the plates and blotting gently onto a paper towel. Plates were then dried at 60° C. for 1 hour. Fix Denat solution (Roche, cat #1647229) was then added at 200 ul per well and incubated 30-45 minutes at room temperature. Plates were then decanted again onto a paper towel and 200 μl of Dulbecco's PBS (Gibco, #14040-117) containing 2% BSA (Equitech-Bio) was added to each well to block for 30 minutes at room temperature. PBS was decanted and 100 ul of anti-BrdU-POD (monoclonal antibody, clone BMG 6H8, Fab fragment conjugated with peroxidase) was added per well and incubated for 90 minutes at room temperature. Decanting and tapping the plate onto a paper towel removed the antibody conjugate. The plates were rinsed 3× with 275 ul/well washing solution (Roche, cat #1647229). 100 μl/well of TMB substrate solution (tetramethyl-benzidine, Roche, cat#1647229) was added to the wells and incubated at room temperature for 5-30 minutes. 25 ul of 1M H₂SO₄ (VWR, #VW3232-1) was added and incubated approximately 1 minute with thorough mixing to stop further plate development. The optical density was measured on an ELISA plate reader (Bio-Rad, Model #3550) at 450 nm against a reference wavelength of 595 nm. FIG. 6 is a representative graph that displays IGF-1R antibodies 8A1, 9A2, and 11A4 ability to inhibit proliferation of IGF-1 driven NIH 3T3-fibroblasts that express the human IGF-1R.

Table 9 indicates the ability of the IGF-1R antibodies of the invention to inhibit IGF-1 dependent proliferation of these cells under assay conditions.

TABLE 9

| IgG | IC50 (nM) |
|---|---|
| 7A2 | 5, 3.8 |
| 7A4 | 0.73, 0.27 |
| 7A6 | >70, >70 |
| 8A1 | 0.41, 0.23 |
| 9A2 | 6.7, 7.0 |
| 11A1 | 5.4, 3.3 |
| 11A2 | >70, >70 |
| 11A3 | 32.1, >70 |
| 11A4 | 3.4, 2.6 |
| 11A5 | >70, >70 |
| 11A7 | >70, >70 |
| 11A11 | 16.4, 25 |
| 11A12 | >70, >70 |
| 12A1 | >70, >70 |
| 12A2 | >70, >70 |
| 12A3 | >70, >70 |
| 12A4 | >70, >70 |
| 24-57 | 2, 1.7, 0.9, 0.5 |
| 24-60 | 10 |
| Alpha IR3 | >70 |
| MOPC-21 | >70 |
| UPC-10 | >70 |

### Example 9

### Antibody-Mediated Inhibition of IGF-I-induced Tyrosine Phosphorylation or Antibody-mediated Enhancement of Tyrosine Phosphorylation of IGF-1R

ELISA experiments were performed in order to determine whether the antibodies of the invention were able to block IGF-I-mediated tyrosine phosphorylation/activation of the IGF-1R, or if IGF-1R antibodies of the invention could enhance phosphorylation/activation of the IGF-1R in the absence of IGF-1. IGF-I-mediated activation of the IGF-I R was detected by increased receptor-associated tyrosine phosphorylation.

ELISA Plate Preparation

ELISA 96-well capture plates were prepared by coating wells with 200 ng of mouse anti-IGF-1R monoclonal antibody (NeoMarkers, #MS-641-PABX) in 100 ul phosphate buffered saline [PBS] overnight at 4° C. Unoccupied binding sites were blocked by adding 200 ul blocking buffer (1% bovine serum albumin [BSA] in Tris-buffered saline [TBS]) for 2 hours at room temperature. The plates were washed three times with wash buffer (TBS+0.05% Tween 20), blotting the plates on paper towels between washes.

Preparation of Lysate from IGF-1R-Expressing Cells

NIH-3T3 cells expressing the human IGF-1R were plated at 3×10⁴/well in 100 ul serum-free media (DMEM high glucose media supplemented with 2 mM L-glutamine, 20 mM Hepes, and 0.1% BSA) in 96-well plates. The plates were incubated at 37° C., 5% CO₂ overnight to allow cell attachment. The media was decanted and replaced with 100 ul serum-free media containing the desired concentration of anti-IGF-1R antibodies. All determinations were performed in triplicate. The plates were incubated at 37° C. for one hour. The cells were stimulated by addition of 20 ul per well of 60 nM human IGF-1 (Equitech-Bio; Kerrville, Tex.) or alternatively, incubated without adding the human IGF-1 to test for agonism of the antibodies in the absence of IGF-1. The plates were incubated at 37° C. for 10 minutes. The media was decanted by inverting the plates and blotting gently onto paper towels the cells washed three times with PBS at 4° C. The cells were lysed by adding 150 ul per well of lysis buffer (M-PER mammalian protein extraction reagent [Pierce], containing 5 mM EDTA, protease (Sigma, P-8340), and phosphatase (Sigma, P-2850 and P-5726) inhibitor cocktails. Lysates were mixed by multiple pipetting prior to transferring 100ul of lysate from each well to the ELISA capture plates as described above. The plates were incubated for 2 hours at room temperature.

ELISA with Anti-Phosphotyrosine Antibodies

The cell lysate was removed by inverting the plates, the plates were washed three times with wash buffer and blotted on paper towels. 100 ul per well of a 1/1000 dilution of anti-phosphotyrosine antibody conjugated to horseradish peroxidase (4G10-HRP) was added and the plates for one hour at room temperature. The plates were washed six times with wash buffer and blotted on paper towels. We detected plate binding of 4G10-HRP by adding 100 ul per well of TMB (Sigma, T-4444) and plate development was allowed to proceed for 2-5 minutes at room temperature in the dark. We stopped the color development reaction by adding 100 ul 1N HCl to each well. Optical density was determined at 450 nm vs. 595 nm as a reference wavelength using an ELISA plate reader (Bio-Rad, Hercules Calif.).

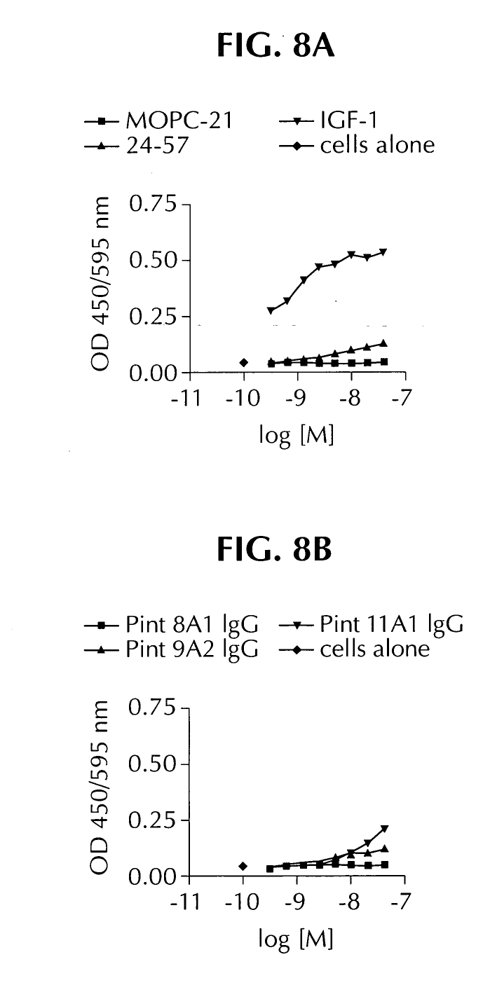

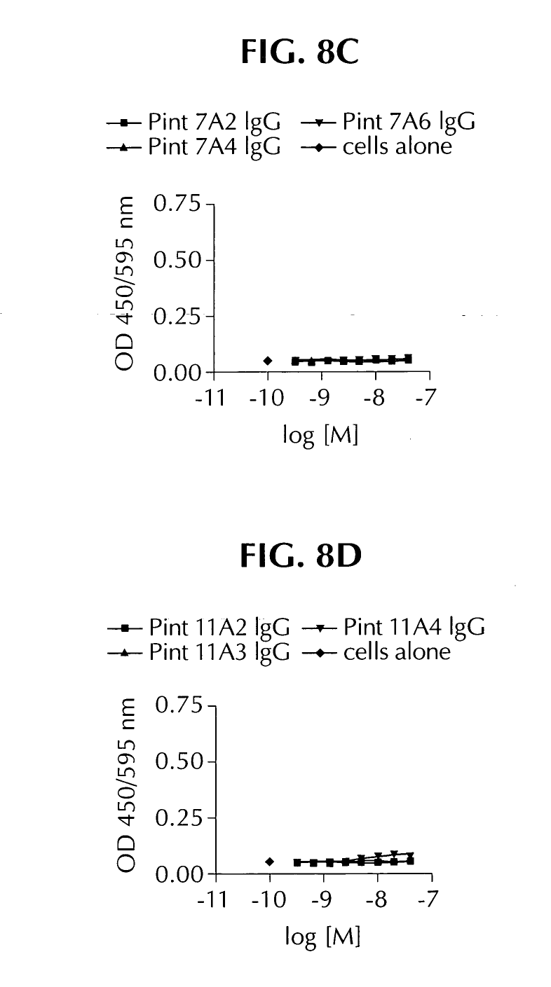

The results for the agonist version of the assay are shown in FIG. 8. The IGF-1R antibodies of the invention show minimal or no ability to phosphorylate the receptor on NIH 3T3-fibroblasts expressing the human IGF-1R.

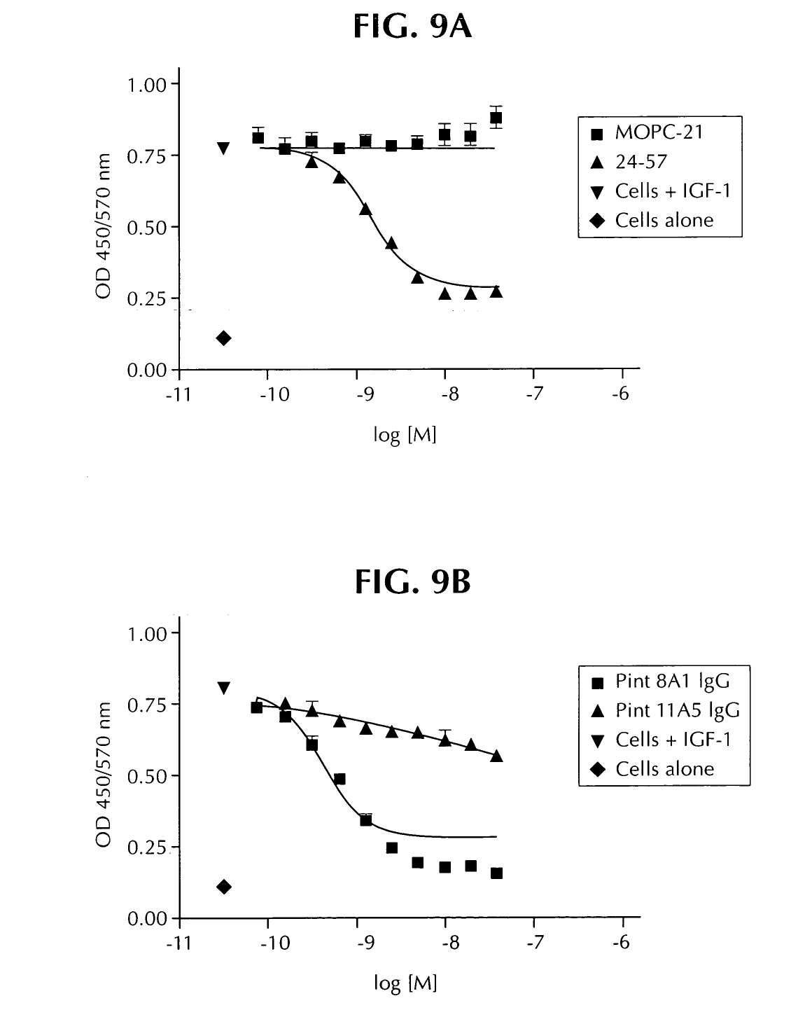

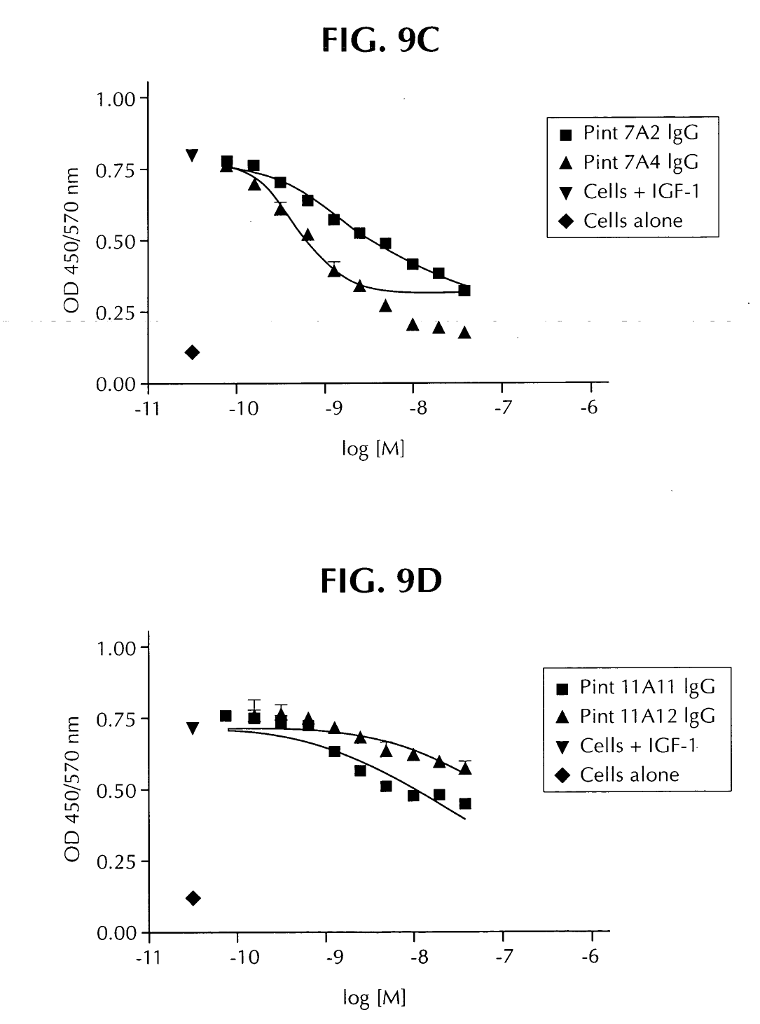

The results of at least two independent ELISA experiments with several antibodies of the invention are shown in Table 10. These experiments demonstrated the ability of the invention anti-IGF-1R antibodies to block IGF-1-mediated IGF-1R tyrosine phosphorylation. FIG. 9 shows representative graphs with IGF-1R antibodies of the invention 7A2, 7A4, 8A1, 11A5, 11A11, and 11A12 and the inhibition seen with this assay.

TABLE 10

| | Antagonist Assay |
|---|---|
| IgGs | IC50 (nM) |
| 7A2 | 2.62, 1.97 |
| 7A4 | 0.47, 0.46 |
| 7A6 | 9.7, 8.5 |
| 8A1 | 0.51, 0.43 |
| 9A2 | 1.6, 2.2, 1.89 |
| 11A1 | 5.4, 7.4 |
| 11A2 | >40 |
| 11A3 | 11.4, >40 |
| 11A4 | 2.4, 3.3 |
| 11A5 | >40 |
| 11A7 | 26.6, >40, 25.5 |
| 11A11 | 14.9, 10.6 |
| 11A12 | >40 |
| 12A1 | >40 |
| 12A2 | >40 |
| 12A3 | >40 |
| 12A4 | >40 |

### Example 10

### Effect of IGF-1 R Monoclonal Antibodies on IGF-1R Tyrosine Phosphorylation

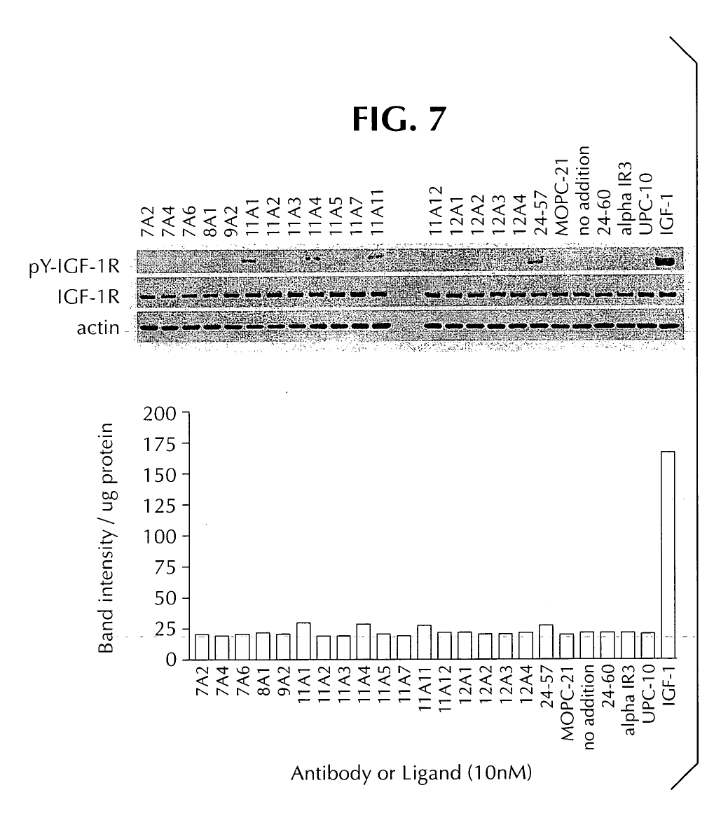

Having shown the ability of antibodies of the invention to block ligand-dependent tyrosine phosphorylation of the IGF-1R, we evaluated the ability of antibodies of the invention to directly stimulate tyrosine phosphorylation of the IGF-1R upon binding to the IGF-1R on cells. For this purpose, 12-well clusters of NIH-3T3 fibroblasts expressing the human IGF-1R were grown to about 80% confluence in 12-well tissue culture dishes in DMEM containing 20 mM Hepes and 10% FBS. Media was replaced overnight with the above media containing 0.1% BSA instead of serum. Dishes were placed in a 37° C. water bath and stimulated with 10 nM of IGF-1 or test monoclonal antibodies for 10 minutes. Dishes were then placed on ice-water, washed three times with ice-cold PBS, and cell lysates prepared by scrape-harvesting the cells from each well in 75 ul 1% Nonidet P40, 25 mM Tris-HCl (pH 7.5), 0.15M NaCl, 5 mM EDTA, 10% glycerol, and protease and phosphatase inhibitor cocktails. Lysates were clarified by centrifuging the scraped suspension at 10,000×g for 20 minutes at 5° C., and then 2 ul of each supernatant fraction was assayed for total protein by the Bradford method, using BSA as a standard. Known volumes of the clarified cell lysates were then subjected to SDS-PAGE on 4-12% Nu-PAGE gels (Novex) and transferred to nitrocellulose. Phosphorylated IGF-1R was detected by incubation of Westernblots with rabbit anti-pY-IGF-1R (Biosource #44-804) and detection with goat anti-rabbit IgG-HRP (Jackson Immunoresearch) and Supersignal as per manufacturers instructions. Exposures of 20 seconds on BioMax MR-1 film were scanned for band intensity using a Molecular Dynamics laser densitometer and analyzed with ImageQuant software. The band intensity (volume) was divided by the total protein loaded for each sample to determine the extent of IGF-1R tyrosine phosphorylation versus no treatment or isotype control antibodies. FIG. 7 shows minimal or no ability of the IGF-1R antibodies to phosphorylate the receptor on NIH 3T3-fibroblasts expressing the human IGF-1R. The results of this experiment indicated that most antibodies of the invention showed no detectable ability to induce phosphorylation of the IGF-1R when compared to control antibodies. Those IGF-1R antibodies that did show measurable agonist activity against the IGF-1R (e.g., 11A1, 24-57) were much less effective than IGF-I at stimulating IGF-1R tyrosine phosphorylation.

### Example 11

### Endocytosis of IGF-1R by IGF-I or Monoclonal Antibodies

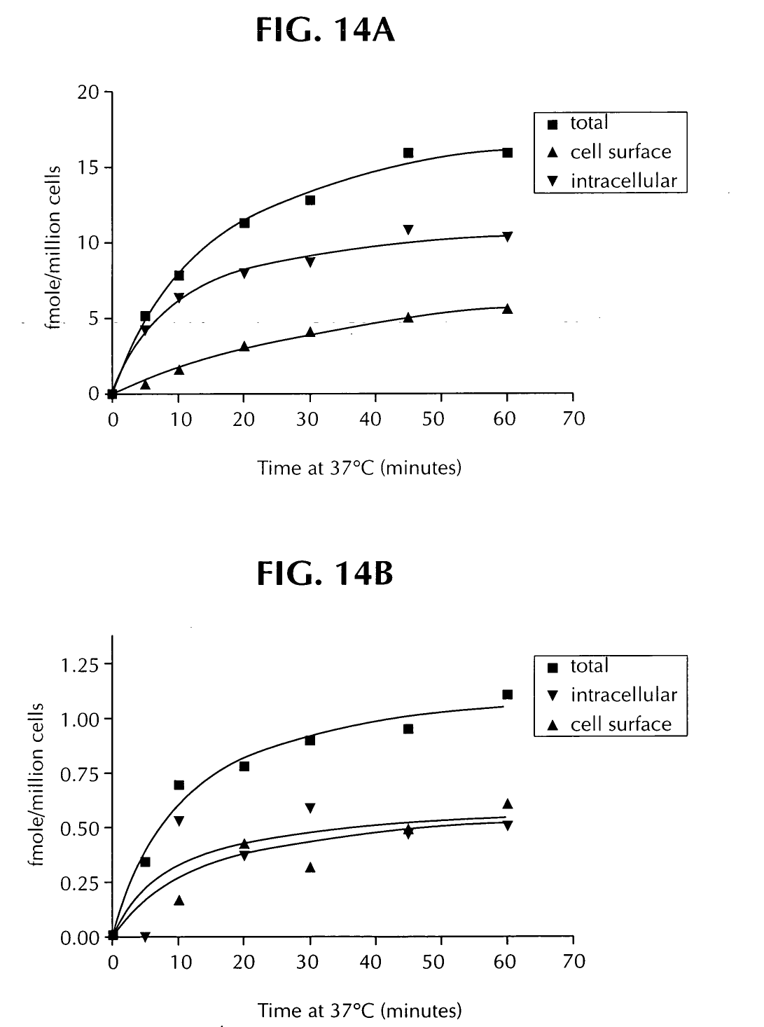

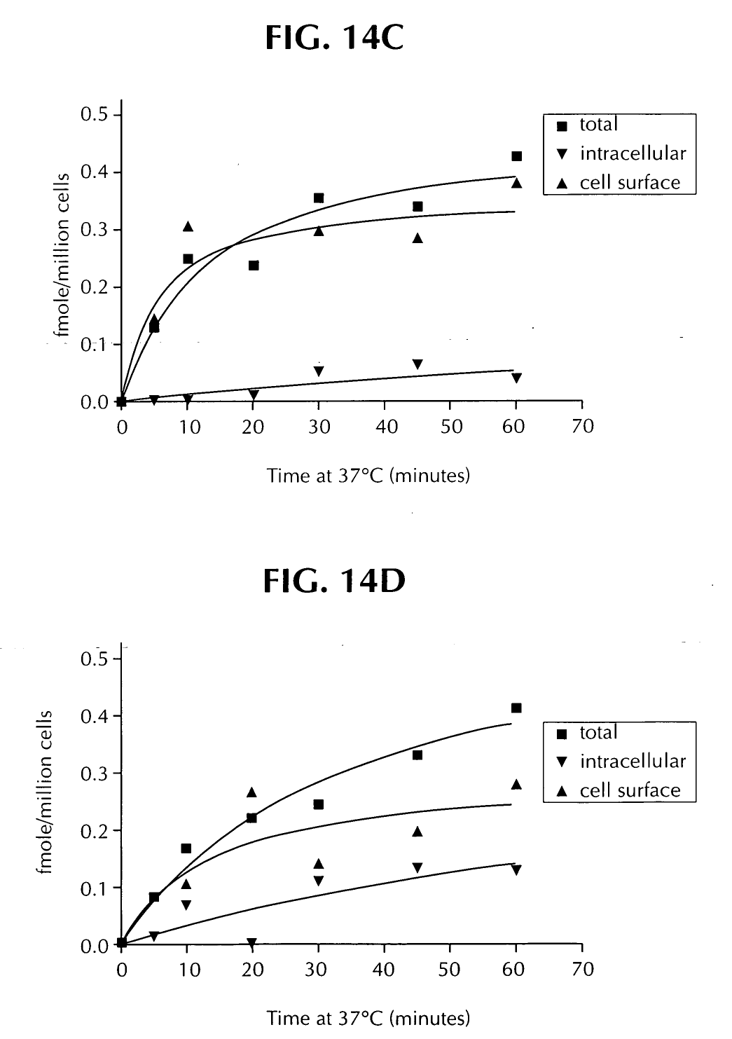

We examined the rate of intracellular accumulation of IGF-1R by indirectly measuring the intracellular accumulation of [¹²⁵I]-labeled monoclonal antibodies of the invention compared to [¹²⁵I]-labeled IGF-I. We focused these experiments on a subset of the antibodies of the invention, particularly 8A1, 9A2, and 11A4. For this purpose, 24-well clusters containing 5.0×E5 DU145 human prostate cancer cells expressing the human IGF-1R were cultured overnight in 0.5 ml per well of RPMI-1640 containing 20 mM Hepes and 0.2% BSA. Monolayers were incubated in a 37° C. water bath for up to one hour with 0.3nM of test monoclonal antibodies or IGF-I. Dishes were placed on ice water to inhibit further internalization of antibody or ligand and cell monolayers were washed four times over a 20 min period with ice-cold PBS adjusted to pH 2.0 with concentrated HCl, or with ice-cold PBS at pH 7.4 as a control. The low-pH wash step effectively removes greater than 95% of cell-surface bound radiolabeled antibodies or IGF-I from the cells at 4° C. Subsequently, well-associated radioactivity and cells were collected in 0.75 ml per well of 2% sodium dodecyl sulfate supplemented with 0.2N NaOH, and cell lysate radioactivity was quantitated by gamma scintillation spectrometry. Total monoclonal antibody or ligand binding was defined as cell-associated radioactivity retained following washing of cells with PBS at pH 7.4. Intracellular monoclonal antibody or ligand was defined as cell-associated radioactivity retained following washing of cells with PBS at pH 2.0. Cell-surface associated monoclonal antibody or ligand binding was defined as the difference between total and intracellular binding. FIG. 14 shows the rate of intracellular accumulation of IGF-1R by indirectly measuring the intracellular accumulation of[¹²⁵I]-labeled monoclonal antibodies 8A1, 9A2, and 11A4 compared to [¹²⁵I]-labeled IGF-1. The binding isotherms shown in FIG. 14 indicate that endocytosis and intracellular accumulation of IGF-I and the test monoclonal antibodies follow receptor binding at 37° C., albeit at different rates.

### Example 12

### IGF-1R Down Regulation

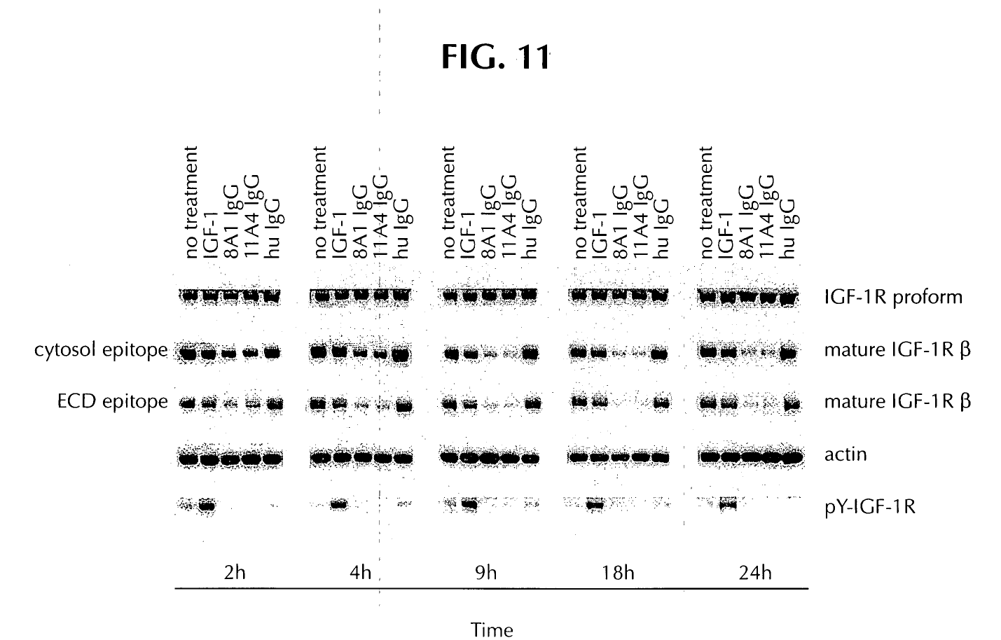

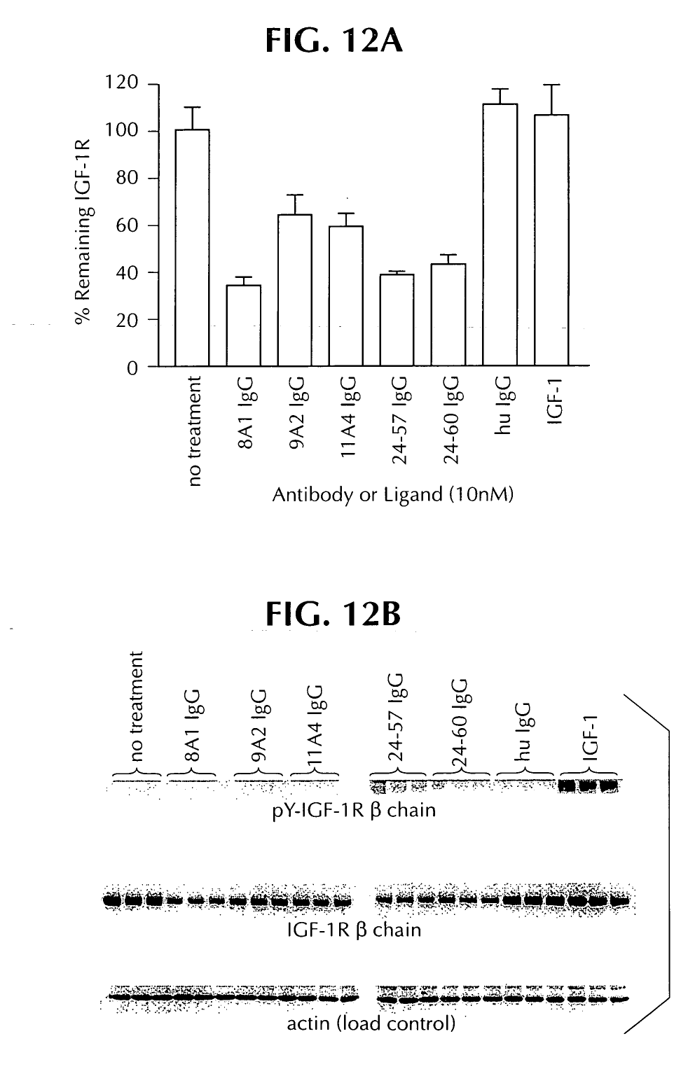

We tested the effect of Mab on IGF-1R down-regulation of IGF-1R-transfected NIH-3T3 cells by 1) measuring surface receptor levels using flow cytometry and 2) measuring total receptor levels using Western blot analysis. The experiment was performed with antibodies of the invention, particularly 8A1, 9A2, 11A4. We observed down-regulation of the IGF-1R in these cells. See, e.g., FIGS. 11 and 12. IGF-1R levels were reduced greater than 50% three hours after the addition of an antibody of the invention.

For the preparation of cells for FACS analysis, we plated IGF-1R-transfected NIH-3T3 cells in 4 ml of growth media (DMEM high glucose media supplemented with 10% heat-inactivated FBS, 0.29mg/ml L-glutamine, 1000 ug/ml penicillin and streptomycin) per well in 6-well plates. We incubated the plates at 37° C., 5% CO2 overnight to allow cells to attach. One hour before testing, we removed the media from the plates; added 4 ml of serum-free media; removed the serum free media by vacuum suction with pipettes; and added another 4 ml of serum-free media per well. For testing, we diluted the IGF-1R antibodies in serum-free media to 1 ug/ml final concentration and replaced the serum-free media in plates with 4 ml of media with or without antibodies per well at the desired time points. We then incubated the plates at 37° C. for the remaining time. At the time of harvesting the cells, we removed the culture media, washed the plates one time with cold PBS-without Ca/Mg and then replaced with 2 ml of 0.25% trypsin/EDTA (0.25% trypsin—1 mMEDTA) per well at 37° C. for 3 minutes. We then collected the trypsinized cell samples into tubes containing 5 ml of complete growth media on ice. The tubes were centrifuged at 1500 rpm for 5 minutes and the cell pellets were then washed with FACS buffer (0.1% BSA and 0.1% sodium azide in Ca and Mg-free PBS) one time. The cell number was determined. We plated 0.5−2×10⁵ cells/well in 96 well round-bottomed plates. The plates were centrifuged and we decanted the FACS buffer from the plates and replaced it with 50ul of FACS buffer containing the IgG control antibodies or the anti-IGF-1R antibodies at 10 ug/ml final concentration as the primary antibodies. We incubated the plates at 4° C. for 30 minutes. We then washed the plates two times with FACS buffer. Cells were washed by decanting the buffer via inverting the plates and blotting the plates gently onto paper towels and then replacing with new buffer for cell suspension and then the cell pellet was collected. The cells were then incubated with FITC-conjugated donkey anti-mouse or donkey anti-human antibodies diluted in FACS buffer to a concentration of 10 ug/ml for 30 minutes at 4° C. The stained cells were washed two times with FACS buffer; resuspended in 200 ul of FACS buffer; and immediately ran on a FACSCalibur Flow Cytometer (Bectin, Dickinson and Company, San Jose, Calif.) and analyzed using FlowJo software (Tree Star, Inc, San Carlos, Calif.). Fluorescence intensity was analyzed only on live cells, which were identified by light scatter. The geometric means of fluorescence intensity (mean channel fluorescence or MCF) were calculated and used to determine relative expression of IGF-1R on the cell surface.

In addition to evaluating the effect of antibodies of the invention on IGF-IR levels on transfected cells, we wished to test the ability of these antibodies to down-regulate IGF-1R from tumor cell lines. We plated A549 cells (non small cell lung cancer human line, ATCC) in 6 well clusters with DMEM/Hams F12 media (1:1) containing 2 mM L-glutamine, penicillin-streptomycin, and 10% fetal bovine serum. After reaching 90% confluence, the culture media waas replaced with 2 ml per well of fresh media containing 10 nM of the test antibodies or IGF-1. At selected times following addition of antibodies or ligand the cell monolayers were rinsed with ice-cold PBS, then scrape-harvested in 0.3 ml per well of 1% Nonidet P40, 25 mM Tris-HCl, pH 7.5, containing 0.15 M NaCl, 10% glycerol, 5 mM EDTA, and protease and phosphatase inhibitor cocktails. Following clarification by centrifuging at 10,000×g/20 min, equivalent amounts of protein from the supernatant fraction were analyzed by SDS-PAGE and Western blotting for total IGF-1R using sc-713 (Santa Cruz Biotechnology) and for actin (Sigma A-2066) for total protein loading. As shown in FIG. 13, a time dependent preferential loss of total IGF-1R was observed when A549 tumor cells were treated with 8A1, 9A2, and 11A4 IGF-1R antibodies vs. control human IgG or IGF-1. In this regard, the results obtained agreed well with those observed using NIH-3T3 fibroblasts over-expressing the human IGF-1R. Thus, we were able to demonstrate down-regulation of total IGF-1R from both fibroblasts over-expressing the human IGF-1R, as well as human tumor cell lines that express endogenous IGF-1R.

### Example 13

### IGF-1R Down-Regulation by Monoclonal Antibodies evaluated by FACS

We tested the ability of monoclonal antibodies to decrease the level of cell surface IGF-1R using NIH-3T3 fibroblasts transfected with the human IGF-1R. These experiments were performed with antibodies of the invention, particularly 8A1, 9A2, 11A4, and a commercially available mouse IGF-1R monoclonal antibody (alpha-IR3). Cells were grown in 6-well clusters to approximately 80% confluence in DMEM containing 10% fetal bovine serum. One hour before experiments were initiated the culture media was replaced with DMEM without serum (binding media), and the cells were incubated in binding media containing I ug/ml of test antibodies for up to 8 hours at 37° C./5%CO₂.

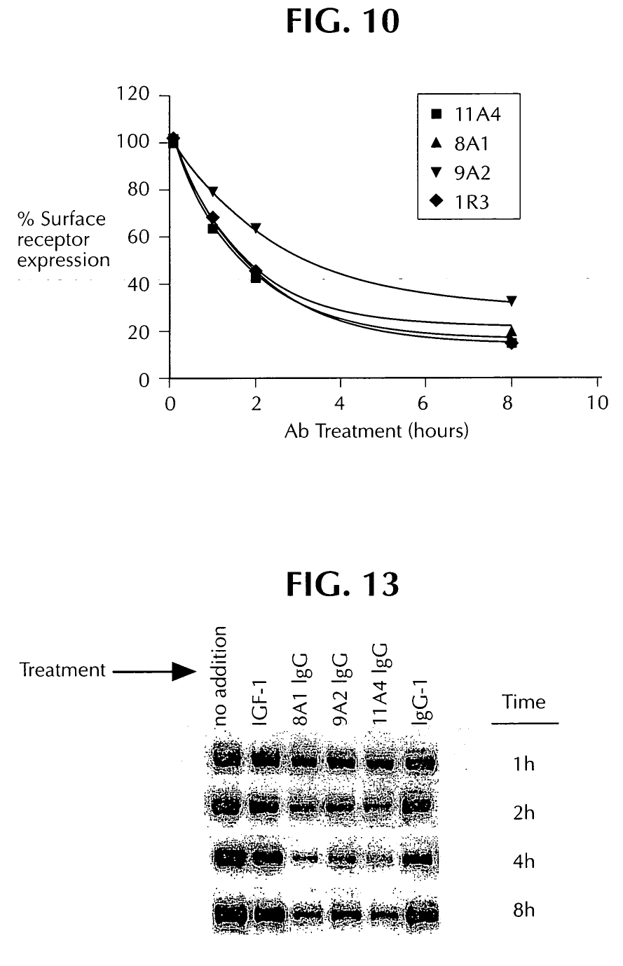

The extent of down-regulation of IGF-1R by the test monoclonal antibodies was determined by FACS analysis. At the selected time points, cells were washed once with PBS lacking Ca⁺⁺/Mg⁺⁺ and then removed from the dishes with 0.25% trypsin/EDTA. Cells from each well were collected into 5 ml of DMEM containing 10% fetal bovine serum, and collected by centrifuging at 1500 rpm for 5 min. The cell pellet was resuspended in FACS buffer (PBS lacking Ca⁺⁺/Mg⁺⁺ and containing 0.1% BSA and 0.1% sodium azide). Cells (0.5−2.0×E5) were plated into 96-well round bottom plates, centrifuged to pellet the cells as before, and resuspended in 50 ul FACS buffer containing either control IgG or their cognate IGF-1R antibody at 10 ug/ml. After 30 minutes on ice, the cells were pelleted again and washed twice with FACS buffer. Cells were then incubated for 30 minutes on ice with 10 ug/ml FITC-conjugated donkey anti-mouse IgG or donkey anti-human IgG diluted in FACS buffer. Stained cells were washed twice in FACS buffer, resuspended in 200 ul final volume of FACS buffer, and analyzed on a FACSCalibur Flow Cytometer (Becton Dickinson, San Jose, Calif.) with FlowJo software (Tree Star Inc., San Carlos, Calif.). Fluorescence intensity was analyzed only on live cells, which were identified by light scatter. The mean channel fluorescence (MCF) was calculated and used to determine relative expression of IGF-1R on the cell surface as a function of time at 37° C. The results presented in FIG. 10 indicate that all tested antibodies of the invention, were effective at decreasing the level of cell-surface IGF-1R.

### Example 14

### Epitope Mapping Studies

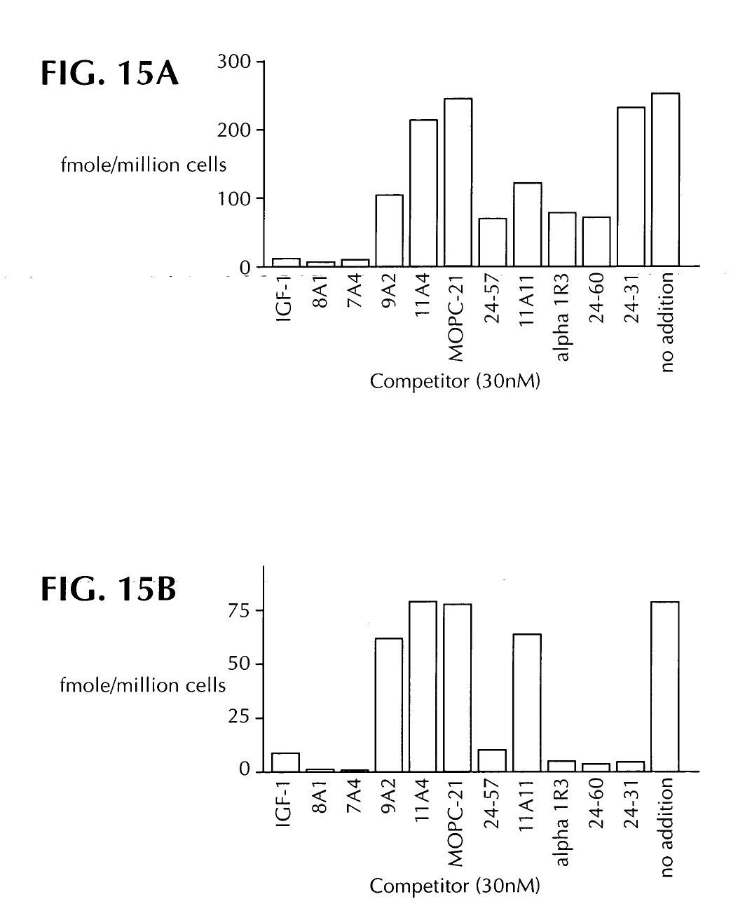

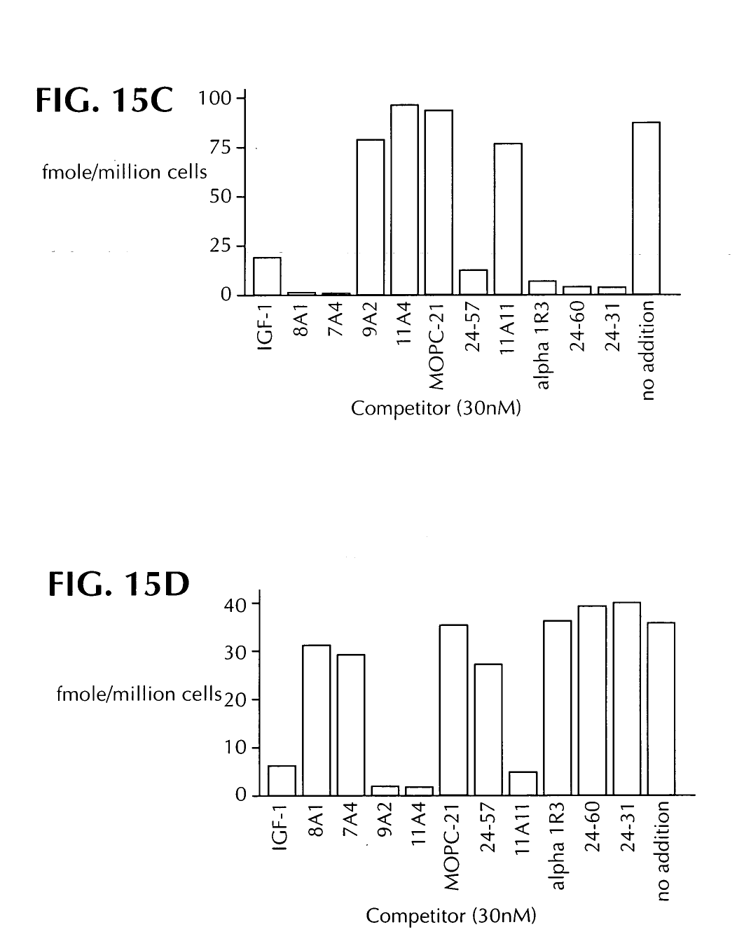

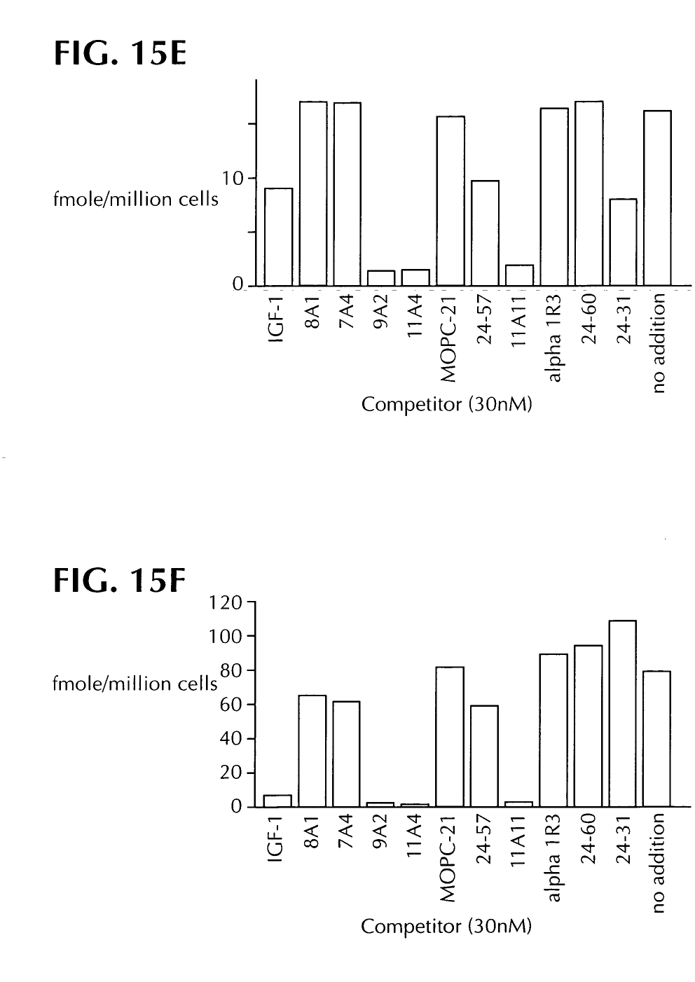

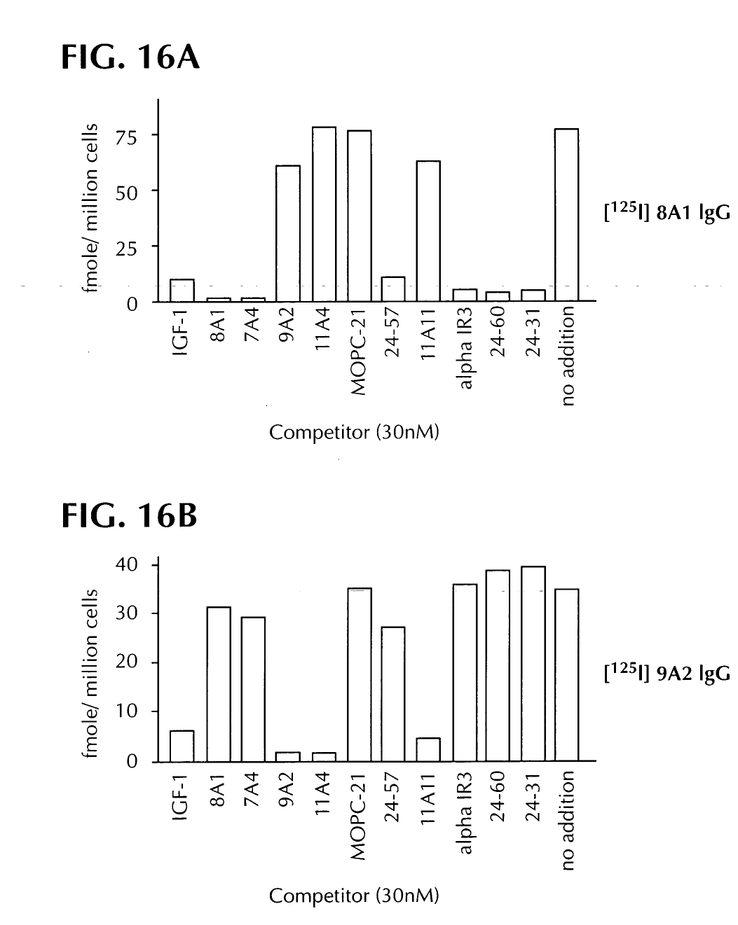

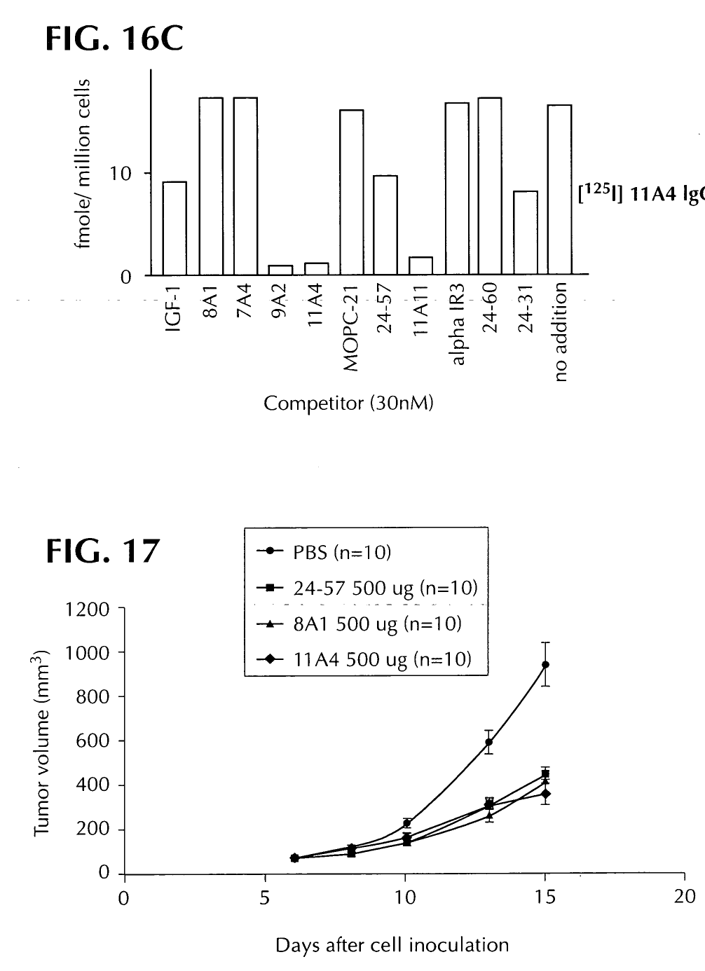

Having demonstrated that the antibodies of the invention recognize IGF-1R and block ligand binding to the IGF-1R, we performed epitope mapping studies with a subset of the antibodies of the invention. We focused these experiments particularly on the 7A4, 8A1, 9A2, 11A4, and 11A11 antibodies. We conducted competition binding assays on NIH-3T3fibroblasts expressing the human IGF-1R to evaluate whether the antibodies of the invention bind to the same or distinct sites on the IGF-1R, and compared their recognized epitopes with those already mapped onto the IGF-1R using commercially-available mouse IGF-1R monoclonal antibodies. For this purpose, we radioiodinated antibodies of the invention to a specific activity of 17.4-20.3 uCi/ug using Iodogen and standard techniques known to one skilled in the art. Radioiodinated IGF-I was purchased from a commercial source (Perkin-Elmer; #NEX241). NIH-3T3 cells stably expressing the human IGF-IR were plated at 2×E4 cells/well in 24-well tissue culture dishes in 1 ml/well of DMEM (Gibco, #11995-040, Grand Island, N.Y.) supplemented with 2 mM L-glutamine (Gibco, #25030-081) and 10% fetal bovine serum (Hyclone, #SH30070.03, Logan Utah). Cells were incubated for two days at 37° C./5% CO2 until approximately 80% confluent, and then the growth media was replaced with 1.0 ml/well of DMEM containing 20 mM Hepes (Gibco, #15630-080) and 0.5% BSA (Equitech Bio, 30% solution, protease-free, Kerrville, Tex.), and incubation continued overnight at the above temperature in this starvation media. To initiate the binding assay, dishes were placed on ice-water and the culture media was replaced with 0.25 ml/well of ice-cold starvation media containing 60 nM of the selected competitor, followed immediately by addition of an equal volume of ice-cold starvation media containing 0.6 nM of each test radiolabeled monoclonal antibodies or IGF-I. Binding was allowed to proceed for three hours at 4° C., then the cell monolayers were washed three times with 0.75 ml/well ice-cold Dulbecco's PBS (Gibco, #14070-117). Cells and associated radioactivity were released from the dishes with 0.75 ml of 2% sodium dodecyl sulfate (Gibco, #24730-020) supplemented with 0.2N NaOH and heating the dishes at 50° C. for 15 min. Lysate radioactivity was then quantitated by gamma scintillation spectrometry. Each well contained on average 1.8×E5 cells, and lysate counts per minute (CPM) were transformed to femtomoles of radioligand bound per million cells based upon the known specific activity of the radioligand. The results shown in FIG. 15 indicate that 8A1 and 7A4 antibodies of the invention are more effective competitors for IGF-I binding than the other antibodies tested under these assay conditions. In addition, 8A1 and 7A4 appear to share a common, possibly identical, IGF-1R epitope that overlaps the reported (Adams et al., Cell. Mol. Life Sci. 57:1050-1093, 2000) epitopes recognized by all tested commercial mouse anti-IGF-1R monoclonal antibodies (24-57, #MS-643-PABX, NeoMarkers, Fremont, Calif.; alpha IR3, #GR11SP2, Oncogene Research Products, San Diego, Calif.; 24-31, #MS-641-PABX, NeoMarkers; 24-60, #MS-644-PABX, NeoMarkers). In contrast, 9A2, 11A4, and 11A11 human IGF-1R appeared to recognize a distinct, but possibly shared or overlapping, IGF-1R epitope from that recognized by 7A4 and 8A1. These experiments allowed us to assign the antibodies of the invention to different binding groups. They also indicated that several antibodies of the invention appear to recognize identical or similar epitopes as commercially available mouse antibodies to the human IGF-1R. FIG. 16 indicates that there are distinct epitopes for anti-IGF-1R antibodies 8A1, 9A2, and 11A4.

### Example 15

### Inhibition of Tumor Growth/IGF-1R Expression with IGF-1R Antibodies

Establishment of Model:

3T3/IGF-1R-S cell line was used in this experiment. 1×10\hat{ }6 cells/mouse were inoculated into female nude mice sc. by 10 μl of 60% PBS/Matrigel solution. 6 days after cell injection, 70 mice (with tumors of 60-70 mm³ bearing) were randomly divided into seven groups (10 mice/group) as below. The compounds were administrated on day 7, day 10 and day 13.

Group 1, PBS, 200 μl, IP

Group 2, human IgG, 500 μg, IP

Group 3, 24-57, 500 μg, IP

Group 4, 8A1, 100 μg, IP

Group 5, 8A1, 500 μg, IP

Group 6, 11A4, 100 μg, IP

Group 7, 11A4, 500 μg, IP

Monitor:

The tumor size was recorded twice a week by venier calipers. The volume was calculated by the formula: mm³=length×(width)²×0.52. Body weight was recorded once a week.

FIG. 17 shows the results where 1×10⁶ of 3T3/IGF-1R-S cells/mouse were inoculated into female nude mice sc. by 10 ml of 60% Matrigel/PBS solution. The tumor bearing mice were randomly divided and the compounds were administrated on day 7, day 10 and day 13. All the mice were terminated on day 16. The tumor size was recorded twice a week by venier calipers. The volume was calculated by the formula: mm³=length×(width)²×0.52. Body weight was recorded once a week.

Both human mAb, 8A1 and 11A4, have significant tumor delay effects. The tumor growth inhibition effects are comparable with our surrogate mouse mAb, 24-57.

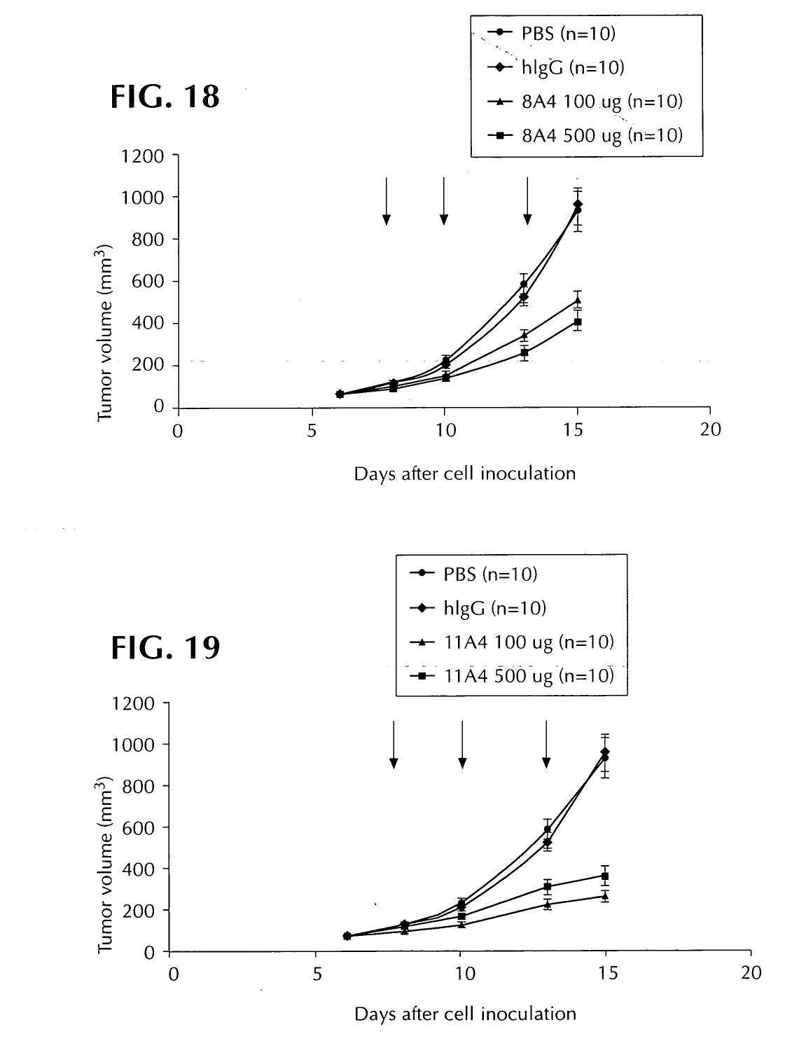

FIG. 18 shows results where 1×10\hat{ }6 of 3T3/IGF-1R-S cells/mouse were inoculated into female nude mice sc. by 10 ml of 60% Matrigel/PBS solution. The tumor bearing mice were randomly divided and the compounds were administrated on day 7, day 10 and day 13. All the mice were terminated on day 16. The tumor size was recorded twice a week by venier calipers. The volume was calculated by the formula: mm³=length×(width)²×0.52. Body weight was recorded once a week.

The amount of IGF-1R remaining at day 15 was 97.2% for the PBS control, 102.8% for the human IgG control, 18.6% for 8A1 IgG at 100 μg level, and 24.6% for 8A1 IgG at the 500 μg level. The 8A1 IgG inhibited tumor growth in vivo at either 100 μg (45% tumor delay) or 500 μg (56% tumor delay). The difference between the two treatment groups is not significant (P>0.1). These results indicate that doses above 100 μg may not be more efficacious.

FIG. 19 shows results where 1×10⁶ of 3T3/IGF-1R-S cells/mouse were inoculated into female nude mice sc. by 10 ml of 60% Matrigel/PBS solution. The tumor bearing mice were randomly divided and the compounds were administrated on day 7, day 10 and day 13. All the mice were terminated on day 16. The tumor size was recorded twice a week by venier calipers. The volume was calculated by the formula: mm³=length×(width)²×0.52. Body weight was recorded once a week.

The amount of IGF-1R remaining at day 15 was 97.3% for the PBS control, 102.7% for the human IgG control, 15.1% for 11A4 IgG at the 100 μg level, and 11.9% for 11A4 IgG at the 500 μg level. This chart showed that the dose response of 11A4. Again, we did not find any additional efficacy with a dose beyond 100 μg.

What is claimed is:

1. An antibody or antigen binding portion thereof that specifically binds to IGF-IR wherein said antibody comprises a IGF-1R antibody selected from the group consisting of PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5 or fragment of any one thereof.

2. The antibody or antigen binding portion thereof of claim wherein said IGF-1R antibody is selected from the group consisting of PINT-7A4, PINT-8A1, PINT-9A2, PINT-11A1, and PINT-11A4 or a fragment of any one thereof.

3. The antibody or antigen binding portion thereof of claim 1 wherein said IGF-1R antibody is selected from the group consisting of PINT-8A1, PINT-9A2, and PINT-11A4 or a fragment of any one thereof.

4. The antibody or antigen binding portion thereof of claim 1, 2 or 3 wherein said antibody comprises at least one light chain of said IGF-1R antibody and/or at least one heavy chain of said IGF-1R antibody.

5. The antibody or antigen binding portion thereof of claim 4 wherein said antibody comprises at least one CDR of said IGF-1R antibody.

6. The antibody or antigen binding portion thereof of claim 5, wherein said antibody comprises CDRs from different light chains of said IGF-1R antibody and/or CDRs from different heavy chains of said IGF-1R antibody.

7. The antibody or antigen binding portion thereof of claim 1, 2 or 3 wherein said antibody comprises at least one V_{L} or V_{H} variable region of said IGF-1R antibody.

8. The antibody or antigen-binding portion thereof according to claims 1, 2 or 3, wherein the antibody or portion thereof has at least one property selected from the group consisting of:
a) cross-competes for binding to human IGF-1R;
b) binds to the same epitope of human IGF;
c) binds to human IGF-1R with substantially the same K_{d}; and
d) binds to human IGF-1R with substantially the same off rate.

9. The antibody or antigen-binding portion thereof according to claims 4 wherein the antibody or portion thereof has at least one property selected from the group consisting of:
a) cross-competes for binding to human IGF-1R;
b) binds to the same epitope of human IGF;
c) binds to human IGF-1R with substantially the same K_{d}; and
d) binds to human IGF-1R with substantially the same off rate.

10. The antibody or antigen-binding portion thereof according to claims 5 wherein the antibody or portion thereof has at least one property selected from the group consisting of:
a) cross-competes for binding to human IGF-1R;
b) binds to the same epitope of human IGF;
c) binds to human IGF-1R with substantially the same K_{d}; and
d) binds to human IGF-1R with substantially the same off rate.

11. The antibody or antigen-binding portion thereof according to claim 6 wherein the antibody or portion thereof has at least one property selected from the group consisting of:
a) cross-competes for binding to human IGF-1R;
b) binds to the same epitope of human IGF;
c) binds to human IGF-1R with substantially the same K_{d}; and
d) binds to human IGF-1R with substantially the same off rate.

12. The antibody or antigen-binding portion thereof according to claim 7 wherein the antibody or portion thereof has at least one property selected from the group consisting of:
a) cross-competes for binding to human IGF-1R;
b) binds to the same epitope of human IGF;
c) binds to human IGF-1R with substantially the same K_{d}; and
d) binds to human IGF-1R with substantially the same off rate.

13. The antibody or antigen-binding portion thereof according to claim 1, 2 or 3 wherein said antibody is an immunoglobulin G (IgG), an IgM, an IgE, an IgA or an IgD molecule.

14. The antibody or antigen-binding portion thereof according to claim 1, 2 or 3 wherein said antibody is an Fab fragment, an F(ab′)2 fragment, an Fv fragment, or a single chain antibody.

15. The antibody or antigen-binding portion thereof according to claim 1, 2 or 3 wherein said antibody is a humanized antibody, a human antibody, a chimeric antibody or a bispecific antibody.

16. The antibody of claim 13 wherein said IGF-1R antibody is an IgG selected from the group consisting of PINT-7A4, PINT-8A1, PINT-9A2, PINT-11A1, and PINT-11A4.

17. A pharmaceutical composition comprising the antibody or portion thereof according to claims 1, 2 or 3 and a pharmaceutically acceptable carrier.

18. An isolated cell line that produces the antibody according to claims 1, 2 or 3

19. A method or treatment of a cancer or tumor comprising administering an effective amount of an IGF-1R specific antibody of claims 1, 2 or 3.

20. An isolated nucleic acid molecule that comprises a nucleic acid sequence that encodes a heavy chain or antigen-binding portion thereof or a light chain or antigen-binding portion thereof of an antibody according to claims 1, 2 or 3.
